SEQUENCE LISTING (13 sequences)

SEQ ID NO: 1 - protein, 156 aa, Mus musculus
  REGION 1..156: note = MISC_FEATURE - murine C1 domain
CSTQLGMEGGAIADSQISASSVYMGFMGLQRWGPELARLYRTGIVNAWTASNYDSKPWIQVNLLRKMRVSGVMTQGASRAGRAEYLKTFKVAYSLDGRKFEFIQDESGGDKEFLGNLDNNSLKVNMFNPTLEAQYIKLYPVSCHRGCTLRFELLGC

SEQ ID NO: 2 - protein, 156 aa, Mus musculus
  REGION 1..156: note = MISC_FEATURE - murine C2 domain
CSEPLGLKNNTIPDSQMSASSSYKTWNLRAFGWYPHLGRLDNQGKINAWTAQSNSAKEWLQVDLGTQRQVTGIITQGARDFGHIQYVASYKVAHSDDGVQWTVYEEQGSSKVFQGNLDNNSHKKNIFEKPFMARYVRVLPVSWHNRITLRLELLGC

SEQ ID NO: 3 - protein, 387 aa, Homo sapiens
  REGION 1..387: note = MISC_FEATURE - full length human MFG-E8
MPRPRLLAALCGALLCAPSLLVALDICSKNPCHNGGLCEEISQEVRGDVFPSYTCTCLKGYAGNHCETKCVEPLGLENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 4 - protein, 463 aa, Mus musculus
  REGION 1..463: note = MISC_FEATURE - full length mouse MFG-E8 protein
MQVSRVLAALCGMLLCASGLFAASGDFCDSSLCLNGGTCLTGQDNDIYCLCPEGFTGLVCNETERGPCSPNPCYNDAKCLVTLDTQRGDIFTEYICQCPVGYSGIHCETETNYYNLDGEYMFTTAVPNTAVPTPAPTPDLSNNLASRCSTQLGMEGGAIADSQISASSVYMGFMGLQRWGPELARLYRTGIVNAWTASNYDSKPWIQVNLLRKMRVSGVMTQGASRAGRAEYLKTFKVAYSLDGRKFEFIQDESGGDKEFLGNLDNNSLKVNMFNPTLEAQYIKLYPVSCHRGCTLRFELLGCELHGCSEPLGLKNNTIPDSQMSASSSYKTWNLRAFGWYPHLGRLDNQGKINAWTAQSNSAKEWLQVDLGTQRQVTGIITQGARDFGHIQYVASYKVAHSDDGVQWTVYEEQGSSKVFQGNLDNNSHKKNIFEKPFMARYVRVLPVSWHNRITLRLELLGC

SEQ ID NO: 5 - protein, 316 aa, Mus musculus
  REGION 1..316: note = MISC_FEATURE - mouse PS-binding domain (C1+C2 domain)
CSTQLGMEGGAIADSQISASSVYMGFMGLQRWGPELARLYRTGIVNAWTASNYDSKPWIQVNLLRKMRVSGVMTQGASRAGRAEYLKTFKVAYSLDGRKFEFIQDESGGDKEFLGNLDNNSLKVNMFNPTLEAQYIKLYPVSCHRGCTLRFELLGCELHGCSEPLGLKNNTIPDSQMSASSSYKTWNLRAFGWYPHLGRLDNQGKINAWTAQSNSAKEWLQVDLGTQRQVTGIITQGARDFGHIQYVASYKVAHSDDGVQWTVYEEQGSSKVFQGNLDNNSHKKNIFEKPFMARYVRVLPVSWHNRITLRLELLGC

SEQ ID NO: 6 - protein, 12 aa, synthetic construct
  REGION 1..12: note = linker
GSAGSAAGSGEF

SEQ ID NO: 7 - protein, 5 aa, synthetic construct
  REGION 1..5: note = sortase A recognition sequence
  VARIANT 3: note = Xaa is any amino acid
LPXTG

SEQ ID NO: 8 - protein, 5 aa, synthetic construct
  REGION 1..5: note = sortase A recognition sequence
  VARIANT 3: note = Xaa is any amino acid
LPXTA

SEQ ID NO: 9 - protein, 8 aa, synthetic construct
  REGION 1..8: note = sortase B recognition sequence
  VARIANT 2: note = Xaa is any amino acid
DXPNTGDE

SEQ ID NO: 10 - DNA, 28 bp, synthetic construct
  misc_feature 1..28: note = Priming sequence at 5' end
  source 1..28: mol_type = other DNA
tgtttaactttaagaaggagatatacat

SEQ ID NO: 11 - DNA, 31 bp, synthetic construct
  misc_feature 1..31: note = Priming sequence at 3' end
  source 1..31: mol_type = other DNA
taactcgagcaccaccaccaccaccactgag

SEQ ID NO: 12 - protein, 11 aa, synthetic construct
  REGION 1..11: note = Sequence at 3'-end of C1 and C2 fragments in cloning
GSLPETGGSLE

SEQ ID NO: 13 - protein, 5 aa, synthetic construct
  REGION 1..5: note = Sortase recognition sequence
LPETG

(19) United States
(12) Patent Application Publication
(10) Pub. No.: US 2024/0125806 A1
(43) Pub. Date: Apr. 18, 2024

(54) PHOSPHATIDYLSERINE BINDING AGENTS FOR THE DETECTION AND DEPLETION OF PHOSPHATIDYLSERINE POSITIVE CELLS

(71) Applicants: BIOLEGEND, INC., San Diego, CA (US); TBJK GmbH, Herrsching (DE)

(72) Inventors: THOMAS BROCKER, Herrsching (DE); JAN KRANICH, Muenchen (DE); TILMAN KURZ, Muenchen (DE); HONG ZHANG, San Diego, CA (US); HYUN PARK, San Diego, CA (US)

(73) Assignees: BIOLEGEND, INC., San Diego, CA (US); TBJK GmbH, Herrsching (DE)

(21) Appl. No.: 18/377,228

(22) Filed: Oct. 5, 2023

Related U.S. Application Data
(63) Continuation of application No. PCT/US2022/024906, filed on Apr. 14, 2022.
(60) Provisional application No. 63/178,427, filed on Apr. 22, 2021.

(51) Int. Cl. G01N 33/92; C07K 14/485; C12N 9/52; G01N 33/569

(52) U.S. Cl. CPC G01N 33/92; C07K 14/485; C12N 9/52; C12Y 304/2207; G01N 33/56966; C07K 2319/60; G01N 2405/04

(57) ABSTRACT

The present invention relates to a phosphatidylserine (PS) binding agents comprising one or more C-domains of a milk fat globule-EGF factor 8 (MFG-E8) protein, and methods of making and using the same.

### CROSS-REFERENCE TO RELATED APPLICATION

This application is a continuation of PCT/US2022/024906, filed Apr. 14, 2022, which application claims priority to U.S. Provisional Application No. 63/178,427, filed Apr. 22, 2021, entitled “PHOSPHATIDYLSERINE BINDING AGENTS FOR THE DETECTION AND DEPLETION OF PHOSPHATIDYLSERINE POSITIVE CELLS”, the contents of which are hereby incorporated by reference in their entirety for all purposes.

### INCORPORATION BY REFERENCE OF SEQUENCE LISTING

The present application contains a Sequence Listing which has been submitted electronically in XML file format and is hereby incorporated by reference in its entirety. Said XML copy, created on Nov. 3, 2023, is named 102738-1410360-002011US_SL.xml and is 20,035 bytes in size.

### TECHNICAL FIELD OF THE DISCLOSURE

The present disclosure relates to a phosphatidylserine (PS) binding agent comprising one or more (e.g., at least two) isolated C-domains of a milk fat globule-EGF factor 8 (MFG-E8) protein. Further, the present disclosure relates to a composition comprising the at least one PS binding agent, a kit comprising the PS binding agent or the composition and use of the PS binding agent, the composition or the kit for staining or depleting PS-positive dead or dying cells, cell debris, activated thrombocytes and/or extracellular vesicles. Also the present disclosure relates to a kit comprising two or more isolated C-domains of a MFG-E8 protein and an additional molecule capable of linking said C-domains to each other. The present disclosure also relates to an in vitro method of staining or depleting PS-positive dead or dying cells, cell debris, activated thrombocytes and/or extracellular vesicles, an in vitro method of diagnosing a diseases associated with the presence of PS positive dead or dying cells, cell debris, activated thrombocytes and/or extracellular, a method of producing the PS binding agent of the present disclosure, and an in vitro method of detecting PS-positive dead or dying cells, cell debris, activated thrombocytes and/or extracellular vesicles in an animal to whom a PS binding agent has been pre-delivered.

### BACKGROUND

The clearance of apoptotic and/or necrotic cells is crucial for the prevention of autoimmune and inflammatory responses against self-antigens released by dead or dying cells. Apoptotic and/or necrotic cells display phosphatidylserine (PS) on their outer cell surface, where it acts as an ‘eat-me’ signal and contributes to efficient removal of dead cells by macrophages and other phagocytes (Fadok, Voelker et al. 1992, J Immunol 148(7): 2207-2216). Lactadherin or milk fat globule-EGF factor 8 protein (MFG-E8) functions as an important mediator of dead cell removal, as it facilitates engulfment of apoptotic and/or necrotic cells by phagocytes. MFG-E8 is a large glycosylated soluble protein produced by various cell types and tissues. At its N-terminal side, the murine homologue carries two EGF-like domains, followed by two C-domains, C1 and C2. In addition, it has a proline-threonine rich domain which, however, splice variants exist lacking this domain (Hanayama, Tanaka et al. 2002, Nature 417(6885): 182-187). With an RGD motif in the second EGF-like domain, MFG-E8 can bind to integrin family receptors such as αᵥβ₃ and αᵥβ₅ on phagocytes. MFG-E8 binds to PS on apoptotic and/or necrotic cells (Peterson, Patton et al. 1998, Biol Neonate 74(2): 143-162) mainly via its C2 domain (Otzen, Blans et al. 2012, Biochim Biophys Acta 1818(4): 1019-1027, Ye, Li et al. 2013, Biochim Biophys Acta 1828(3): 1083-1093). Thereby, MFG-E8 links phagocytes and dead cells for efficient removal of the latter.

Isolated MFG-E8 C-domains have been expressed recombinantly and the C2-domain was used as a biosensor to detect intracellular PS-distribution and intracellular vesicles (Yeung, Gilbert et al. 2008). The binding of MFG-E8 to PS' membranes was characterized to be a 2-step process entirely mediated by the C2-domain (Otzen, Blans et al. 2012, Biochim Biophys Acta 1818(4): 1019-1027). Accordingly, NMR and structural analyses of bovine and murine MFG-E8 C2-domains revealed that binding of C2 to PS is selective (Shao, Novakovic et al. 2008, J Biol Chem 283(11): 7230-7241) and mediated by positive charges of C2 as well as hydrophobic interactions via solvent-exposed aromatic amino acid residues (Ye, Li et al. 2013). Full length MFG-E8 as well as its single C-domains were difficult to manufacture in large scale, as both, C1- and C2-domains are responsible for protein aggregation, when expressed by insect or eukaryotic cells. Therefore, for reasons of feasibility previous studies rather generated C2-domain derived cyclic peptides mimicking the C2-PS-binding portion.

However, some studies also revealed that the binding to PS⁺-membranes by isolated C2-domains would be less efficient as compared to other PS⁺-binding molecules (Ye, Li et al. 2013) and approx. 100-fold reduced than by full length MFG-E8 (Shao, Novakovic et al. 2008).

Thus, within the MFG-E8 molecule, the PS binding activity has not been shown for its C1-domain so far, only for its C2-domain, both of which have been described to be difficult to manufacture in large scale with site-specific tags.

In addition to the above mentioned, the prior art mainly uses Annexin V, which is one of the most widely used reagents used to stain apoptotic and/or necrotic cells in flow cytometric analyses. However, binding of Annexin V to PS is dependent on high concentrations of Ca²⁺, which often makes meaningful work more difficult. Thus, when using Annexin V the cells have to be stained in a special, Ca²⁺-containing binding buffer (van Engeland, M et al. 1998, Cytometry 31, 1-9).

Therefore, there is a need in the art to provide new PS binding agents for the detection and removal of dead or dying cells which are effective and preferably at the same time buffer-independent in comparison to the agents used in the prior art so far.

Therefore, the objective of the present invention is to comply with this need.

The solution of the present invention is described in the following, exemplified in the appended examples, illustrated in the figures and reflected in the claims.

### BRIEF SUMMARY

The present invention relates to the binding of isolated MFG-E8 C1- and C2-domains to phosphatidlyserine (PS) positive (PS⁺) dead or dying cells, cell debris, activated thrombocytes and/or extracellular vesicles. The inventors successfully expressed N-terminally tagged single C1- and C2-domains in bacteria, isolated large amounts of recombinant protein and stabilized them with specific buffer systems (see FIGS. 1A-1D). It was shown that monomeric C1- and C2-domains of murine MFG-E8 protein have almost no detectable PS-binding capacities. However, when the inventors multimerized C1- and/or C2-domains having a site-specific modification with a biotin-containing peptide with homotetrameric streptavidin (SA) to a multimeric assembly comprising at least two C-domains (see FIGS. 2A and 2B), C1- and C2-multimers both showed a strong binding to PS⁺ dead or dying cells, cell debris, activated thrombocytes and/or extracellular vesicles (see FIGS. 3A-3C and FIG. 9). It was further demonstrated by the inventors of the present invention that C1-multimers under physiological conditions bind dead cells more potently when compared to C2-multimers (see FIGS. 4A and 4B), wherein C1-multimers detect approximately the same frequency of PS positive cells as compared to Annexin V (see FIGS. 5A and 5B). Hence, C1-multimers seem to be an effective and buffer-independent alternative to known PS binding molecules and can thus be employed as Ca′-independent detection reagent of PS positive dead or dying cells, cell debris, activated thrombocytes and/or extracellular vesicles. Hence, the PS binding agents of the present invention can be applied for staining PS⁺ dead or dying cells, cell debris, activated thrombocytes and/or extracellular vesicles. Furthermore, the inventors showed that C1-multimers can be used for highly efficient removal of necrotic and apoptotic cells or debris from cell suspension (see FIGS. 6A-6D, 10A, 10B, 11A, and 11B), as well as detection of PS⁺ membranes such as PS positive extracellular vesicles (EVs) a sample obtained from a subject (see FIGS. 7A, 7B, 8A, and 8B). Hence, the present invention proves that C1-multimers are well suitable to analyze cell death and EVs associated to living cells.

In sum, the present invention provides a distinct and versatile recombinant molecule specific for PS positive dead or dying cells, cell debris, activated thrombocytes and/or EVs with multiple applications in vitro with tremendous potential in diagnosis and imaging, assays.

Accordingly, in a first aspect the present invention relates to a phosphatidlyserin (PS) binding agent comprising one or more (e.g., at least two)C-domains of a milk fat globule-EGF factor 8 (MFG-E8) protein. In some embodiments, provided herein is a phosphatidlyserin (PS) binding agent comprising one or more (e.g., at least two) isolated C-domains of a milk fat globule-EGF factor 8 (MFG-E8) protein. In some of any embodiments, the C-domains of the PS binding agent are isolated. In some of any embodiments, the C-domains of the PS binding agent do not comprise EGF or EGF-like domains.

In another aspect, the present invention refers to a composition comprising at least one PS binding agent defined elsewhere herein. In some embodiments, the PS binding agent comprises two, three, four, or more C-domains of the MFG-E8 protein.

Also provided herein is a PS binding agent comprising a backbone and one or more isolated C domains of a MFG-E8 protein. In some of any embodiments, the backbone is an additional molecule capable of linking said C-domains to each other. In some of any embodiments, the backbone comprises streptavidin, avidin, or neutravidin.

In some embodiments of this aspect, the C-domains comprise a C1-domain of the MFG-E8 protein or a functional fragment thereof, and a C2-domain of the MFG-E8 protein or a functional fragment thereof. In other embodiments, the PS binding agent comprises one or more C-domains, which can be a C1-domain of the MFG-E8 protein or a functional fragment thereof, or a C2-domain of the MFG-E8 protein or a functional fragment thereof.

In some embodiments, the C1-domain comprises an amino acid sequence having at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of SEQ ID NO: 1. In certain embodiments, the C1-domain comprises the amino acid of SEQ ID NO: 1. In some embodiments, the C2-domain comprises an amino acid sequence having at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of SEQ ID NO: 2. In certain embodiments, the C2-domain comprises the amino acid sequence of SEQ ID NO: 2.

In some embodiments, the PS binding agent comprises two or more C-domains and the C-domains are covalently linked.

In some embodiments, the PS binding agent further comprises a recognition sequence for enzymatic modification. In certain embodiments, the recognition sequence is for a biotin ligase or for a sortase A. In certain embodiments, the recognition sequence for the sortase A comprises the amino acid sequence of SEQ ID NO: 7 or SEQ ID NO: 8.

In some embodiments, the one or more C-domains are covalently attached to a biotin. In some embodiments, the one or more C-domains are covalently attached to the biotin by a sortase A or a biotin ligase.

In some embodiments, the PS binding agent comprises two or more C-domains that are linked directly to each other.

In some embodiments, the PS binding agent comprises two or more C-domains that are linked to each other via an additional molecule. In some embodiments, the additional molecule comprises a backbone. In some embodiments, the backbone or additional molecule can be streptavidin, avidin, or neutravidin.

In some embodiments, the PS binding agent further comprises a label. In some embodiments, the label is selected from the group consisting of a tag, a fluorophore, a solid phase carrier, a microbubble, a linker, and an active site probe. In some embodiments, the label is a solid phase carrier that comprises a bead.

In some embodiments, the PS binding agent is capable of binding to PS on dead cells, dying cells, cell debris, activated thrombocytes, and/or extracellular vesicles.

In another aspect, the present invention is also directed to a composition comprising one or more PS binding agents described herein.

In still another aspect, the present invention refers to a kit comprising the PS binding agent or the composition as defined herein.

In a further aspect, the present invention also relates to a kit comprising two or more C-domains of a MFG-E8 protein, and an additional molecule capable of linking said C-domains to each other. In some embodiments, the additional molecule comprises a backbone. In some embodiments, the additional molecule comprises streptavidin, avidin, or neutravidin.

In some embodiments of the kits described herein, the one or more C-domains comprise 1, 5, 10, 20, 30, 40, 50, 60, 70, 80, 90, or 100 or more C-domains. In certain embodiments, the one or more C-domains comprise a recognition sequence for enzymatic modification. In some embodiments of the kits described herein, the kits further comprise an enzyme for enzymatic modification of the C-domains. In certain embodiments, the C-domains are covalently attached to a biotin. In some embodiments of the kits described herein, the kits further comprise a buffer that is substantially free of calcium.

In another aspect, the present invention relates to the use of the PS binding agent as defined elsewhere herein, the composition as defined elsewhere herein, or the kit as defined elsewhere herein for staining PS-positive dead or dying cells, cell debris, activated thrombocytes and/or extracellular vesicles or for depleting PS-positive dead or dying cells, cell debris, activated thrombocytes and/or extracellular vesicles.

In still another aspect, the present invention also encompasses an in vitro method of staining PS-positive dead or dying cells, cell debris, activated thrombocytes and/or extracellular vesicles in a sample from a subject, the method comprising: a) contacting said sample with a PS binding agent as defined herein and a fluorophore, under conditions to bind the PS binding agent to the sample; and b) detecting said PS binding agent using an imaging method.

In a further aspect, the present invention also relates to an in vitro method of depleting or enriching PS-positive dead or dying cells, cell debris, activated thrombocytes and/or extracellular vesicles from or in a sample comprising living cells, the method comprising: a) contacting a sample from a subject to be diagnosed with a PS binding agent described herein and a fluorophore, under conditions to bind the PS binding agent to the sample; b) detecting the PS binding agent using an imaging method, thereby collecting imaging data; and c) comparing the imaging data received in step b) to a reference imaging data. In some embodiments, an increased signal in the imaging data in the sample from the subject as compared to the reference imaging data indicates the presence of a disease associated with the presence of PS positive dead or dying cells, cell debris, activated thrombocytes, and/or extracellular vesicles in the subject.

In another aspect, the present invention also relates to an in vitro method of depleting or enriching PS-positive cells from a sample, comprising: a) contacting said sample with a PS binding agent as defined herein, under conditions to bind the PS binding agent to the sample; b) contacting said sample with a label capable of binding to the PS binding agent; and c) depleting or enriching PS-positive cells bound to the PS binding agent. In some embodiments, the PS-positive cells comprise dead or dying cells, cell debris, activated thrombocytes, and/or extracellular vesicles.

In some embodiments, the conditions to bind the one or more PS binding agents to the sample comprise an additional molecule selected from streptavidin, avidin, or neutravidin. In some embodiments, the additional molecule links the two or more C-domains of the one or more PS binding agents. In some embodiments, the depleting or enriching PS-positive cells comprises using a force selected from a magnetic field or buoyant/floatation properties.

In another aspect, the present invention also relates to an in vitro method of depleting or enriching PS-positive cells from a sample, comprising: a) contacting the sample with one or more PS binding agents described herein; b) contacting the sample with an additional molecule selected from streptavidin, avidin, or neutravidin, wherein the additional molecule links the two or more C-domains of the one or more PS binding agents; c) contacting the sample with a label capable of binding to the PS binding agent, wherein the label comprises a fluorophore, a solid phase carrier, or a microbubble; and d) depleting or enriching PS-positive cells using a force selected from a magnetic field or buoyant/floatation properties, wherein the PS-positive cells comprise dead or dying cells, cell debris, and/or extracellular vesicles bound to the PS binding agent. In some embodiments, the solid phase carrier comprises a magnetic bead.

In another aspect, the present invention also relates to an in vitro method of depleting or enriching PS-positive cells from a sample, comprising: a) contacting the one or more PS binding agents described herein with an additional molecule selected from streptavidin, avidin, or neutravidin, wherein the additional molecule links the two or more C-domains of the one or more PS binding agents; b) contacting the one or more PS binding agents with a label capable of binding to the PS binding agent, wherein the label comprises a fluorophore, a solid phase carrier, or a microbubble; c) contacting the one or more PS binding agents with the sample, under conditions to bind the PS binding agent to the sample; and d) depleting or enriching PS-positive cells using a force selected from a magnetic field or buoyant/floatation properties, wherein the PS-positive cells comprise dead or dying cells, cell debris, and/or extracellular vesicles bound to the PS binding agent. In some embodiments, the solid phase carrier comprises a magnetic bead.

In some embodiments of the methods, the conditions comprise a buffer that is substantially free of calcium.

Also provided is an in vitro method of depleting or enriching PS-positive dead or dying cells, cell debris, activated thrombocytes and/or extracellular vesicles from or in a sample comprising living cells, the method comprising a) contacting said sample with a PS binding agent as defined herein further comprising a label, and b) depleting or enriching PS-positive dead or dying cells, cell debris, activated thrombocytes and/or extracellular vesicles bound to said PS binding agent.

In another aspect, a method of producing the PS binding agent comprising at least two C-domains of an MFG-E8 protein, the method comprising (a) contacting at least two C-domains of the MFG-E8 protein with an additional molecule capable of linking said C-domains to each other under conditions allowing the formation of a PS binding agent comprising at least two C-domains of the MFG-E8 protein, and optionally (b) recovering the produced PS binding agent is provided by the present invention.

In another aspect, the present invention also refers to an in vitro method of diagnosing a diseases associated with the presence of PS positive dead or dying cells, cell debris, activated thrombocytes and/or extracellular, the method comprising a) contacting a sample from a subject to be diagnosed with a PS binding agent as defined herein comprising a fluorophore, b) detecting said PS binding agent using an imaging method, thereby collecting imaging data, and c) comparing the imaging data received in step b) to reference imaging data.

In another aspect, the present invention refers to an in vitro method of detecting PS-positive dead or dying cells, cell debris, activated thrombocytes and/or extracellular vesicles, preferably PS-positive cell-associated extracellular vesicles in an animal, preferably a mouse, to whom a PS binding agent as defined elsewhere herein comprising a fluorophore has been pre-delivered, comprising: (a) analyzing a sample obtained from said animal, (b) detecting said pre-delivered PS binding agent using an imaging method, thereby collecting imaging data, (c) comparing the imaging data received in step b) to reference imaging data.

### BRIEF DESCRIPTION OF THE DRAWINGS

FIGS. 1A-1D: mMFG-E8 single domains expressed in E. coli and biotinylated in a sortase-reaction. (FIG. 1A) C1 was expressed with a C-terminal LPETG (SEQ ID NO: 13) motif and His₆ tag. It was immobilized on Ni-NTA beads (‘beads aft. aff. chr.’) and the GST-SUMO3 tag cleaved off by Senp2 (‘beads aft. Senp2 cleavage’) in two consecutive rounds (1^{st} and 2^{nd} flow-through Senp2). C1 co-eluted with contaminating proteins (‘eluate (non-red.)’). (FIG. 1B) The final product, pure C1 was obtained after size exclusion chromatography. (FIG. 1C) C1 and C2 lose their C-terminal His₆ tag in the sortase A reaction upon linkage to the biotin-containing peptide, a slight down-shift of the protein bands is visible. (FIG. 1D) C1-biotin and C2-biotin were further purified via size exclusion chromatography. A side product (#) of the sortase reaction is visible in the C2 elution profile.

FIGS. 2A and 2B: Fully assembled multimers show distinct band patterns independent of the SA variant. (FIG. 2A) SA(FITC) was titrated with biotinylated monomers in increasing amounts ranging from a stoichiometric ratio of 1:1 to 1:6. Products were analyzed on SDS-PAGE to determine saturation of the SA-biotin interaction. (FIG. 2B) Multimers of C1 were generated with uncoupled SA, SA(FITC) and SA(AF647) and analyzed by SDS-PAGE.

FIGS. 3A-3C: Both C1- and C2-multimers are capable of staining dead cells. (FIG. 3A) Equal amounts of C1- and C2-biotin were analyzed by SDS-PAGE, either as monomers or pre-assembled with five-fold molar excess SA(FITC) as multimers. Samples were not boiled to preserve integrity of the multimers (left panel). Only two protein species each were used in assembly (boiled samples, right side). (FIG. 3B) Depiction of the staining workflow for cells stained with C1- and C2-monomers or multimers. (FIG. 3C) Mouse thymocytes, treated with the apoptosis inducer staurosporine (+Stauro) or left untreated (-Stauro) were stained with the viability dye eFluor780 and equivalent amounts of C1- or C2-monomers plus SA-FITC or pre-assembled C1- or C2-multimers as indicated in (FIG. 3B). Numbers in the gates indicate the percentage of cells shown in the respective gates.

FIGS. 4A and 4B: mC1 multimers and mC2 multimer binding properties. Staining of necrotic cells (FIG. 4A) and apoptotic cells (FIG. 4B) with C1- and C2-multimers was performed as indicated in FIG. 3B and gating analyses were performed as shown in FIG. 3C. Geometric mean fluorescence intensity (MFI) analysis of the cells in the respective gates as shown in (FIG. 3C) was analyzed. Statistical test was calculated with five pairs in triplicates (ratio-paired t test, two-tailed) and revealed significant differences for staining with C1- vs. C2-multimers of necrotic (p=0.0002; A) and apoptotic cells (p=0.0006; B).

FIGS. 5A and 5B: C1-multimer is capable of stainingapoptotic cells. Thymocytes were treated with staurosporine to induce apoptosis and analyzed by flow cytometry. Cells were stained with Annexin V (FITC) (FIG. 5A) or MFG-E8-GFP (FIG. 5B) and C1-multimer (AF647) either in single staining or in the combinations as indicated above each plot. Intact cells were determined first using SSC/FSC gating. Cells in the SSC/FSC gate were further analyzed either in staining with (FIG. 5A) or without (FIG. 5A, FIG. 5B) Ca²⁺ as indicated.

FIGS. 6A-6D: C1-multimers can be utilized to remove dead cells from cell suspensions. (FIG. 6A) Mouse splenocytes were prepared and stained with live/dead eFluor780 and C1-multimer (FITC). Cells were either left untreated (before depl.), added to anti-Streptavidin antibody coated beads for depletion of PS positive particles (+beads) or underwent the procedure without addition of beads (-beads). Gates show: GATE I: viability dye⁺/C1-multimer^{high′} necrotic cells and debris; GATE II: viability dye⁻/C1-multimer^{high′} apoptotic cells; GATE III: viability dye⁻/C1-multimer^{low′} cells in an early apoptotic stage; and GATE IV: viability dye⁻/C1-multimer⁻ live cells. Numbers indicate percentages in the respective gates. (FIG. 6B) Analysis of depletion by C1-multimer particles using the gates I, II and III as shown in (FIG. 6A). Four independent experiments, statistical significance p<0.05; ratio paired t test, two-tailed. (FIG. 6C) Analysis of live cells and increase of cell purity from the C1-multimer depletion approach using the gate IV as shown in (FIG. 6A). Relative depletion efficiency of necrotic and apoptotic cells was calculated by taking percent of live/dead⁺C1-multimer⁺ necrotic cells and live/dead⁻C1-multimer⁺ apoptotic cells before depletion as 100% and calculating the depleted fraction as percentage thereof. Pooled data from four independent experiments is shown (unpaired t test, two-tailed). (FIG. 6D) Analysis of total cell recovery using flow cytometry. Samples of C1-multimer-depleted cells were analyzed before depletion and after addition of beads and events counted using a constant flow rate and recording time. Prior to analysis, autofluorescent particles were gated out. (Four independent experiments; ratio paired t test, two-tailed); **: p=0.001-0.01; ***: p=0.0001-0.001; ****: p<0.0001; ‘ns’ no statistical significance).

FIGS. 7A and 7B: Identification of dying cells and EV⁺ cells with C1-multimers in vivo. (FIG. 7A) Non-infected and LCMV_{Arm} (2×10⁵ PFU, i.p.) infected mice were injected with 50 μg C1-multimers (SA-AF647) on day 5 post infection. 1 h later mice were sacrificed and spleen cells were analyzed by imaging flow cytometry. Dot plots show live (live/dead⁻) single cells. C1-multimer⁺ cells were gated using un-injected controls. TIF-images of C1-multimer+ cells were analyzed using a convolutional autoencoder (CAE) as described by (Kranich, Chlis et al. 2020) and sorted into apoptotic and EV⁺ cells. (FIG. 7B) Images show examples of C1-multimer stained CAE-sorted apoptotic (left) and EV⁺ (right) cells. Scale bars =7 μm.

FIGS. 8A and 8B: Identification of dying cells and EV⁺ cells in human blood samples. Whole EDTA-blood samples from a healthy donor and a COVID-19 patient where stained with C1-multimer (SA-FITC) to identify apoptotic and EV⁺ cells. (FIG. 8A) Left dot plots show C1-multimer staining of single cells after removal of live/dead⁺CD14⁺CD16⁺CD19⁺CD56⁺ cells. C1-multimer⁺ cells were gated using an unstained control. Right dot plots show live, EV⁺ and apoptotic CD4⁺ and CD8⁺ T cells after CAE-sorting. (FIG. 8B) Representative imaging flow cytometry images of CAE-sorted C1-multimer⁺ apoptotic (left) and EV⁺ CD4 and CD8 T cells are shown. Scale bars=7 μm.

FIG. 9: C1-Tetramers stain CD41⁺ thrombocytes. Whole EDTA-blood sample from a lymphoma patient was stained with C1-multimer (SA-AF647) and CD41, a marker for thrombocytes. Left dot plots show gating of single cells based on the area and aspect ratio of the brightfield signal. Right dot plot was gated on single cells and shows expression of CD41 and surface exposure of PS (by staining with C1-tetramer AF647). Images show CD41⁺C1-Tetramer⁺ aggregated platelets. Scale bars=7 μm.

FIGS. 10A and 10B: Magnetic particle—biotin MFG-E8 C1 Multimer can be utilized to remove dead cells from cell suspensions. Mouse splenocytes were prepared and stained with multimer (biotin-MFG-E8 C1 magnetic particle). Cells were either untreated (before depletion) (FIG. 10A), or treated (after depletion) (FIG. 10B) of wanted cells. GATE I: viability dye+ dead cells; GATE II: viability dye− live cells; GATE III: viability dye− live cells; and GATE IV: viability dye+ dead cells. Numbers indicate percentages in the respective gates. (A) Analysis of cells before depletion by multimer-particles using the gates I, II, III and IV. (B) Analysis of cells after depletion by multimer-particles using gates I, II, III and IV.

FIGS. 11A and 11B: Microbubble—biotin MFGE-8 C1 Multimer can be utilized to remove dead cells from cell suspensions. Mouse splenocytes were prepared and stained with multimer (biotin-MFG-E8 C1 microbubble). Cells were either untreated (before depletion) (FIG. 11A), or treated (after depletion) (FIG. 11B) of wanted cells. GATE I: viability dye+-dead cells; GATE II: viability dye− live cells; GATE III: viability dye− live cells; and GATE IV: viability dye+ dead cells. Numbers indicate percentages in the respective gates. (FIG. 11A) Analysis of cells before depletion by multimer microbubbles using the gates I, II, III and IV. (FIG. 11B) Analysis of cells after depletion by multimer microbubbles using gates I, II, III and IV.

FIG. 12 depicts the ability of exemplary MFGE-8 C1 Multimers to improve single cell sequencing data quality. Fresh isolated PBMCs containing 98% live cells (sample A), or samples containing PBMCs spiked with 40% apoptosis induced heat shock treated human PBMCs (Sample B), were stained with TotalSeq™ TBNK lineage antibodies. One sample set containing PBMCs spiked with heat shock treated PBMCs was incubated with exemplary multimers (biotin-MFG-E8 C1 magnetic particle) for 15 minutes, followed by one wash, and separated using a magnetic force (Sample C). All samples were collected and pooled for scRNAseq sequencing.

### DETAILED DESCRIPTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments described throughout the specification should not be construed to limit the present invention to only the explicitly described embodiments. This description should be understood to support and encompass embodiments which combine the explicitly described embodiments with any number of the disclosed and/or preferred elements. Furthermore, any permutations and combinations of all elements described herein should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e. the subject-matter consists in the inclusion of a stated member, integer or step or group of members, integers or steps. When used herein the term “comprising” can be substituted with the term “containing” or “including” or sometimes when used herein with the term “having”. When used herein “consisting of” excludes any element, step, or ingredient not specified.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Unless otherwise indicated, the term “at least” preceding a series of elements is to be understood to refer to every element in the series. The term “at least two” refers to two, three or more such as four, five, six, seven, eight, nine, ten and more. Those skilled in the art will recognize, or be able to ascertain using no more than routine experimentation, many equivalents to the specific embodiments of the invention described herein. Such equivalents are intended to be encompassed by the present invention.

The term “less than” or in turn “more than” does not include the concrete number.

The term “and/or” wherever used herein includes the meaning of “and”, “or” and “all or any other combination of the elements connected by said term”.

When used herein “consisting of” excludes any element, step, or ingredient not specified in the claim element. When used herein, “consisting essentially of” does not exclude materials or steps that do not materially affect the basic and novel characteristics of the claim.

The term “including” means “including but not limited to”. “Including” and “including but not limited to” are used interchangeably.

The term “about” means plus or minus 20%, preferably plus or minus 10%, more preferably plus or minus 5%, most preferably plus or minus 1%.

Throughout the description and claims of this specification, the singular encompasses the plural unless the context otherwise requires. In particular, where the indefinite article is used, the specification is to be understood as contemplating plurality as well as singularity, unless the context requires otherwise.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be gained from the examples, offered for illustrative purposes only. The examples are not intended to limit the scope of the present invention in any way.

### Phosphatidylserine (PS) Binding Agent

The present invention refers to a phosphatidylserine (PS) binding agent, which comprises one or more (e.g., at least two) isolated C-domains of a milk fat globule-EGF factor 8 (MFG-E8) protein. In some embodiments, the PS binding agent comprises one or more isolated C-domains of a milk fat globule-EGF factor 8 (MFG-E8) protein capable of binding PS. In some embodiments, the PS binding agent comprises one or more isolated C-domains of a milk fat globule-EGF factor 8 (MFG-E8) protein comprising PS binding activity. A PS binding agent is any molecule capable of binding PS, thus having (PS)-binding activity. The term “phosphatidylserine (PS)-binding activity” relates to the ability of a molecule to bind to PS. In the context of the present invention a PS binding agent is understood to be capable of binding PS exposed on the outer leaflet of the cell membrane, i.e. extracellular PS such as PS exposed on dead or dying cells or cell debris, or exposed by extracellular cell-associates vesicles or even exposed by activated thrombocytes as described elsewhere herein. In some embodiments, such PS binding agent of the present invention can be a multimer due to the fact that it comprises at least two C-domains of the MFG-E8 protein linked to each other via multimer assembly as described elsewhere herein. Thus, in some embodiments, the PS binding agent of the present invention comprising at least two C-domains of the MFG-E8 protein as defined elsewhere herein is multimeric. In certain embodiments, the PS binding agent as described herein comprises one C-domain of the MFG-E8 protein. In certain embodiments, the PS binding agent as described herein comprises 2, 3, 4, 5, 6, 7, 8, 9, 10, 20, 30, 40, 50, 60, 70, 80, 90, or 100 or more C-domains of the MFG-E8 protein. Said PS binding agent of the present invention comprising one or more (e.g., at least two) C-domains as defined elsewhere herein does not have a RGD-binding activity as the wild type (WT) MFG-E8 protein.

The term “MFG-E8” refers to the milk fat globule-EGF factor 8 protein, a secreted protein found in vertebrates, including mammals as well as birds. It is a membrane glycoprotein that possesses a phosphatidylserine (PS)-binding activity as well as an RGD-binding activity and, thus, promotes phagocytosis of dying cells. MFG-E8 has also been implicated in wound healing, autoimmune disease, and cancer. MFG-E8 can be further processed to form a smaller cleavage product, medin, which comprises the major protein component of aortic medial amyloid (AMA). Alternative splicing has been reported to result in multiple transcript variants. The term “MFG-E8 protein”, as used herein, includes the full-length MFG-E8 protein, as well as variants thereof. The full-length human MFG-E8 protein is shown in SEQ ID NO: 3 having the Uniprot accession number Q08431 and the full length mouse MFG-E8 protein is shown in SEQ ID NO: 4 having the Uniprot accession number P21956. Variants of the MFG-E8 protein include e.g. a MFG-E8 mutein or an isoform of the MFG-E8 protein, wherein said variants have to have a phosphatidylserine (PS)-binding activity.

As used herein, the term “mutein” refers to a protein having an amino acid sequence that differs from the amino acid sequence of a naturally occurring MFG-E8 protein. Said difference in the amino acid sequence can e.g. be due to a substitution, an addition, an inversion, an insertion and/or a deletion. MFG-E8 “isoforms” are variants that are formed by alternative splicing. The nucleic acid sequences encoding such MFG-E8 variants in accordance with the present invention can be prepared by known methods, such as e.g. by site-directed mutagenesis techniques, high throughput mutagenesis, DNA shuffling, or protein evolution techniques.

Binding to phosphatidylserine is ensured by the presence of the respective PS-binding domains, which in the mouse are for example present at positions 148 to 303 (C1-domain) and at positions 308 to 463 (C2-domain) corresponding to the sequence position of the MFG-E8 protein shown in SEQ ID NO: 4. The respective mouse PS-binding domain is shown in SEQ ID NO: 5. In the human MFG-E8 protein, the respective PS-binding domains are located at positions 70 to 225 (C1-domain) and at positions 230 to 387 (C2-domain) corresponding to the sequence position of the MFG-E8 protein shown in SEQ ID NO: 3.

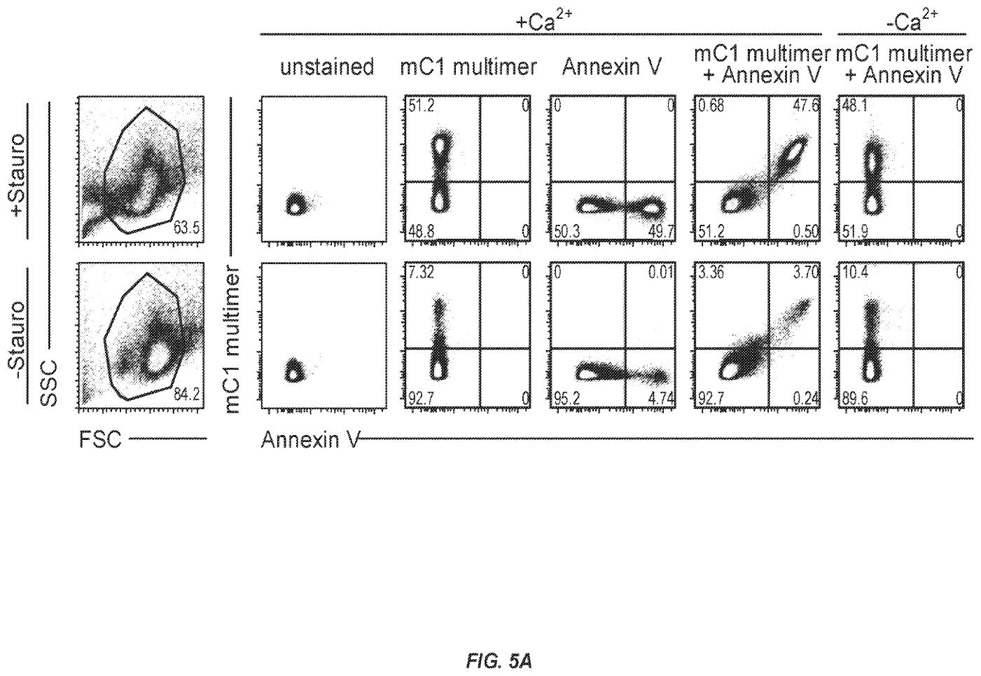

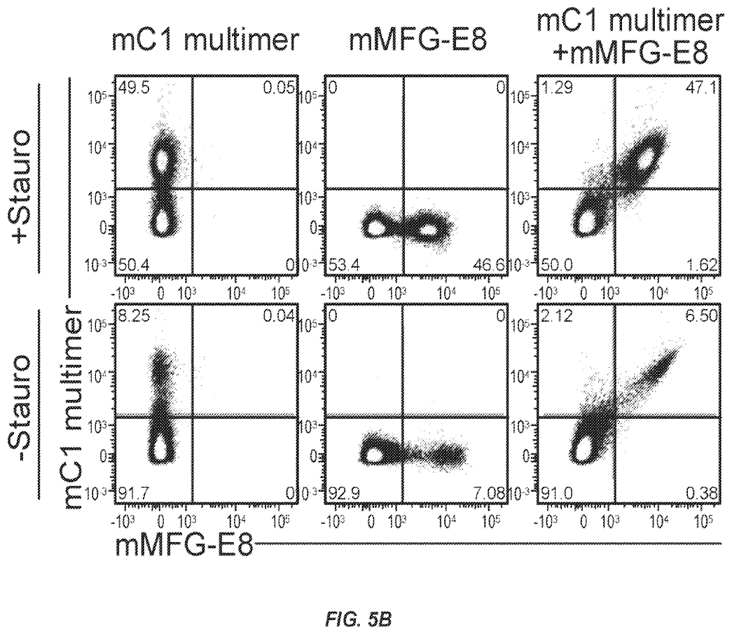

Whether an agent is considered as a PS binding agent/having a phosphatidylserine (PS)-binding activity can be determined by using for example a FACS assay detecting the PS-binding activity of said agent as it is known in the art. Thereby, PS positive dead or dying cells, activated thrombocytes and/or cell debris can be used in such assay, where the number of stained cells may be compared to a reference reagent (e.g. Annexin V) (see FIGS. 5A and 5B). In accordance with the present invention, an agent is considered a PS binding agent/an agent having a phosphatidylserine (PS)-binding activity if it has at least 10%, such as e.g. at least 25%, preferably at least 50%, more preferred at least 75%, even more preferred at least 80%, even more preferred as at least 90%, even more preferred at least 95% and most preferred at least 98% of the phosphatidylserine (PS)-binding activity as observed for the human MFG-E8 protein shown in SEQ ID NO: 3 or the mouse MFG-E8 protein shown in SEQ ID NO: 4 when tested by the same method of determining the respective activity as described above. Preferably, the above recited values are relative percentages of phosphatidylserine (PS)-binding activity as observed for the human MFG-E8 protein shown in SEQ ID NO: 3.

As already mentioned herein, murine lactadherin comprises two N-terminal epidermal growth factor homology domains (EGF1 and EGF2), and the C-terminal region comprises two C-domains, namely the F5/8 type C1 and F5/8 type C2 domains (short: C1 and C2 domains), which share homology with the lipid-binding “C”-terminal, twice repeated domain of about 150 amino acids of blood coagulation factor V and factor VIII. However, the PS binding agent of the present invention comprising one or more (e.g., at least two) C-domains of the MFG-E8 protein does not comprise any EGF domain nor the RGD-binding motif. Nonetheless, the term “comprising”, as used in this respect denotes that further components or molecules can be included in addition to the specifically recited C-domains, such as labels as described elsewhere herein. For example, in those embodiments where the PS binding agent of the present invention includes more than the recited C-domains, said additional molecules, preferably at the C- or N-terminus of the comprised C-domains, may include for example sequences introduced for purification, typically peptide sequences that confer on the resulting PS binding agent an affinity to certain chromatography column materials. Typical examples for such sequences include, without being limiting, tags such as an oligohistidine-tag, a Sirep-tag, a FLAG-tag, a glutathione S-transferase (such as GST-SUMO3 tag), a maltose-binding protein or the albumin-binding domain of protein G. However, this term also encompasses that the claimed subject-matter consists of exactly the recited C-domains.

In accordance with the present invention, it is envisaged that the one or more (e.g., at least two) C-domains of a MFG-E8 protein comprised by the PS binding agent described herein are any one of a C1-domain of a MFG-E8 protein, a C2-domain of a MFG-E8 protein or a functional fragment thereof. In addition, said C-domains may be comprised by a proteinaceous entity of the PS binding agent of the present invention. Thus, a PS binding agent comprising a proteinaceous entity comprising one or more (e.g., at least two) C-domains of a MFG-E8 protein as defined elsewhere herein is also comprised by the present invention. In the context of the present invention, the “proteinaceous entity” refers to an entity/unit which comprises said one or more (e.g., at least two) C-domains as defined elsewhere herein. Since said one or more (e.g., at least two) C-domains of a MFG-E8 protein do not refer to said wild type (WT) MFG-E8 protein as defined herein, said entity/unit which comprises said domains is proteinaceous. The term “proteinaceous” may also refer to “protein-like”.

In a preferred embodiment, the PS binding agent of the present invention comprises one or more (e.g., at least two) C-domains of a MFG-E8 protein as defined elsewhere herein. In this respect it is envisaged that the PS binding agent of the present invention comprises two C2-domains (C2-C2) of a MFG-E8 or two C1-domains (C1-C1) of a MFG-E8 protein. In an even more preferred embodiment, the PS binding agent of the present invention comprises at least three C-domains of a MFG-E8 protein as defined elsewhere herein. In this respect it is envisaged that the PS binding agent of the present invention comprises three C2-domains (C2-C2-C2) of a MFG-E8 or three C1-domains (C1-C1-C1) of a MFG-E8 protein. Still more preferred, the PS binding agent of the present invention comprises four C-domains of a MFG-E8 protein as defined elsewhere herein. Thus, it is preferred that the PS binding agent of the present invention comprises four C2-domains of a MFG-E8 protein such as C2-C2-C2-C2. Mostly preferred by the present invention is the PS binding agent comprising four C1-domains of a MFG-E8 protein such as C1-C1-C1-C1.

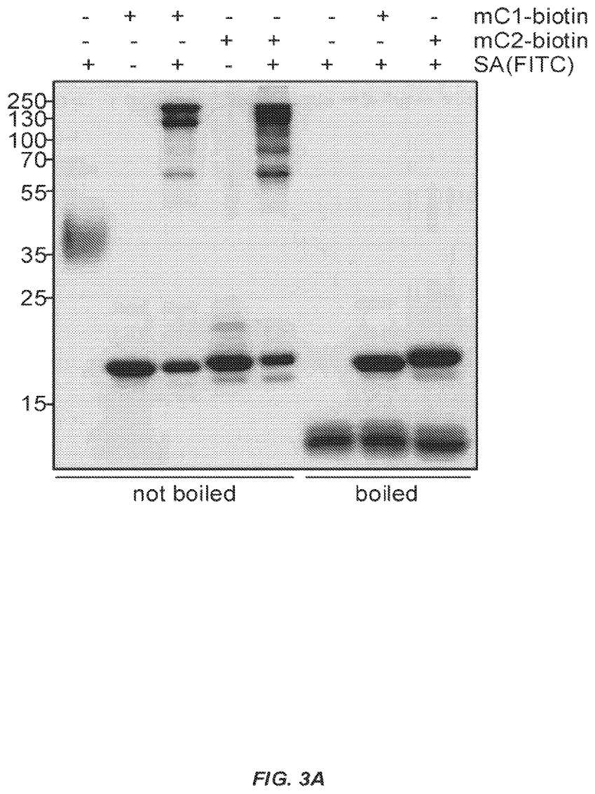

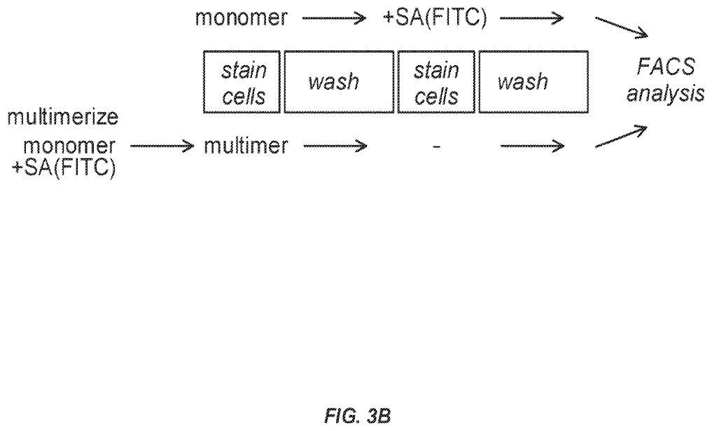

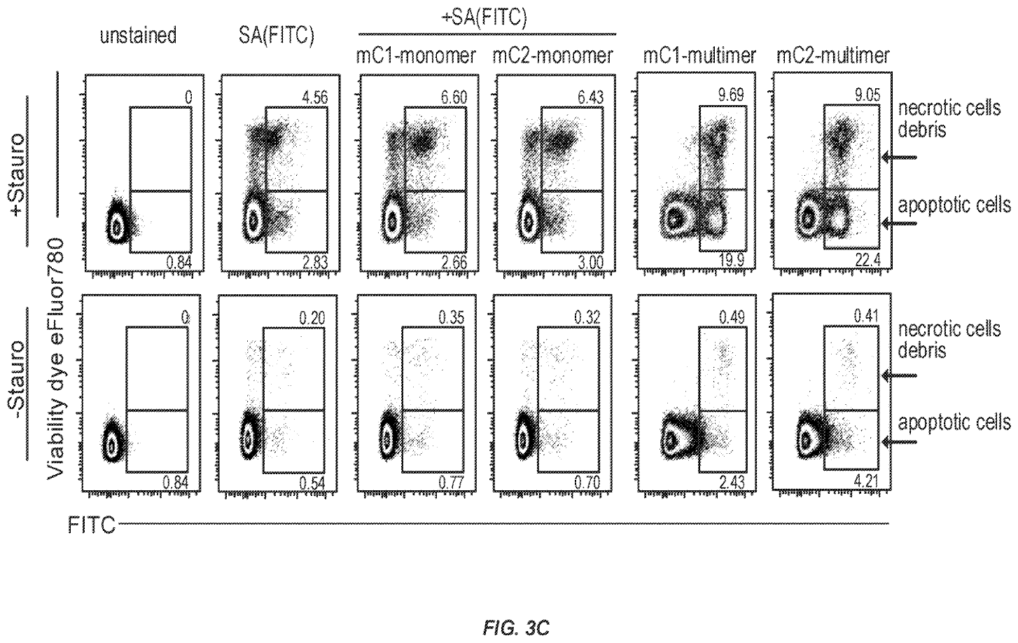

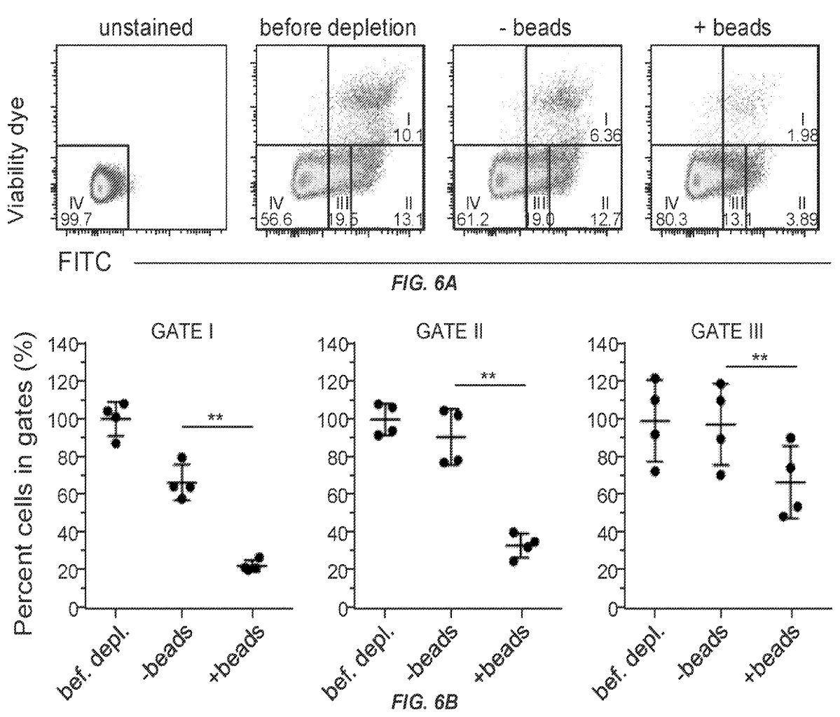

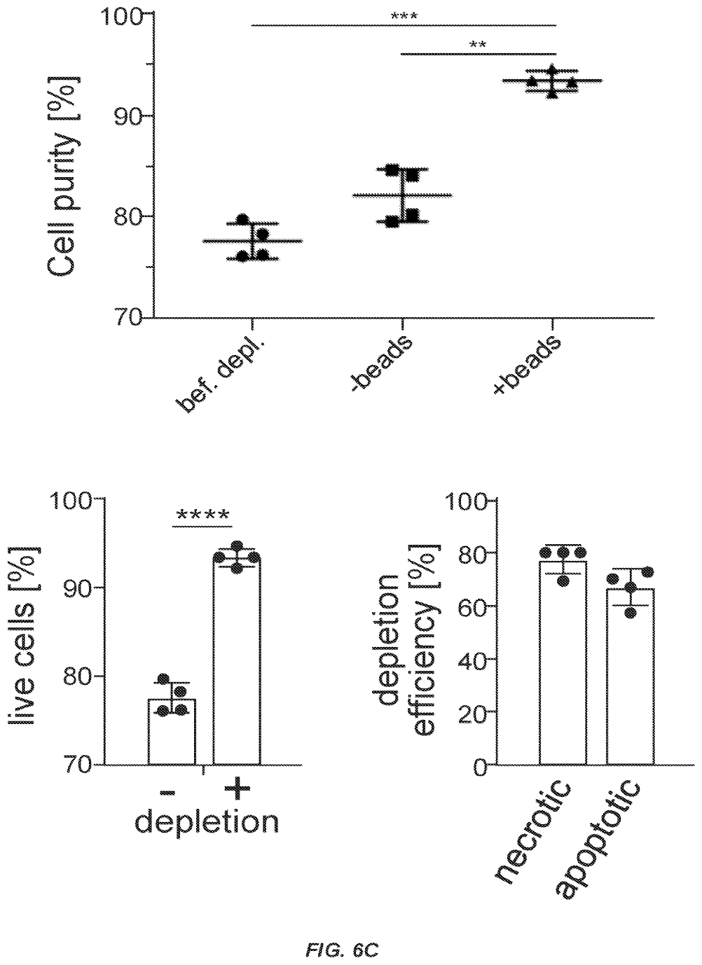

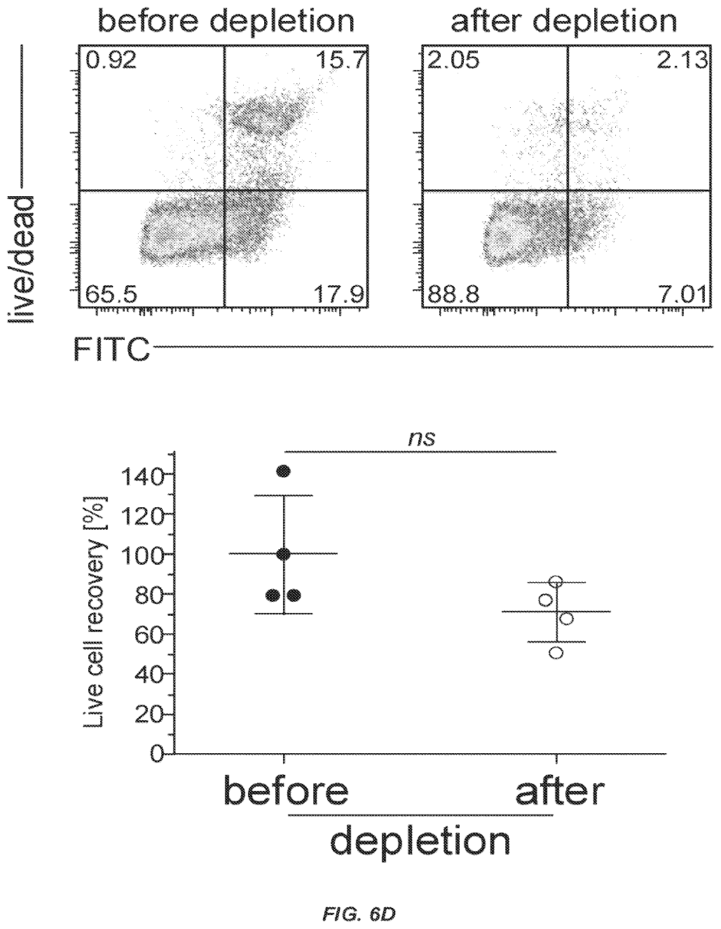

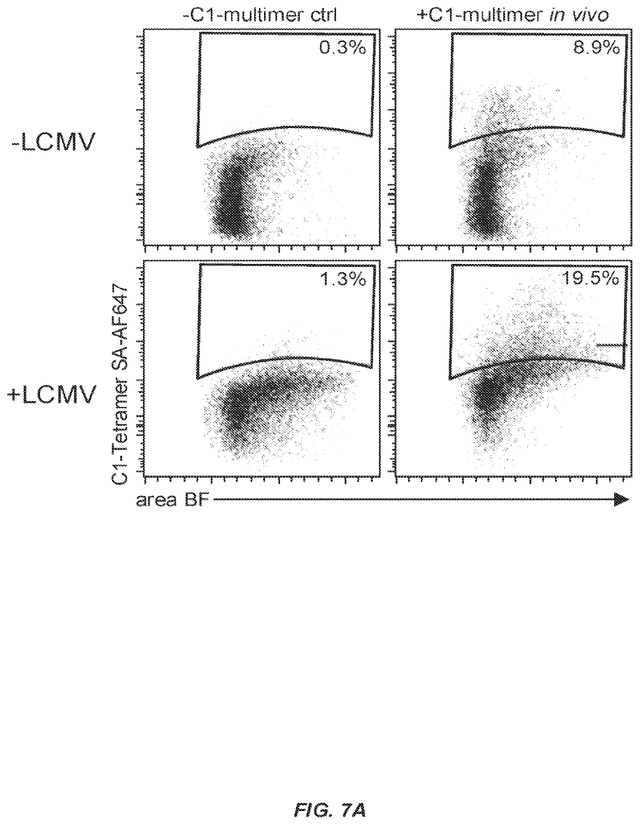

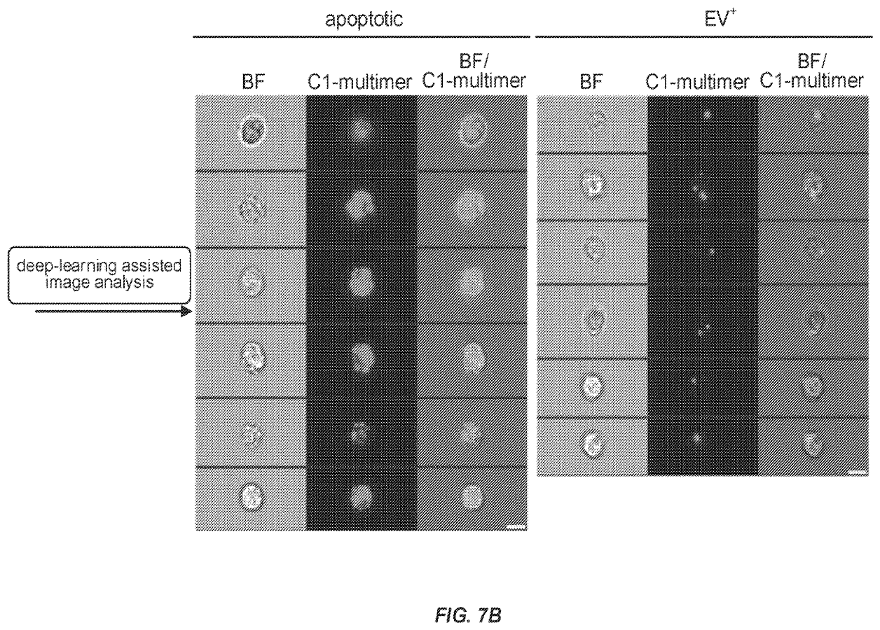

As described elsewhere herein, the inventors of the present invention impressively demonstrated that C1-multimers show increased avidity to apoptotic and necrotic cells when compared to C2-multimers, while monomers of both single domains did not bind PS on dead or dying cells, cell debris, activated thrombocytes and/or EVs (see FIGS. 3A-3C). Hence, the C1-multimers of the present invention prove to be an effective and buffer-independent alternative to Annexin V, an already established PS-binding molecule. Thus, in some embodiments, the one or more (e.g., at least two) C-domains of the PS binding agent of the present invention are a C1-domain of a MFG-E8 protein. In other words, a PS binding agent comprising one or more (e.g., at least two) C1-domains of a MFG-E8 protein (such as at least two C1-C1), is also encompassed herein and preferred. It is particularly preferred in this respect that the PS binding agent comprising 4 C1-domains of a MFG-E8 protein. For research and diagnostic purposes, these C1-multimers represent a platform for various in vitro applications. For example, C1-multimers of the present invention can be used for the depletion of apoptotic and/or necrotic cells, cell debris from cell suspensions for the enrichment of living cells for research and development purposes (see FIGS. 6A-6D, 10A, 10B, 11A, and 11B). Furthermore, these C1-multimers can be used to reliably detect cell-associated PS positive vesicles in vivo (see FIGS. 7A and 7B), which are often associated with cancer, inflammation, infection and autoimmunity, the detection of which was difficult so far.

A functional fragment of the C1-domain or the C2-domain of a MFG-E8 protein as used in the context of the present invention refers to functional equivalent parts of the C1- or C2-domain of the MFG-E8 protein having the same functional characteristics as the C1- or C2-domain of a MFG-E8 protein. Hence, said functional fragment, no matter what length it has, still comprises the PS binding domain in order to be considered as a PS binding agent (agent capable of binding PS, thus having (PS)-binding activity). Accordingly, said functional fragment have the same phosphatidylserine (PS)-binding activity when compared to the intact C1- or C2-domain defined herein. Further, said functional fragments can be equally linked to each other via multimer assembly, i.e. form PS binding multimers, as described elsewhere herein for complete C1- and C2-domains of the MFG-E8 protein. Preferably, a functional fragment of the C1-domain and/or the C2-domain of a MFG-E8 used in the context of the present invention may refer to having at least 5, at least 6, at least 7, at least 8, at least 9, at least 10, at least 11, at least 12, at least 13, at least 15, at least 20, at least 25, at least 30, at least 35, at least 40, at least 45, at least 50 amino acids of a MFG-E8 protein. Preferably, a functional fragment of the C1-domain and/or the C2-domain of a MFG-E8 used in the context of the present invention may refer to at least 5 amino acids of the MFG-E8 protein as defined herein which are responsible for PS binding as it is described in Barth et al. 2020, Nature Communications, 11(1), 1-14 and/or Zhen et al. (2011), Journal of the American Chemical Society, 133(39), 15280-15283, even more preferably arranged as a cyclic molecule (peptide). Here, from the crystal structure of MFG-E8 protein the PS binding amino acids may be determined and then as a small cyclic peptide only these particular amino acids may be cloned side by side forming the functional fragment of the C1-domain and/or the C2-domain of a MFG-E8 used in the context of the present invention. Thus, also a PS binding agent comprising one or more (e.g., at least two) fragments of the C1- or C2-domain of a MFG-E8 protein can be understood as a PS binding agent in the context of the present invention. Further preferred is a PS binding agent comprising four fragments of the C1- or the C2-domain of a MFG-E8 as defined herein.

Further, the present invention also refers to a PS binding agent as defined herein, wherein at least one of said C-domains is a C1-domain of a MFG-E8 protein and at least one of said C-domains is a C2-domain of a MFG-E8 protein. Hence, the PS binding agents of the present invention also refer to a multimer comprising a mixture of the C1- and the C2-domain(s) of a MFG-E8 protein. When the multimer comprises at least one C1-domain and at least one C2-domain, it refers to a multimer comprising one, two, three, four or more C1-domains and one, two, three, four or more C2-domains such as for example a PS binding agent (multimer) comprising, when having for example only two C-domains, C1-C2 (such as comprising one C1- and one C2-domain). When having for example three C-domains, the PS binding agent (multimer) may comprise C1-C2-C1 (such as comprising two C1-domains), C1-C2-C2 (such as comprising two C2-domains). When having for example four C-domains, the PS binding agent (multimer) may comprise C1-C2-C2-C2 (such as comprising one C1-domain), C2-C1-C1-C1 (such as comprising one C2-domain), C1-C2-C2-C1 (such as comprising two C1-domains), C2-C1-C1-C2 (such as comprising two C2-domains), C2-C1-C1-C1 (such as comprising three C1-domains), C1-C2-C2-C2 (such as comprising three C2-domains) and so on.

In the context of the present invention, a C1-domain of a MFG-E8 of the PS binding agent of the disclosure may have at least about 60% (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) with the amino acid sequence of the murine C1-domain (SEQ ID NO: 1). Alternatively or simultaneously, a C2-domain of a MFG-E8 of the PS binding agent of the disclosure may have also at least about 60% (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) with the amino acid sequence of the murine C2-domain (SEQ ID NO: 2).

Any types and numbers of mutations, including substitutions, deletions, and insertions as known to a person skilled in the art, are envisaged as long as the provided PS binding agent retains its capability to bind its given target PS, preferably on dead or dying cells, cell debris, activated thrombocytes and/or EVs and said C1-domain of the PS binding agent has a sequence identity that it is at least about 60%, such as at least about 65%, at least about 70%, at least about 75%, at least about 80%, at least about 85%, at least about 90%, at least about 95%, including at least about 96%, at least about 97%, at least about 98%, at least about 99%, at least about 99.4% or even 100% identity to the amino acid sequence of the reference (wild-type) murine C1-domain (SEQ ID NO: 1). Alternatively or simultaneously, any types and numbers of mutations, including substitutions, deletions, and insertions as known to a person skilled in the art, are envisaged as long as the provided PS binding agent retains its capability to bind its given target PS, preferably on dead or dying cells, cell debris, activated thrombocytes and/or EVs and said C2-domain of the PS binding agent has a sequence identity that it is at least about 60%, such as at least about 65%, at least about 70%, at least about 75%, at least about 80%, at least about 85%, at least about 90%, at least about 95%, including at least about 96%, at least about 97%, at least about 98%, at least about 99%, at least about 99.4% or even 100% identity to the amino acid sequence of the reference (wild-type) murine C2-domain (SEQ ID NO: 2).

Thus, the present invention may further comprise the PS binding agent as defined elsewhere herein, wherein said C1-domain comprises an amino acid sequence having at least about 60%, at least about 65%, at least about 70%, at least about 75%, at least about 80%, at least about 85%, at least about 90%, at least about 95%, including at least about 96%, 97%, 98%, 99% or even 100% sequence identity with an amino acid sequence of SEQ ID NO: 1. Also comprised by the present invention is the PS binding agent as defined elsewhere herein, wherein said C2-domain comprises an amino acid sequence having at least about 60%, at least about 65%, at least about 70%, at least about 75%, at least about 80%, at least about 85%, at least about 90%, at least about 95%, including at least about 96%, 97%, 98%, 99% or even 100% sequence identity with an amino acid sequence of SEQ ID NO: 2. In this embodiment the term “at least about” includes each single %-value starting from 60% to 100% sequence identity, such as at least about 60%, 61%, 62%, 63%, 64%, 65%, 66%, 67%, 68%. 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or even 100% sequence identity with an amino acid sequence of SEQ ID NO: 1 or 2.

Even more preferably, the present invention may further comprise the PS binding agent as defined elsewhere herein, wherein said C1-domain has the amino acid sequence of SEQ ID NO: 1. SEQ ID NO: 1 refers to the wild-type murine C1-domain of MFG-E8 protein. Also even more preferably, the present invention may further comprise the PS binding agent as defined elsewhere herein, wherein said C2-domain has the amino acid sequence of SEQ ID NO: 2. SEQ ID NO: 2 refers to the wild-type murine C2-domain of MFG-E8 protein.

In a preferred embodiment of the present invention, said one or more (e.g., at least two) C-domains comprised by the PS binding agent as defined elsewhere herein are biotinylated. Meaning each C-domain as defined herein of the one or more (e.g., at least two) C-domains of the PS binding agent is biotinylated. It may also be comprised herein that the PS binding agent as used herein (which comprises the one or more (e.g., at least two) C-domains) is biotinylated. Biotinylation of target molecules and substrates is well known, for example, a large number of biotinylation agents are known, including amine-reactive and thiol-reactive agents, for the biotinylation of proteins, nucleic acids, carbohydrates, carboxylic acids; see chapter 4, Molecular Probes Catalog, Haugland, 6th Ed. 1996, hereby incorporated by reference. Biotinylation is preferably performed by sortase A and a Biotin-containing peptide or a biotin ligase, such as BirA. Thus, according to the present invention said one or more (e.g., at least two) C-domains comprised by the PS binding domain as defined elsewhere herein may be biotinylated with a biotin-containing peptide using sortase A or a biotin ligase such as BirA. In this context, it should be noted that biotinylation performed by sortase A and a Biotin-containing peptide or a biotin ligase, such as BirA occurs on the one or more (e.g., at least two) monomeric C-domains of the present invention. Only after the biotinylation of the two or more monomeric C-domains, said two or more C-domains are multimerized as defined elsewhere herein to form the PS binding agent.

Sortase enzymes are prokaryotic enzymes that have been used extensively for protein engineering and antibody modifications. The use of sortase and its recognition sequence is thus well known in the art and has been described, e.g. in Alt et al. (Angew. Chem. Int. Ed. 2015, 54, 7515-7519). The use of sortase and its recognition sequence also allows for site-specific addition of biotin. Applied to the present invention, the PS binding agent can for example be fused to a sortase A recognition sequence. In the context of the present invention, the sortase A used to biotinylate said C-domains of the PS binding agent may be either the Ca²⁺-dependent sortase A from Staphylococcus aureus or sortase A from Staphylococcus pyogenes, which is Ca²⁺-independent. Preferably, in the present invention sortase A from S. aureus is used to biotinylate said monomeric C-domains, which will then be later on multimerized as defined elsewhere herein to form the PS binding agent of the present invention which then comprises said two or more biotinylated C-domains.

In the specific instance of C-terminal labeling/C-terminal biotinylation of a protein to be labeled, said protein, in the context of the present invention the one or more (e.g., at least two) C-domains of the PS binding agent, is modified at its C-terminus with the LPXTG (SEQ ID NO: 7) or LPXTA (SEQ ID NO: 8) (X being any amino acid) sortase-recognition sequence followed by a oligohistidine-tag (His₆-tag) and incubated with S. aureus sortase A or S. pyogenes sortase A. Sortase then cleaves the threonine-glycine bond and via its active site cysteine residue forms an acyl intermediate with threonine in the protein. The addition of a biotin-containing peptide bond comprising a series of N-terminal glycine residues and a functional moiety of choice resolves the intermediate, thus regenerating the active site cysteine (HS) on sortase and ligating the biotin-containing peptide probe to the C-terminus of the protein (Guimaraes et al. 2013, Nat Protoc, Vol. 8, No. 9). Said biotinylation reaction is further defined in the Example section.

Besides C-terminal labeling there is also N-terminal labeling of the protein to be labeled using sortase A as it is described by Theile et al. 2013, Nat Protoc, 8(9): 1800-1807. In this particular reaction the protein to be labeled is engineered with an exposed stretch of glycines or alanines at its N-terminus when using for example sortase A from S. aureus or S. pyogenes, respectively. A biotin-containing peptide with three glycines (GGG) at its N-terminus is then added to the reaction together with sortase and the C1 or C2 domain with the sortase recognition sequence LPXTG as depicted in SEQ ID NO: 7 or the sortase recognition sequences LPXTA as depicted in SEQ ID NO: 8. Sortase A cleaves between the Thr and Gly/Ala residues, forming a thioester intermediate with the peptide probe. Nucleophilic attack by the N-terminally modified protein of interest resolves the intermediate, resulting in the formation of a covalent bond between the peptide probe and the N-terminus of the PS binding agent. Alternatively, depsi-peptides can be used for N-terminal labeling. Depsi-peptides feature an ester linkage between the threonine and glycine, instead of an amide peptide bond to yield a more effective leaving group. By using depsi-peptides, the probe concentration in the reaction can be lowered while maintaining yields.

The one or more (e.g., at least two) C-domains of the PS binding agent of the present invention may thus be biotinylated N- or C-terminally, preferably C-terminally. Also comprised herein is the one or more (e.g., at least two) C-domains of the PS binding agent as defined throughout the present invention being biotinylated N- or C-terminally, preferably C-terminally, using sortase A derived from S. aureus or S. pyogenes, preferably derived from S. aureus, or using a biotin ligase as defined elsewhere herein.

Such biotin ligase (also called biotin protein ligase, BPL) activates biotin to form biotinyl 5′ adenylate and transfers the biotin to biotin-accepting proteins. In detail, in the first partial reaction, a BPL catalyzes the synthesis of biotinoyl-AMP (bio-5′-AMP, which is also called biotinyl-adenylate) from ATP and biotin. The enzyme then sequesters bio-5′-AMP in the active site until the second partial reaction proceeds. In the second partial reaction, the nucleophilic ε-amino group of the target lysine residue of a biotin-accepting domain attacks the mixed anhydride of the bio-5′-AMP bound within the BPL active site to form an amide bond between biotin and the lysine side chain that remains intact for the life of the protein (Choi-Rhee et al. 2004, Protein Sci, 13(11): 3043-3050). The natural substrate of BirA is the Biotin Carboxyl Carrier Protein (BCCP). However, based on phage display selection AviTag (also known as the Acceptor Peptide, AP) may also be used. In this case a linker as depicted in SEQ ID NO: 6 can be used before the AviTag. It is also superior to BCCP as a BirA substrate. A suitable biotin ligase is for example the enzyme “E. coli repressor of biotin biosynthesis” (BirA), which is capable of transferring biotin to a unique lysine residue in the recognition sequence within the acceptor protein, which is in the context of the present invention the one or more (e.g., at least two) C-domains of the PS binding agent. A detailed protocol for biotinylating using BirA is disclosed by Fairhead and Howarth (2015), Methods in molecular biology 1266, 171-184, which is incorporated by reference herewith. BirA from other species other than from E. coli can also be used for biotinylating said one or more (e.g., at least two) C-domains of the PS binding agent of the present invention as defined elsewhere herein.

In the context of the present invention, said one or more (e.g., at least two) C-domains comprised by the PS binding agent as defined elsewhere may be directly or indirectly linked.

A “direct linkage” or “linking directly” as used throughout the present invention refers to linking said two or more C-domains of the PS binding agent as defined elsewhere herein or linking said two or more C-domains to each other, thereby forming the PS binding agent comprising two or more C-domains of the present invention without any linker or additional molecule. One could link said two or more C-domains genetically, thereby simply arranging said two or more C-domains one after the other and thus expressing it in a host cell, such as bacteria.

A “indirect linkage” or “linking indirectly” refers to linking said at least two C-domains of the PS binding agent as defined elsewhere herein or linking said at least two C-domains to each other in the context of the kit and/or of the method of producing as defined elsewhere herein via an additional molecule capable of linking said C-domains, thereby forming the PS binding agent comprising at least two C-domains of the present invention. Hence, the PS binding agent of the present invention may comprise an additional molecule capable of linking the C-domains as defined herein. Preferably, said additional molecule is streptavidin, avidin or neutravidin, wherein streptavidin is particularly preferred. Accordingly, said “indirect linkage” comprises the use of avidin, streptavidin (SA), or neutravidin, even more preferably the use of SA. Also it should be noted that said multimeric PS binding agent can be assembled with any modified avidin, streptavidin or neutravidin such as modified by a fluorescent dye, enzyme, antigen or other labels as defined elsewhere herein.

Streptavidin (SA) is preferably used as an indirect linkage of the C-domains of the PS binding agent of the present invention. Thus, the present invention may comprise that the at least two C-domains of the PS binding agent as defined elsewhere herein are indirectly linked via streptavidin. Streptavidin as indirect linkage molecule of said at least two C-domains or said C-domains that are linked to each other in the context of the kit and/or of the method of producing binds to biotin with a K^{d} of 4×10⁻¹⁴ M (Green 1990, Methods Enzymol; 184:51-67). Streptavidin-biotin binding is rapid, specific and can still occur under conditions where most other proteins have denatured. It has a maximum of four binding sites, thus a PS binding agent comprising four C-domains as defined elsewhere herein is preferred, wherein each binding site of SA is occupied by one biotinylated C-domain as defined herein via biotin-streptavidin binding.

Streptavidin used when indirectly linking said at least two C-domains of the PS binding agent or when used in context of the kit/method of producing, may be labelled, preferably with a fluorophore or an enzyme for producing bioluminescence. Suitable fluorophores are known to the person skilled in the art and defined elsewhere herein. A suitable enzyme refers to but is not limited to luciferase. Commercially available fluorophore-SA conjugates can be easily exchanged in the multimer of the present invention for microscopic imaging, flow cytometry and flow microscopy applications in vitro. In addition, luciferase-fused SA would allow for example drug screens inducing cell death in a microplate-reader. These embodiments with regard to SA may be applicable to avidin and/or neutravidin as well.

When a linker is used for indirect linkage of the C-domains of the PS binding agent or in the context of the present invention, such as the kit/method of producing of the present invention as well as for linking said PS binding agent to a label as defined elsewhere herein, said linker is preferably 1 to 100 amino acids in length. More preferably, the linker is 5 to 50 amino acids in length, such as e.g. 10 to 30 amino acids in length and even more preferably, the linker is 12 to 25 amino acids in length. Even more preferably, the linker is 15 to 20 amino acids in length, such as 15 to 17 amino acids, and most preferably, the linker has a length of 15 amino acids or a length of 17 amino acids. It is preferred that the linker molecule is a linear or a helical linker, even more preferably the linker is a helical linker. It is further preferred that the linker is a flexible linker using e.g. the amino acids glycine and/or serine. In a particularly preferred embodiment of the invention, between 50% and 100%, particularly between 60% and 100%, particularly between 70% and 100%, particularly between 80% and 100%, particularly between 90% and 100%, and especially 100% of the amino acid residues of the linker molecule are glycine and serine residues, preferably forming an alpha-helix structure.

The length and sequence of a suitable linker depends on the composition of the respective PS binding agent. Methods to test the suitability of different linkers are well known in the art and include e.g. the comparison of the protein stability or the production yield of the PS binding agent of the invention comprising different linkers to the respective PS binding agent without a further molecule fused thereto. Furthermore, and in accordance with the present invention, it has to be ensured that the linker does not interfere with the phosphatidylserine (PS)-binding activity of the resulting agent of the invention. For testing whether a linker fulfils this requirement, the phosphatidylserine (PS)-binding activity of the agent of the invention can be determined as described herein above.

The linkage, either directly or indirectly, of the C-domains of the PS binding agent may be covalently. The present invention thus further comprise the PS binding agent as defined elsewhere herein, wherein said C-domains are covalently linked. The term “covalently linked” in the context of the PS binding agent and/or in the context of the method of producing refers to covalent bonds that are typically formed by the sharing of electron pairs between atoms. In accordance with the present invention and when the term “covalently linked” is used, a covalent bond is formed between the at least two C-domains of the PS binding agent of the present invention or between said C-domains used in the method of producing the PS binding agent as defined elsewhere herein by use of a peptide linker of at least 1 amino acid in length as defined above.

In the context of the present invention, the PS binding agent may further comprise a recognition site for site-specific modifications, such as a recognition sequence for enzymatic modifications. Said recognition sequence for enzymatic modification serves as an “adapter”, i.e. it is suitable to connect compounds or molecules of interest at a later stage to the PS binding agent of the invention. Further, the coupling of compounds or molecules that normally cannot be chemically or genetically connected to the PS binding agent can be achieved by this adapter technique. Any recognition sequence may be comprised in said PS binding agent, which is used that an enzyme can recognize said PS binding agent and will then be able to modify said agent enzymatically as it is known to a person skilled. In particular, said recognition sequence of the PS binding agent is comprised in all of the one or more (e.g., at least two) C-domains of said PS binding agent. Said sequence may thus be comprised by the C1-domain, by the C2-domain of a MFG-E8 protein or by a fragment thereof as it is defined elsewhere herein. Thus, when referring to said C1- and/or said C2-domain or a fragment thereof comprising an amino acid sequence having at least about 60% identity as defined herein with the amino acid sequence of SEQ ID NO: 1 or 2, said C1- and/or said C2-domain or a fragment thereof may still comprise said recognition sequence as will be defined in the following. Such recognition sequences for enzymatic modifications are well known in the art and have been described e.g. in Voloshchuk et al. (Voloshchuk, N., Liang, D. & Liang, J. F. Sortase A Mediated Protein Modifications and Peptide Conjugations. Curr Drug Discov Technol (2015)).

In a preferred embodiment of the present invention, the PS binding agent as defined elsewhere herein comprises a recognition sequence, wherein the recognition sequence is for a biotin ligase or for a sortase A, preferably wherein the biotin ligase is BirA. When the PS binding agent comprises a recognition sequence for a biotin ligase, preferably for BirA, or for a sortase A, each of the one or more (e.g., at least two) C-domains as defined herein comprise said recognition sequence in order to become biotinylated by said biotin ligase (such as BirA) or by said sortase A.

Suitable recognition sequences for BirA are well known and have been described in the art, e.g. in Fairhead and Howarth (2015), Methods in molecular biology 1266, 171-184. When BirA is used for biotinylation, the PS binding agent may then be designed to comprise a suitable recognition sequence in the C1- and/or C2-domain or in the fragment thereof as disclosed by Fairhead and Howarth 2015. For example, when the PS binding agent is fused to a BirA recognition sequence, the BirA enzyme then catalyzes the site-specific addition of a biotin. This would, for example, allow the detection of the binding agent, and therefore of any dead or dying cells, cell debris, activated thrombocytes and/or extracellular vesicles bound by said binding agent, via biotin-based detection systems. Evidently, these systems can also be used for isolation and/or purification purposes of dead or dying cells, cell debris, activated thrombocytes and/or extracellular vesicles.

When sortase A is used for biotinylating said PS binding agent, in particular said one or more (e.g., at least two) C-domains of said PS binding agent, sortase A from S. aureus or S. pyogenes may be used. Substrates having a LPXTG (SEQ ID NO: 7) recognition sequence (also called motif) are suitable for sortase A derived from S. aureus, and substrates having a LPXTA (SEQ ID NO: 8) recognition motif are suitable for sortase A derived from S. pyogenes (Guimaraes et al. 2013, Nat Protoc, Vol. 8, No. 9). These substrates, and in context of the present invention the PS binding agent, in particular said one or more (e.g., at least two) C-domains of said PS binding agent, can be engineered using standard molecular cloning protocols.

The present invention may further comprise a PS binding agent as defined herein, wherein the recognition sequence for sortase A is LPXTG (SEQ ID NO: 7). Also comprised herein is a PS binding agent as defined elsewhere, wherein said one or more (e.g., at least two) C-domains comprise a recognition sequence for sortase A, wherein said recognition sequence is LPXTA (SEQ ID NO: 8). After the proline in said recognition sequences any amino acid sequence may be used. Usually, said recognition sequence is engineered at the C-terminus of the protein to be modified, with the G or A residue in amide linkage, followed by an affinity purification handle (e.g. His₆). Thus, the PS binding agent of the present invention may comprise a LPXTG (SEQ ID NO: 7) recognition sequence for sortase A at the C-terminal end. It may also be comprised herein that a flexible linker composed of (Gly₄Ser)n preceding said LPXTG (SEQ ID NO: 7) or LPXTA (SEQ ID NO: 8) recognition sequence is used. Preferably, a flexible linker composed of (Gly₁Ser) (GS) preceding said LPXTG (SEQ ID NO: 7) or LPXTA (SEQ ID NO: 8) recognition sequence is used.

It is also within the context of the present invention that sortase B is used for biotinylating said PS binding agent, in particular said one or more (e.g., at least two) C-domains of said PS binding agent as described e.g. in Puorger et al. 2017, Biochemistry, 56, 21, 2641-2650 and Antos et al. 2009, Journal of the American Chemical Society, 131(31): 10800-10801. Substrates having a DXPNTGDE (SEQ ID NO: 9) recognition sequence (also called motif) are suitable for sortase B. After the aspartic acid in said recognition sequences any amino acid sequence may be used. These substrates, and in context of the present invention the PS binding agent, in particular said one or more (e.g., at least two) C-domains of said PS binding agent, can be engineered using standard molecular cloning protocols.

Preferably, in the context of the present invention the recognition sequence for sortase A comprised by the PS binding agent as defined herein is LPETG (SEQ ID NO: 13), even more preferably located at the C-terminal end of said PS binding agent. Also comprised herein is the PS binding agent as defined elsewhere, wherein said one or more (e.g., at least two) C-domains comprise a recognition sequence for sortase A, wherein said recognition sequence is LPETG (SEQ ID NO: 13), preferably located at the C-terminal end.

Also comprised by the present invention is that the PS binding agent may be labeled. Preferably, said label is linked to said additional molecule capable of linking said at least two monomeric C-domains as described elsewhere herein such as streptavidin, avidin or neutravidin. The label may be any one of, but not limited to, a fluorescent label, an enzyme label (such as bioluminescent reporter luciferase), a tag label, a molecule label, which is preferably capable of biotinylating said agent, or a radioactive isotope label known to the person skilled in the art. When the label is a molecule label it may refer to a partner of a binding pair. Such label may then be indirectly detected. By “partner of a binding pair” is meant one of a first and a second moiety, wherein said first and said second moiety have a specific binding affinity for each other. Suitable binding pairs for use in the invention include, but are not limited to, antigens/antibodies (for example, digoxigenin/anti-digoxigenin, dinitrophenyl (DNP)/anti-DNP, dansyl-X-anti-dansyl, Fluorescein/anti-fluorescein, lucifer yellow/anti-lucifer yellow, and rhodamine anti-rhodamine), magnetic beads/magnet and calmodulin binding protein (CBP)/calmodulin.

In the context of the present invention, the label comprised by the PS binding agent as defined herein, specifically linked to said additional molecule capable of linking said at least two monomeric C-domains as described elsewhere herein, may be selected from the group consisting of a tag, a fluorophore, a solid phase carrier, a microbubble, a linker, and an active site probe, i.e. a detectable moiety suitable for the labeling and detection of dead or dying cells, cell debris, activated thrombocytes and/or extracellular vesicles in vitro by methods well known in the art. This allows, for example and in the context of the present invention, the detection of the PS binding agent, and therefore of dead or dying cells, cell debris, activated thrombocytes and/or extracellular vesicles bound by said PS binding agent.

When the label is a tag, the disclosure of the abovementioned should be considered herein. Preferably, when the label is a tag, an oligohistidine-tag (His₆ tag) is used in the PS binding agent as defined elsewhere herein. Said tag, in particular said histidine-tag, is comprised in the one or more (e.g., at least two) C-domains of the PS binding agent. Even more preferably, said tag, in particular said histidine-tag, is comprised C-terminally in the one or more (e.g., at least two) C-domains of the PS binding agent. Most preferably, said tag, in particular said histidine-tag, is comprised C-terminally after the recognition sequence as defined elsewhere herein in the one or more (e.g., at least two) C-domains of the PS binding agent.

When the label is a fluorophore (also called fluorochrome or chromophore) in the present invention it may be any one of a fluorescent dye such as but not limited to Fluorescein (FITC), Alexa Fluor 350, 405, 488, 532, 546, 555, 568, 594, 647, 680, 700, 750, Pacific Blue, Coumarin, Pacific Green, Cy3, Texas Red, PE, PerCP-Cy5, PE-Cy7, Pacific Orange, or a fluorescent protein label such as R-PE or APC, or an expressed fluorescent protein such as CFP, EGFP, GFP or RFP. Such label may be used for in vitro detection or imaging of PS-positive dead or dying cells, cell debris, activated thrombocytes and/or extracellular vesicles, and in vitro staining and depleting assays of PS-positive dead or dying cells, cell debris, activated thrombocytes and/or extracellular vesicles as described further herein. However, also bioluminescent labels can be used in this respect, such as for example, bacterial luciferase (†uxAB), Photinus luciferase, ren/7/a luciferase or firefly luciferase.

When the label is a solid phase carrier, this term is understood as a (transporting) probe, substance or medium made of metal, polymere, sephadex, polystyrene, agarose, microspheres or other (polymeric) compounds. This solid phase carrier is capable of targeting or capable of binding to the additional molecule comprised by the PS binding agent as described elsewhere herein, such as streptavidin, avidin or neutravidin, thereby interacting with said additional molecule. Hence, these solid phase carriers are equally applicable as labels of the PS binding agent of the present invention. An overview of various solid phase carriers and their coupling to streptavidin has been described e.g. in El-Mandy et al. 2015, Microchim Acta (20151) 182:495-503, Peterman et al. 1988. J Immunol Methods 111:271-275 and Herrnanson et al. 1992, Immobilized Affinity Ligand Techniques. Academic, New York.

Preferably, said solid phase carrier is a bead. Beads applicable in this regard are preferably magnetic beads, such as dynabeads or nanobeads. Said magnetic beads may be coupled/coated to an antibody targeting or capable of binding to the PS binding agent of the present invention as described herein above. Preferably, said magnetic beads are coupled/coated to an antibody targeting or capable of binding to the additional molecule comprised in said PS binding agent as described elsewhere herein. In a most preferred embodiment said magnetic beads are coupled/coated to an antibody targeting or capable of binding to streptavidin, avidin or neutravidin. More preferred said magnetic beads can be directly or indirectly linked to said additional molecule such as streptavidin, avidin or neutravidin, as described e.g. in Zhang et al. 2007, Sci China Ser B-Chem, vol. 50, no.1, 127-134.

As set forth herein above, the label may further relate to microbubbles targeting or capable of binding to the PS binding agent of the present invention. Specifically, said microbubbles target the additional molecule such as streptavidin, avidin or neutravidin. Said microbubbles exist in many different sizes and material compositions as described e.g. in Lee et al. 2017, Biomed Eng Lett, 7(2): 59-69. Streptavidin microbubbles have been described in Matula et al. 2018, J Acoust Soc Am., 144(1): 41-52, Otani & Yamahara 2011, Molecular Imaging and Biology volume 13, pages 250-256 and Liou et al. 2015 (https://doi.org/10.1371/journal.pone.0125036) and are particularly useful.

As described elsewhere herein, labels such as solid phase carrier, fluorophores and microbubbles as defined herein above when comprised by the PS binding agent of the present invention or when added to the PS binding agent of the present invention may be particularly useful for in vitro depletion or enrichment of PS-positive dead or dying cells, cell debris, activated thrombocytes and/or extracellular vesicles from or in a sample comprising living cells.

The attachment of the label may be either direct or indirect via a linker, if the label is not a linker per se, or an additional molecule such as streptavidin, avidin or neutravidin. Thus, it is also comprised herein that the PS binding agent, when the label is not a linker, is linked to a label or connected/linked to a label via streptavidin, avidin or neutravidin. Said attachment of the label to the PS binding agent may also be covalently as defined elsewhere herein. Thus, it is also comprised herein that the binding agent is directly or indirectly labeled, preferably covalently.

The PS binding agent of the present invention may also be labeled as defined herein with a combination of any one of the labels mentioned elsewhere herein. For example, the PS binding agent may be labeled with a tag, preferably with a histidine tag, and with a biotin or with a fluorophore and with a biotin or with a tag, preferably with a histidine tag and a fluorophore. Therefore, the present invention comprises that the binding agent is labeled with any one of a tag, a fluorophore, a solid phase carrier, a microbubble, a linker or an active site probe, or a combination thereof.

The present invention may further comprise the PS binding agent as defined elsewhere herein, wherein said PS binding agent is capable of binding to PS on dead or dying cells, cell debris, activated thrombocytes and/or extracellular vesicle. In other words, the PS binding agent is capable of binding to PS-positive dead or dying cells, PS-positive cell debris, PS-positive activated thrombocytes and/or PS-positive extracellular vesicles. Thereby, dead or dying cells and/or cell debris can be efficiently removed from solutions such as a cell suspension comprising PS positive dead or dying cells and/or cell debris as described elsewhere herein. Alternatively, dead or dying cells and/or cell debris can be efficiently enriched in the solutions such as a cell suspension comprising PS positive dead or dying cells and/or cell debris as described elsewhere herein. Dead cells refer to cells which have already died through apoptosis or necrosis, i.e. programmed cell death, whereas dying cells in the context of the present invention are in the process of dying through apoptosis or necrosis. Thus, dead or dying cells as used herein may comprise apoptotic cells and/or necrotic cells. The cell debris from cell suspension may also refer to necrotic and/or apoptotic cell debris. Cell debris as used herein may refer to any cell fragments of said dead or dying cells as defined herein. Also activated thrombocytes become PS positive and therefore also the PS binding agent is capable of binding to PS on activated thrombocytes (Zwaal and Schroit (1997), Blood; 89:1121-32). The phosphatidylserine is part of the activated thrombocytes membrane and exposed on the activated living cell surface, where it can be bound by a PS binding agent via the PS-binding capability. As such, the method of detecting activated thrombocytes extends only to the detection of activated thrombocytes that have phosphatidylserine on the cell surface. Said extracellular vesicles, which are also PS-positive, are preferably associated/connected to living cells. “Extracellular vesicles” (EVs) is used in accordance with its well-known meaning to refer to membrane-contained vesicles that are released by cells of plants, prokaryotes and eukaryotes in an evolutionally conserved manner (Yanez-Mai et al., Journal of Extracellular Vesicles 2015, 4: 27066). Presently, three main subgroups of extracellular vesicles have been defined in the scientific literature: a) apoptotic bodies, b) cellular microparticles (also termed “microvesicles” or “ectosomes”), and c) exosomes (cf. Yanez-Mó et al., Journal of Extracellular Vesicles 2015, 4: 27066). Apoptotic bodies usually have a size ranging from about 1 to 5 μm diameter and are released when plasma membrane blebbing occurs during apoptosis, while the second group comprises vesicles of different sizes that pinch directly off the plasma membrane and have a size of about 100 to 1000 nm diameter. Exosomes have a size of about 30 to 100 nm diameter and are usually intraluminal vesicles (ILVs) contained in multi-vesicular bodies (MVBs), which are released to the extracellular environment upon fusion of MVBs with the plasma membrane (Colombo et al., Ann Rev Cell Dev Biol. 2014; 30:255-89). All three kinds of EVs can have phosphatidylserine exposed on their extravesicular surface and can in this case be bound by the PS binding agent of the present invention. In this regard, the PS-binding protein Annexin V was shown to bind to all three kinds of vesicles (see, e.g., Table 1 in Gyorgy, B. et al., CMLS 68, 2667-2688, 2011; Dignat-George, F. & Boulanger, C. M., Arteriosclerosis, thrombosis, and vascular biology 31, 27-33, 201 1, or Zwaal, R. F. & Schroit, A. J., Blood 89, 1121-1 132 (1997)). The phosphatidylserine is part of the EV membrane and exposed on the extravesicular surface, where it can be bound by a PS binding agent via the PS-binding capability. As such, the method of detecting extracellular vesicles extends only to the detection of EVs that have phosphatidylserine on the extravesicular surface. Preferably, the extracellular vesicle having phosphatidylserine (PS) on the extravesicular surface to be detected are apoptotic bodies and/or cellular microparticles. The detection of PS positive dead or dying cells, cell debris, activated thrombocytes and/or extracellular vesicles, preferably PS-positive cell-associated extracellular vesicles, which are often associated with diseases such as cancer, inflammation, infection and autoimmunity, was difficult so far and has now been overcome by applying the PS binding agent of the present invention. The dead or dying cells, cell debris, activated thrombocytes and/or extracellular vesicles having phosphatidylserine (PS) on the extravesicular surface to be detected can be present in vitro or ex vivo, e.g. in a cell culture dish or a tissue or organ explant obtained form a subject, and can be detected using the methods described elsewhere herein.

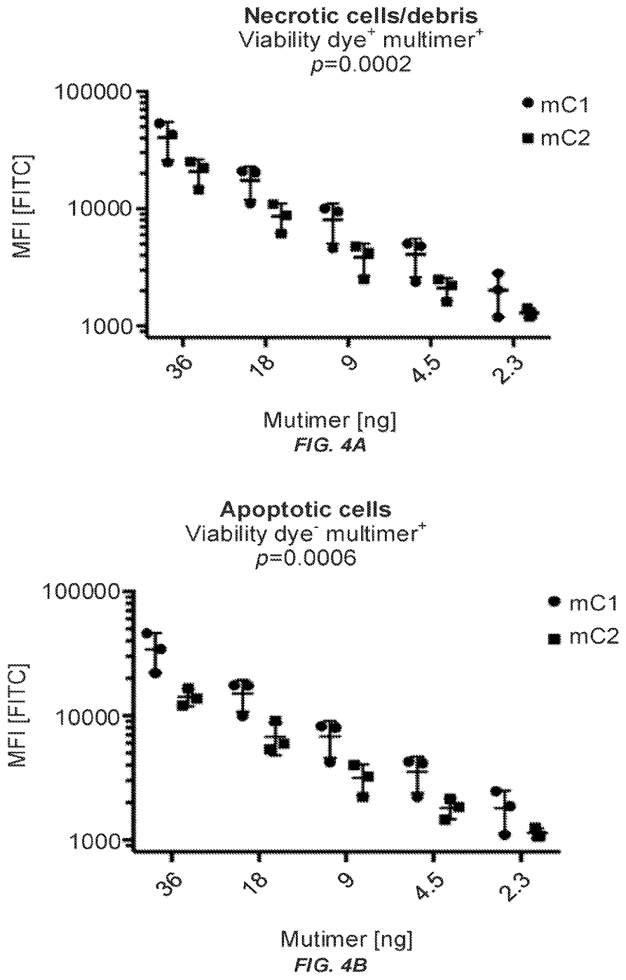

In more detail, said PS binding agent is capable of binding to PS on (apoptotic and/or necrotic) dead or dying cells, cell debris, activated thrombocytes and/or extracellular vesicles, preferably cell-associated extracellular vesicles, via its one or more (e.g., at least two) C-domains. As demonstrated by the present invention, said binding of C1-multimers under physiological conditions bind dead cells more potently when compared to C2-multimers (see FIG. 4). In particular, when the PS binding agent has one or more (e.g., at least two) C-domains, each C-domain (either the C1- or the C2-domain or a fragment thereof) binds to a different PS located on the same dead or dying cell, cell debris, activated thrombocyte and/or extracellular vesicle (preferably cell-associated extracellular vesicle). For example if the binding agent has four C1-domains, each C1-domain binds to a different PS on the same dead or dying cell, cell debris, activated thrombocyte and/or extracellular vesicle (preferably cell-associated extracellular vesicle).

### Composition

The present invention also relates to a composition comprising at least one PS binding agent as defined elsewhere herein. Said composition refers to a diagnostic composition. When the at least one PS binding agent is labeled as defined herein for diagnostic purposes comprising in vitro detection, said composition comprising said at least one PS binding agent refers to a diagnostic composition. Moreover, the present invention relates to the use of a PS binding agent as disclosed herein above for the preparation of a diagnostic composition.

The term “diagnostic composition” when used herein refers to a composition comprising at least one of the PS binding agent of the present invention, which can be applied for detection and diagnosis in vitro.

The present invention may also encompass the composition as defined herein, further comprising a diagnostically acceptable excipient. Hence, the diagnostic composition of the present invention further comprises a diagnostically acceptable carrier, diluent or excipient. Said terms can be used interchangeably. Said diagnostically acceptable excipient includes also any excipient that does not itself elicit an adverse reaction, which would be harmful when used in vitro. Suitable excipients are typically large, slowly metabolized macromolecules such as proteins, polysaccharides, polylactic acids, polyglycolic acids, polymeric amino acids, amino acid copolymers and lipid aggregates such as, e.g. oil droplets or liposomes. The carrier used in combination with the PS binding agent of the present invention may be water-based and forms an aqueous solution. An oil-based carrier solution containing the compound of the present invention is an alternative to the aqueous carrier solution. Either aqueous or oil-based solutions further contain thickening agents to provide the composition with the viscosity of a liniment, cream, ointment, gel, or the like. Suitable thickening agents are well known to those skilled in the art. Alternative embodiments of the present invention can also use a solid carrier containing the diagnostic compound for use in diagnosis as disclosed elsewhere herein. This enables the alternative embodiment to be applied via a stick applicator, patch, or suppository. The solid carrier further contains thickening agents to provide the composition with the consistency of wax or paraffin.

Diagnostically acceptable excipients according to the present invention include, by the way of illustration and not limitation, diluent, disintegrants, binding agents, adhesives, wetting agents, polymers, lubricants, gliands, substances added to mask or counteract a disagreeable texture, taste or odor, flavors, dyes, fragrances, and substances added to improve appearance of the composition. Acceptable excipients include lactose, sucrose, starch powder, maize starch or derivatives thereof, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, saline, dextrose, mannitol, lactose, lecithin, albumin, sodium glutamate, cysteine hydrochloride, and the like. Examples of suitable excipients for soft gelatin capsules include vegetable oils, waxes, fats, semisolid and liquid polyols. Suitable excipients for the preparation of solutions and syrups include, without limitation, water, polyols, sucrose, invert sugar and glucose. Suitable excipients for injectable solutions include, without limitation, water, alcohols, polyols, glycerol, and vegetable oils. The diagnostic compositions can additionally include preservatives, solubilizers, stabilizers, wetting agents, emulsifiers, sweeteners, colorants, flavorings, buffers, coating agents, or antioxidants. Suitable pharmaceutical and diagnostic carriers are described in Remington's Pharmaceutical Sciences, Mack Publishing Company, a standard reference text in this field.

Further, the excipients of the diagnostic composition may also refer to diluents such as, e.g. water, saline, glycerol, ethanol, bacteriostatic water for injection (BWFI), Ringer's solution, dextrose solution, or aqueous solutions of salts and/or buffers etc. Furthermore, substances necessary for formulation purposes may be comprised in said compositions as acceptable excipients such as emulsifying agents, stabilizing agent, surfactants and/or pH buffering substances known to a person skilled in the art.

Said stabilizing agent/stabilizer may act as a tonicity modifier. The term “stabilizing agent” refers to an agent that improves or otherwise enhances stability of the formulation, in particular of the at least one PS binding agent. A stabilizing agent which is a tonicity modifier may be a non-reducing sugar, a sugar alcohol or a combination thereof. The tonicity modifiers of the compositions of the present invention ensure that the tonicity, i.e., osmolarity, of the solution is essentially the same as normal physiological fluids and may thus prevent post-administration swelling or rapid absorption of the composition because of differential ion concentrations between the composition and physiological fluids. Preferably, the stabilizing agent/tonicity modifier is one or more of non-reducing sugars, such as sucrose or trehalose or one or more of sugar alcohols, such as mannitol or sorbitol, also combinations of non-reducing sugars and sugar alcohols are preferred.

In compositions of the present invention, the addition of surfactants can be useful to reduce protein degradation during storage. The polysorbates 20 and 80 (Tween 20 and Tween 80) are well established excipients for this purpose. Persons having ordinary skill in the art will understand that the combining of the various components to be included in the formulation can be done in any appropriate order. It is also to be understood by one of ordinary skill in the art that some of these chemicals can be incompatible in certain combinations, and accordingly, are easily substituted with different chemicals that have similar properties but are compatible in the relevant mixture.

The term “buffering agent” as used herein, includes those agents that maintain the pH in a desired range. A buffer is an aqueous solution consisting of a mixture of a weak acid and its conjugate base or a weak base and its conjugated acid. It has the property that the pH of the solution changes very little when a small amount of a strong acid or base is added. Buffer solutions are used as a means of keeping pH at a nearly constant value in a wide variety of chemical applications. In general, a buffer when applied in the formulation of the invention preferably stabilizes the at least one PS binding agent.

Said composition of the present invention may be a liquid, preferably aqueous, composition. Further comprised herein is a dried or frozen form of the composition as defined herein. Thus, said composition may be stored directly in liquid form for later use, stored in a frozen state and thawed prior to use, or prepared in dried form, such as a lyophilized, air-dried, or spray-dried form, for later reconstitution into a liquid form or other form prior to use. Thus, it is envisaged that a composition described herein may be stored by any method known to one of skill in the art. Non-limiting examples include cooling, freezing, lyophilizing, and spray drying the formulation, wherein storage by cooling is preferred. Mostly preferred is long-term storage at −80° C., storage for several months at −20° C. and short-term storage at 4° C. as liquid form in stabilizing buffer. In this context, the term “long-term storage” refers to a storage of at least about 6 month, such as about 6, 7, 8, 9, 10, 15, 20, 24, 36 month or more, whereas the term “short-term storage” refers to a storage of at most about 2 month, such as about 2 month, 1 month, 3 weeks, 2 weeks, 1 week or even only several days.

The fact that the composition of the present invention “comprising at least one PS binding agent” as used herein means that said composition comprising one, two, three, four, five or more binding PS binding agents according to the present invention.

In the context of the present invention, the composition as defined elsewhere herein preferably comprises a solution being calcium-independent. The term “buffer” may be used interchangeably with the term “solution”. The term “calcium-independent” according to the method of the present invention means that the solution used in the composition of the present invention does not need to comprise calcium. However, if the solution used comprises calcium, such solution would still be suitable for the composition of the present invention. Thus, the addition of calcium does not play any role in the applied solution of the composition.

In a preferred embodiment said solution being calcium-independent comprises arginine, leucine, glutamic acid, glycerol, NaCl, and/or HEPES. Preferably, such solution has a pH of 7.4. In an even more preferred embodiment said solution being calcium-independent comprises 18 mM arginine, 3.5 mM leucine, 5.7 mM glutamic acid, 8% glycerol, 500 mM NaCl, 25 mM HEPES having a pH of 7.4. Alternatively, in another preferred embodiment said solution being calcium-independent comprises HEPES, glycerol, arginine, glutamic acid, NaCl. Preferably, also such solution has a pH of 7.4. In another even more preferred embodiment said solution being calcium-independent comprises 200 mM arginine, 200 mM glutamic acid, 2% glycerol, 150 mM NaCl, 25 mM HEPES having a pH of 7.4.

### Kits

The present invention also relates to a kit comprising the PS binding agent as defined herein or the composition as defined herein by the present invention. Thus, when a kit comprises the PS binding agent per se, said binding agent may be provided in a vial or a container. Further it may be associated with a notice in the form prescribed by a governmental agency regulating the manufacture, use or sale of pharmaceuticals or biological products, reflecting approval by the agency of the manufacture, use or sale of the product for human administration or diagnostics. Said kit may comprise the PS binding agent, preferably in a vial or container, in dried form, such as a lyophilized, air-dried, or spray-dried form (in form of a powder), for later reconstitution into a liquid form or other form prior to use. Further, said kit may also comprise the PS binding agent, preferably in a vial or container, in a frozen state, being thawed prior to use. According to the present invention, the kit comprising the PS binding agent may further comprise a diagnostically acceptable excipient, and/or a solution being calcium-independent as defined elsewhere herein. In some embodiments, said excipient, and/or said solution being calcium-independent as defined elsewhere herein may also be comprised in one or more containers or vials in said kit, meaning said kit additionally comprising either one vial or container comprising said excipient, and/or said solution as a mixture or said kit additionally comprising for each component such as the excipient, and/or the solution separate vials or containers. Said kit as defined herein may be suitable for staining PS-positive dead or dying cells, cell debris, activated thrombocytes and/or extracellular vesicles or for depleting PS-positive dead or dying cells, cell debris, activated thrombocytes and/or extracellular vesicles as defined elsewhere herein.

When a kit comprises the composition as defined elsewhere herein, said composition may be a diagnostic composition as defined herein. Preferably, said kit comprising the diagnostic composition as defined herein is suitable for staining PS-positive dead or dying cells, cell debris, activated thrombocytes and/or extracellular vesicles or for depleting PS-positive dead or dying cells, cell debris, activated thrombocytes and/or extracellular vesicles.

According to the present invention, the compositions as defined herein are preferably provided in one or more containers or vials in said kit (diagnostic pack), which may also be associated with a notice in the form prescribed by a governmental agency regulating the manufacture, use or sale of biological products, reflecting approval by the agency of the manufacture, use or sale of the product for diagnostics.

Thus, the present invention may comprise a kit comprising one vial or container comprising the composition as defined herein comprising at least one PS binding agent, wherein the composition additionally comprises the acceptable excipient, and/or the solution as defined herein. Also comprised by the present invention, is a kit comprising one or more vials or containers each comprising the composition as defined herein comprising at least one PS binding agent of the present invention, wherein each composition in said vial or container additionally comprises the acceptable excipient, and/or the solution as defined herein.

According to the present invention, when the kit as defined above may be suitable for depleting PS-positive dead or dying cells, cell debris, activated thrombocytes and/or extracellular vesicles, said kit may further comprise a compound capable of targeting said PS binding agent. Said compound may refer to an antibody, or beads coupled/coated to an antibody, preferably dynabeads coupled/coated to an antibody. In some embodiments, said compound may also be comprised in the one or more containers or vials of the kit as defined above or in additional one or more vials or containers of said kit, preferably further comprising in said one or more vials or containers any excipient suitable for said compound to be mixed with/contacted with. In a preferred embodiment, said compound is an anti-Streptavidin or anti-avidin or anti-neutravidin antibody, or a bead (dynabead) coupled/coated to an anti-Streptavidin or anti-avidin or anti-neutravidin antibody.

Additionally, the present invention also provides another kit which enables the skilled person to produce yourself the multimeric PS binding agent as defined throughout the present invention. Thus, said the present invention also relates to a kit comprising at least two C-domains in a monomeric form of a MFG-E8 protein and an additional molecule capable of linking said C-domains to each other. The at least two C-domains of a MFG-E8 protein as used herein may refer for some instances as defined below to the at least two C-domains already defined elsewhere herein which are comprised in the PS binding agent of the present invention. One of the differences here is that said at least two C-domains used in said kit are not already directly or indirectly linked, preferably via streptavidin, neutravidin or avidin, as it is for the at least two C-domains in the PS binding agent as defined elsewhere herein, which provides a multimer. The respective C-domains of said kit are present as monomers, not being directly or indirectly linked as described herein for said C-domains of said PS binding agent. Thus, the C-domains of said kit are monomeric. The C-domains of said kit will be linked (either directly or indirectly) to each other in the future when applying said kit, which additionally comprises said additional molecule, which is capable of linking said C-domains to each other under conditions allowing the formation of a PS binding agent comprising at least two C-domains defined elsewhere herein. Thus, the end product when having applied said kit is the multimeric PS binding agent of the present invention which comprises said at least two C-domains of a MFG-E8 protein. Said kit can therefore be seen as a kit suitable for do-it-yourself tinkering.

The “additional molecule capable of linking said C-domains to each other” in the context of the present invention refers to any molecule which has the capability of bringing the at least two C-domains of said kit together in any way. In particular, said C-domains will be indirectly linked in the future by the additional molecule comprised in said kit, preferably covalently linked as defined elsewhere herein. Said additional molecule may not be able to directly link said C-domains to another. When the molecule is capable of linking said C-domains indirectly to each other, it can also be used herein that said additional molecule multimerizes said C-domains. Thus, it is also comprised by the present invention that a kit is encompassed comprising at least two C-domains of a MFG-E8 protein as defined herein and an additional molecule capable of multimerizing said C-domains. The term “multimerize”/“multimerizing” refers to making a multimer of said at least two monomeric C-domains, thus brining said monomers together, linking them to each other indirectly, thereby producing/forming said multimer of the present invention, namely the PS binding agent. Preferably, said additional molecule is streptavidin or avidin, or neutravidin, more preferably streptavidin. The present invention may thus comprise a kit as defined herein, wherein said additional molecule is streptavidin or avidin, or neutravidin. When the additional molecule is streptavidin, preferably site-specific modification with a biotin-containing peptide as defined herein allows multimerization of said at least two C-domains provided in said kit. Preferably, said additional molecule further comprises a label. Said label is preferably, a tag, a fluorophore, a solid carrier, a microbubble, a linker or an active site probe as defined elsewhere herein. Preferably, said solid phase carrier is a bead that may be directly or indirectly linked to said additional molecule or coupled/coated via an antibody targeting said additional molecule. Equally preferred in this context is a microbubble comprised by said additional molecule.

According to the present invention, the monomeric one or more (e.g., at least two) C-domains as defined herein are preferably provided in one or more containers or vials in said kit (either as a mixture comprising all of the one or more (e.g., at least two) C-domains or each C-domains separately in one container or vial). Said additional molecule may also be comprised in the same container(s) or vial(s) comprising the C-domains or it may be separately provided in an additional container or vial. Said kit may also be associated with a notice in the form prescribed by a governmental agency regulating the manufacture, use or sale of said products, reflecting approval by the agency of the manufacture, use or sale of the product for human administration or diagnostics.

Said kit may further comprise any excipient as defined herein, which may be comprised in said one or more containers or vials comprising said one or more (e.g., at least two) C-domains and/or comprising said additional molecule.

The present invention may also comprise said kit, wherein said C-domains are any one of a C1-domain of MFG-E8 protein, a C2-domain of MFG-E8 protein or a fragment thereof, preferably a C1-domain of MFG-E8 protein. Also comprised is a kit as defined herein, wherein said C1-domain comprises an amino acid sequence having at least about 60% sequence identity with an amino acid sequence of SEQ ID NO: 1. Alternatively or simultaneously, the present invention may also encompass said kit as defined herein, wherein said C2-domain comprises an amino acid sequence having at least about 60% sequence identity with an amino acid sequence of SEQ ID NO: 2. In a preferred embodiment, said C1-domain has the amino acid sequence of SEQ ID NO: 1 and/or said C2-domain has the amino acid sequence of SEQ ID NO: 2. For these specific embodiments, each disclosure or each definition already made throughout the specification regarding the one or more (e.g., at least two) C-domains of the PS binding agent of the present invention can be considered.

In order that said one or more (e.g., at least two) C-domains comprised in said kit can be modified enzymatically, said kit may further comprise an enzyme for enzymatic modification of said C-domains. Thus, the present invention also refers to a kit as defined elsewhere herein, further comprising an enzyme for enzymatic modification of said C-domains. Any enzyme capable of modifying said one or more (e.g., at least two) C-domains enzymatically may be comprised herein. Enzymatic modification is known to a person skilled in the art. Said enzyme may again be either provided in one or more containers or vials of said kit already comprising said one or more (e.g., at least two) C-domains or it may be separately provided in an additional container or vial of said kit, preferably also comprising any suitable excipient known to a person skilled in the art which is applied for storage or usage of said enzyme in said kit.

When said kit further comprises an enzyme for enzymatic modification of said C-domains as defined herein, said C-domains also comprise a recognition sequence for said enzyme. Thus, the present invention may comprise a kit as defined elsewhere herein, wherein if said kit further comprises an enzyme for enzymatic modification of said C-domains, said C-domains comprise a recognition sequence for said enzyme for enzymatic modification of said C-domains. Said recognition sequence as comprised in any one of said one or more (e.g., at least two) C-domains or in all of said one or more (e.g., at least two) C-domains is necessary that said enzyme, which will modify said C-domains of said kit enzymatically, will recognize said target for enzymatic modification. The disclosure for said recognition sequence as defined elsewhere herein may be applicable here.

Preferably, the invention comprises a kit as defined elsewhere herein, wherein said enzyme for enzymatic modification of said C-domains is a biotin ligase or a sortase A, even more preferably sortase A. If the enzyme for enzymatic modification of said C-domains comprised in said kit as defined herein is a biotin ligase, said enzyme of said kit is BirA. Again, the disclosure for said particular enzymes as defined elsewhere herein may be applicable here.

In addition, if the kit of the present invention comprises as an enzyme for enzymatic modification of said C-domains a biotin ligase, preferably BirA, or a sortase A, said kit as defined elsewhere herein further comprises biotin. Since a biotin ligase, in particular BirA, or sortase A catalyzes the reaction of biotinylation of a protein of interest, in the context of the present invention the one or more (e.g., at least two) C-domains of said kit, said kit then further comprises biotin in order to provide the biotin-containing peptide for said biotinylation process. When having sortase A or any biotin ligase, in particular BirA, and additionally also biotin comprised in said kit, the one or more (e.g., at least two) C-domains, which are monomeric in said kit, can be become biotinylated. In a most preferred embodiment, if the kit of the present invention comprises as an enzyme for enzymatic modification of said C-domains sortase A, said kit as defined elsewhere herein, further comprises biotin. Said biotin molecule may again be either provided in one or more containers or vials of said kit already comprising said one or more (e.g., at least two) C-domains and said biotin ligase, in particular BirA, or sortase A or it may be separately provided in an additional container or vial of said kit already comprising said biotin ligase, in particular BirA, or sortase A, or it may be separately provided alone in an additional container or vial of said kit, preferably also comprising any suitable excipient known to a person skilled in the art which is applied for storage or usage of said biotin in said kit. In this embodiment said one or more (e.g., at least two) C-domains are not biotinylated yet. Only by applying said kit which comprises each component for said biotinylation process (sortase A or biotin ligase —BirA-, biotin and said C-domains), said one or more (e.g., at least two) C-domains will then be biotinylated.

The present invention preferably refers to a kit as defined elsewhere herein, wherein said C-domains are already biotinylated as defined herein. Again, this can be achieved by applying said kit comprising each component for said biotinylation process such as the particular enzymes as defined herein, a biotin molecule and said one or more (e.g., at least two) C-domains or already biotinylated C-domains as defined herein may be comprised in said kit. Accordingly, the present invention may therefore comprise a kit as defined elsewhere herein, wherein said C-domains are biotinylated with sortase A or a biotin ligase, preferably wherein said biotin ligase is Bir A, even more preferably wherein said C-domains are biotinylated with sortase A.

### In Vitro and In Vivo Diagnostic Applications

Also provided by the present invention is an in vitro method of staining PS-positive dead or dying cells, cell debris, activated thrombocytes and/or extracellular vesicles in a sample from a subject, the method comprising a) contacting said sample with a PS binding agent as defined elsewhere herein comprising a fluorophore, and optionally b) detecting said PS binding agent using an imaging method.

Hence, the present invention also refers to the use of the PS binding agent as defined elsewhere herein, the composition as defined elsewhere herein, or the kit defined elsewhere herein for staining PS-positive dead or dying cells, cell debris, activated thrombocytes and/or extracellular vesicles.

The term “stain or staining” as used herein means that the PS binding agents of the present invention can be used to highlight or mark PS-positive dead or dying cells, cell debris, activated thrombocytes and/or extracellular vesicles in a sample. Hence, the PS binding agent of the present invention in accordance with said method is used as staining reagent directly reacting with said PS-positive dead or dying cells, cell debris, activated thrombocytes and/or extracellular vesicles in said sample from a subject, thereby “labeling” said PS-positive dead or dying cells, cell debris, activated thrombocytes and/or extracellular vesicles. The staining step can be performed by contacting the sample obtained from a subject with the PS binding agent of the present invention further comprising a fluorophore using condition known to those skilled in the art. Hence, the method of staining PS-positive dead or dying cells, cell debris, activated thrombocytes and/or extracellular vesicles in a sample from a subject as described herein comprises the use of PS binding agents comprising a fluorophore as defined elsewhere herein. In this respect the diagnostic composition or the kit comprising a PS binding agent as defined elsewhere herein are applicable for said method of staining PS-positive dead or dying cells, cell debris, activated thrombocytes and/or extracellular vesicles in a sample.

Said method may further comprise detecting said labeled PS binding agent bound to said PS-positive dead or dying cells, cell debris, activated thrombocytes and/or extracellular vesicles using an optical imaging method. Optical imaging techniques applicable for the detection of PS positive dead or dying cells, cell debris, activated thrombocytes and/or extracellular vesicles stained with the PS binding agent of the present invention in vitro comprise but are not limited to flow cytometry, such as imaging flow cytometry, confocal microscopy, two-photon microscopy, laser-scanning microscopy, transmission electron microscopy, and coherent anti-Stokes-Raman scattering (CARS) microscopy. Accordingly, said in vitro method of staining PS-positive dead or dying cells, cell debris, activated thrombocytes and/or extracellular vesicles in a sample from a subject can be used as in vitro diagnostic method for the diagnosis of diseases or disorders associates with the exposure of PS-positive dead or dying cells, cell debris, activated thrombocytes and/or extracellular vesicles as described elsewhere herein. Non-limiting examples samples applicable in this respect are blood samples, tissues samples, body fluid samples, skin samples or any other samples known to those skilled in the art for use in in vitro diagnosis. The skilled person is further aware of methods for obtaining samples from a subject, such as blood sampling, smears or biopsy.

The term “subject” when used herein includes mammalian and non-mammalian subjects. Preferably the subject of the present invention is a mammal, including human, domestic and farm animals, non-human primates, and any other animal that has mammary tissue. In some embodiment the mammal is a mouse. In some embodiment the mammal is a rat. In some embodiment the mammal is a guinea pig. In some embodiment the mammal is a rabbit. In some embodiment the mammal is a cat. In some embodiment the mammal is a dog. In some embodiment the mammal is a monkey. In some embodiment the mammal is a horse. In a most preferred embodiment the mammal of the present invention is a human. A subject also includes human and veterinary patients.

As set forth elsewhere herein, PS positive dead or dying cells, cell debris, activated thrombocytes and/or extracellular vesicles can often be found in patients suffering from a diseases such as cancer, inflammation, infection or autoimmune disease as defined elsewhere herein. In fact, many types of cancer, inflammatory diseases, infections and autoimmune diseases are known to be associated with the presence of PS positive dead or dying cells, cell debris, activated thrombocytes and/or extracellular vesicles, preferably PS-positive cell-associated extracellular vesicles. Since dead or dying cells exposure PS extracellular on their membrane and are excessively produced in the course of said diseases, PS binding agents as defined in the present invention are well suitable for detect said cells in a sample from a subject, thereby allowing the diagnosis of said diseases. Hence, the present invention also refers to the use of the PS binding agents, the composition or the kit as defined herein for the staining PS-positive dead or dying cells, cell debris, activated thrombocytes and/or extracellular vesicles or for depleting PS-positive dead or dying cells, cell debris, activated thrombocytes and/or extracellular vesicles, thereby diagnosing diseases associated with the presence of PS positive dead or dying cells, cell debris, activated thrombocytes and/or extracellular vesicles.

In the context of diagnostic applications of the PS binding agents of the present invention, it is particularly envisaged that said PS binding agents is used in form of the diagnostic composition described elsewhere herein. The term “diagnosing” or “diagnosis” when used herein means determining or detecting if a subject suffers from a disease or disorder. Preferably, said diagnosis is diagnosis of a disease or disorder selected from the group consisting of cancer, inflammation, infection and autoimmune disease in a subject as defined elsewhere herein. However, where reference is made to “diagnosis” of such a disease, this should be taken to include both diagnosis of the disease itself, as well as susceptibility to the disease. Accordingly, the in vitro methods of diagnosis disclosed herein may also be employed as methods of providing indications useful in the diagnosis of such a disease. Typically, the disease or disorder as described herein is associated with the presence of PS positive dead or dying cells, cell debris, activated thrombocytes and/or extracellular vesicles, preferably PS-positive cell-associated extracellular vesicles. Thus, said disease or disorder is associated with an increased amount of PS positive dead or dying cells, cell debris, activated thrombocytes and/or extracellular vesicles, preferably PS-positive cell-associated extracellular vesicles in said subject when compared to a subject not suffering from said disease or disorder. Thus, the presence of said PS positive dead or dying cells, cell debris, activated thrombocytes and/or extracellular vesicles, is above the normal level of PS positive dead or dying cells, cell debris, activated thrombocytes and/or extracellular vesicles in a particular tissue in said subject. Accordingly, said PS positive dead or dying cells, cell debris, activated thrombocytes and/or extracellular vesicles may be enriched in said particular tissue in said subject.

The term “disease” or “disorder” as used herein, refers to any physical state of a subject connected with incorrectly functioning organ, part, structure, or system of the body resulting from the effect of genetic or developmental errors, infection, poisons, nutritional deficiency or imbalance, toxicity, or unfavorable environmental factors, illness, sickness, or ailment. The term “disease” or “disorder” further includes any impairment of the normal physical state of the subject or one of its parts that interrupts or modifies the performance of vital functions that are typically manifested by distinguishing signs and symptoms.

The term “cancer” as used herein refers to diseases caused by uncontrolled cell division and the ability of cells to metastasize, or to establish new growth in additional sites. The terms “malignant”, “malignancy”, “neoplasm”, “tumor,” “cancer” and variations thereof refer to cancerous cells or groups of cancerous cells. Specific types of cancer include, but are not limited to, skin cancers (e.g., melanoma), connective tissue cancers (e.g., sarcomas), adipose cancers, breast cancers, head and neck cancers, lung cancers (e.g., mesothelioma), stomach cancers, pancreatic cancers, ovarian cancers, cervical cancers, uterine cancers, anogenital cancers (e.g., testicular cancer), kidney cancers, bladder cancers, colon cancers, prostate cancers, central nervous system (CNS) cancers, retinal cancer, blood, neuroblastomas, multiple myeloma, and lymphoid cancers (e.g., Hodgkin's and non-Hodgkin's lymophomas).

The term “inflammation” or “inflammatory disease” when used herein refers to a part of complex biological response of vascular tissues to harmful stimuli, such as pathogens, damaged cells, or irritants. In this context, inflammation is a protective response involving host cells, blood vessels, and proteins and other mediators that is intended to eliminate the initial cause of cell injury, as well as the necrotic cells and tissues resulting from the original insult, and to initiate the process of repair. Within the scope of the present invention, the inflammation described herein can be generally classified as either acute or chronic. The term “Inflammatory diseases” or “inflammatory disorder” when used herein refers to any physical state of the subject of the present invention which is related to a disease or a physical reaction connected with the occurrence of inflammation in said subject.

The term “infection” refers to the invasion of an organism's body tissues by disease-causing agents, their multiplication, and the reaction of host tissues to the infectious agents and the toxins they produce. An infectious disease, also known as a transmissible disease or communicable disease, is an illness resulting from an infection. Comprised in this respect are bacterial and viral infections, and infection with fungi, prions, parasites, and anthropodes. Preferably, said infection a viral disease, more preferred an infection with SARS-CoV-2.

The term “autoimmune disease” can be used interchangeably with the term “autoimmune disorder” and refers to a condition in a subject characterized by cellular, tissue and/or organ injury caused by an immunologic reaction of the subject to its own cells, tissues and/or organs. “Autoimmune disease” is typically associated with the production of antibodies and/or T cells directed against a self-antigen thereby causing the pathology of the disease. Autoimmune diseases in which autoantibodies are involved include, for example, bullous pemphigoid, Graves' disease, some forms of diabetes mellitus, myasthenia gravis, systemic lupus erythematosus, pernicious anaemia, autoimmune hemolytic anaemia, glomerulonephritis, autoimmune thrombocytopenic purpura. Self-reactive T cells are thought to be involved in rheumatoid arthritis and insulin-dependent diabetes mellitus. Other autoimmune disease are Sjogren's disease, type I diabetes, insulin dependent diabetes mellitus, scleroderma, polymyositis, chronic active hepatitis, mixed connective tissue disease, primary biliary cirrhosis, pernicious anemia, autoimmune hemolytic anemia, autoimmune thyroiditis, idiopathic Addison's disease, vitiligo, gluten-sensitive enteropathy, Grave's disease, myasthenia gravis, neutropenia, idiopathic thrombocytopenia purpura, pemphigus vulgaris, autoimmune infertility, Goodpasture's disease, bullous pemphigoid, discoid lupus, systemic lupus erythematosus, dense deposit disease, Hashimoto's disease, fibromyalagia, arthritis selected from rheumatoid arthritis, gouty arthritis, and juvenile rheumatoid arthritis, an autoimmune disease of blood vessels selected from necrotizing angitis, and granulomatous angitis, or an autoimmune disease of kidney selected from nephritis, glomerulonephritis, antiphospholipd syndrome, and systemic lupus erythematosus.

In a preferred embodiment of the methods and uses of the present invention, the disease associated with the presence of PS positive dead or dying cells, cell debris, activated thrombocytes and/or extracellular vesicles is a disease associated with a defect in phagocytosis of said dead or dying cells, cell debris, activated thrombocytes and/or extracellular vesicles. A “defect in phagocytosis” refers to defects in the uptake of dead or dying cells by phagocytes, as well as to defects in the degradation of phagocytosed cells within the phagocytes, for example due to insufficient amounts of Dnase present in macrophages. Such defects in phagocytosis can lead to an accumulation of dead cells and cell debris, which in turn can, for example, induce an autoimmune response in the subject concerned. Accordingly, a disease associated with a defect in phagocytosis is a disease caused or associated with one (or several) of these aspects. Preferably, the disease associated with a defect in phagocytosis is an autoimmune disease, more preferably a disease selected from lupus erythematosus and rheumatoid arthritis.

Hence, the in vitro method of staining PS-positive dead or dying cells, cell debris, activated thrombocytes and/or extracellular vesicles in a sample from a subject as defined herein allows for the diagnosis of a diseases associated with the presence of PS positive dead or dying cells, cell debris, activated thrombocytes and/or extracellular vesicles as defined herein in a subject when contacting a sample from said subject with a PS binding agent as defined herein and detecting said PS binding agent. In this respect said method may comprise a) contacting said sample with a PS binding agent as defined elsewhere herein comprising a fluorophore, and b) detecting said PS binding agent using an imaging method, thereby collecting imaging data. The signal in the imaging data received during the detection step can be considered as the reflected signal received from the fluorophore of the PS binding agent used to contact said sample. The term “imaging” when used herein refers to the optical visualization of PS positive dead or dying cells, cell debris, activated thrombocytes and/or extracellular vesicles in a sample from a subject under in vitro conditions, using the PS binding agent of the present invention linked to a label suitable label for in vitro imaging techniques described elsewhere herein. The method may further comprise c) comparing the imaging data received in step b) to reference imaging data. When comparing the collected imaging data from the subject to reference imaging data, the method equally comprises comparing the signal received from the fluorophore of the PS binding agent to a detected reference signal. Thus, in particular, the methods and uses of the present invention allow the comparison and quantification of said signals.

The reference imaging data or the detected reference signal typically derives from a sample obtained from a reference subject, which is a healthy subject. The term “healthy subject” when used herein refers to a subject not suffering from a disease associated with the presence of PS positive dead or dying cells, cell debris, activated thrombocytes and/or extracellular vesicles in said patient. In this regard, the collected imaging data for the subject to be diagnoses indicate the level of PS positive dead or dying cells, cell debris, activated thrombocytes and/or extracellular vesicles in the sample from said subject, and the reference imaging data indicate the level of PS positive dead or dying cells, cell debris, activated thrombocytes and/or extracellular vesicles in the sample from said reference subject. Accordingly, an increased signal in the imaging data in the sample from the subject to be diagnosed as compared to reference imaging data indicates the presence of a disease associated with the presence of PS positive dead or dying cells, cell debris, activated thrombocytes and/or extracellular vesicles in said subject. Alternatively, no difference in the signal in the imaging data in the sample from the subject to be diagnosed as compared to reference imaging data indicates no presence of a disease associated with the presence of PS positive dead or dying cells, cell debris, activated thrombocytes and/or extracellular vesicles in said subject.

Further, the in vitro staining of PS-positive dead or dying cells, cell debris, activated thrombocytes and/or extracellular vesicles in a sample from a subject as described herein above can be used for monitoring or evaluating the progression of a disease associated with the presence of PS positive dead or dying cells, cell debris, activated thrombocytes and/or extracellular vesicles in a subject as defined elsewhere herein. Said in vitro method may comprise a) contacting said sample with a PS binding agent as defined elsewhere herein comprising a fluorophore, and b) detecting said PS binding agent using an imaging method, thereby collecting imaging data. Said method may further comprise (c) comparing the imaging data received in step (b) to reference imaging data obtained for a sample from said patient at an earlier date, wherein the result of the comparison of (c) provides an evaluation of the progression of said disease associated with the presence of PS positive dead or dying cells, cell debris, activated thrombocytes and/or extracellular vesicles in said patient. In this regards, the term “monitoring or evaluating the progression” refers to any procedure or method used for the described in vitro application to assess whether or not a patient suffering from a disease associated with the presence of PS positive dead or dying cells, cell debris, activated thrombocytes and/or extracellular vesicles is responsive to treatment with a therapeutic agent, i.e. for determining the effectiveness of a therapeutic treatment. In particular, a method of monitoring or evaluating the progression of a disease associated with the presence of PS positive dead or dying cells, cell debris, activated thrombocytes and/or extracellular vesicles relates to monitoring or evaluating the level of PS positive dead or dying cells, cell debris, activated thrombocytes and/or extracellular vesicles in a sample from a subject prior, during and after therapy with a therapeutic agent. The term “therapeutic agent” as used herein refers to any compounds suitable to treat said disease associates with PS positive dead or dying cells, cell debris, activated thrombocytes and/or extracellular vesicles. Accordingly, the method of monitoring as described herein particularly refers to monitoring the therapeutic efficacy of a drug used in the treatment of a disease associates with PS positive dead or dying cells, cell debris, activated thrombocytes and/or extracellular vesicles. Hence, conclusions can be drawn during and/or after the treatment of a subject with the drug/medicament/therapeutic agent as to whether said medicament may improve symptoms of said disease when comparing to the physical conditions before start of treatment. Moreover, such monitoring or evaluation may help an attending physician to obtain the appropriate information to set the appropriate therapy conditions for the treatment of said disease associates with PS positive dead or dying cells, cell debris, activated thrombocytes and/or extracellular vesicles inflammatory disease. A significantly increased signal in the imaging data in the sample from the subject to be monitored as compared to reference imaging data obtained from said patient at an earlier date indicates a progression of the disease associated with the presence of PS positive dead or dying cells, cell debris, activated thrombocytes and/or extracellular vesicles in said patient, whereas no change or a decreased signal in the imaging data in the sample from the subject to be monitored as compared to reference imaging data obtained from said patient at an earlier date indicates no progression or a regression of the disease associated with the presence of PS positive dead or dying cells, cell debris, activated thrombocytes and/or extracellular vesicles in said patient.

Further, as shown by the inventors of the present invention, the PS binding agents of the invention, in particular C1-multimers, can be used for highly efficient removal or enrichment of necrotic and apoptotic cells or debris from or in a cell suspensions or sample (see FIGS. 6A-6D, 10A, 10B, 11A, and 11B). Hence, the present invention also relates to an in vitro method of depleting PS-positive dead or dying cells, cell debris, activated thrombocytes and/or extracellular vesicles from a sample comprising living cells or enriching PS-positive dead or dying cells, cell debris, activated thrombocytes and/or extracellular vesicles in a sample comprising living cells, the method comprising a) contacting said sample with a PS binding agent as defined elsewhere herein, b) contacting said sample with a label capable of binding to the PS binding agent, and c) depleting or enriching PS-positive dead or dying cells, cell debris and/or extracellular vesicles bound to the PS binding agent. According to said method, the PS-positive dead or dying cells, cell debris, activated thrombocytes and/or extracellular vesicles in a sample comprising living cells are firstly bound by the PS binding agent under conditions allowing said binding, and in the next step said bound PS binding agent is additionally contacted and bound by a label. Said label then allows the depletion of said PS-positive dead or dying cells, cell debris and/or extracellular vesicles bound to said PS binding agent from said sample, or the enrichment of said PS-positive dead or dying cells, cell debris and/or extracellular vesicles bound to said PS binding agent in said sample. “Bound” means in this respect that there is an association or linkage between the PS binding agent and said PS-positive dead or dying cells, cell debris, activated thrombocytes and/or extracellular vesicles in said sample. In particular, the PS binding agent of the present invention binds via the comprised one or more (e.g., at least two) C-domains (having phosphatidylserine (PS)-binding activity) to the PS on the surface of said PS-positive dead or dying cells, cell debris, activated thrombocytes and/or extracellular vesicles. The label used in this context is preferably a fluorophore, a solid phase carrier, preferably a bead, or a microbubble. Said label can bind to said PS binding agent, in particular the additional molecule comprised in said PS binding agent capable of linking said C-domains to each other, under any condition allowing said binding. Methods allowing the binding of said labels to said additional molecule, e.g. streptavidin, have been described elsewhere herein. Preferably, in case of a fluorophore or a bead, said labels are coupled/coated to an antibody targeting said additional molecule comprised in said PS binding agent. The term “capable of binding” means that said label specifically interacts with or binds to said PS binding agent bound to PS-positive dead or dying cells, cell debris, activated thrombocytes and/or extracellular vesicles in said sample via direct or indirect linking defined elsewhere herein.

Alternatively provided is an in vitro method of depleting PS-positive dead or dying cells, cell debris, activated thrombocytes and/or extracellular vesicles from a sample comprising living cells or enriching PS-positive dead or dying cells, cell debris, activated thrombocytes and/or extracellular vesicles in a sample comprising living cells, the method comprising a) contacting said sample with a PS binding agent as defined elsewhere comprising a label, and b) depleting or enriching PS-positive dead or dying cells, cell debris and/or extracellular vesicles bound to the PS binding agent. In this respect the PS binding agent used to contact said sample already comprises a label which is preferably bound to the additional molecule comprised in said PS binding agent capable of linking said C-domains to each other defined elsewhere herein, wherein said label is connected to said additional molecule via direct or indirect linking as described elsewhere herein. The label used in this context is preferably a fluorophore, a solid phase carrier, preferably a bead, or a microbubble.

It is further envisaged that the depletion methods described herein comprise an additional staining step with a fluorophore as defined elsewhere herein. In this regard, the PS binding agent may additionally comprise a fluorophore when contacted with the respective sample comprising said PS-positive dead or dying cells, cell debris and/or extracellular vesicles. Alternatively, said sample comprising PS-positive dead or dying cells, cell debris and/or extracellular vesicles is stained with said fluorophore after being contacted with the PS binding agent. The additional fluorophore allows to control efficiency of the depletion of PS-positive dead or dying cells, cell debris and/or extracellular vesicles by the specified depletion methods.

Hence, the present invention also refers to the use of the PS binding agent as defined elsewhere herein, the composition as defined elsewhere herein, or the kit defined elsewhere herein for depleting PS-positive dead or dying cells, cell debris, activated thrombocytes and/or extracellular vesicles.

The term “depleting or depletion” as used in this respect refers to the removal of dead or dying cells, cell debris, activated thrombocytes and/or extracellular vesicles from a sample comprising living cells without significantly reducing the amount of said living cells in said sample. In other words, after contacting said sample with the PS binding agent of the present invention, the sample still contains the same amount of living cells while dead or dying cells, cell debris, activated thrombocytes and/or extracellular vesicles have been significantly reduced. Hence, the PS binding agents of the present invention can be used to purify samples or suspensions comprising living cells, thereby leading to an enrichment of living cells for research and development. Accordingly, the in vitro methods of depleting PS-positive dead or dying cells, cell debris, activated thrombocytes and/or extracellular vesicles from a sample as described herein improves the quality and applicability, if said samples or suspensions comprising living cells for various diagnostic as well as clinical applications, such as adoptive cell therapy. “Significantly reduced” as used in this respect means that at least about 50% of dead or dying cells, cell debris, activated thrombocytes and/or extracellular vesicles comprised in the sample contacted with the PS binding agent of the present invention have been removed by contacting said sample once with said PS binding agent when compared to the untreated sample, i.e. a sample not yet contacted with the PS binding agent of the present invention. More preferred about 60%, even more preferred about 70%, even more preferred about 80%, even more preferred about 85%, even more preferred about 90%, even more preferred about 95%, even more preferred about 96%, even more preferred about 97%, even more preferred about 98%, even more preferred about 99%, most preferred 100% of the dead or dying cells, cell debris, activated thrombocytes and/or extracellular vesicles comprised in the sample contacted with the PS binding agent of the present invention have been removed when compared to the untreated sample.

The term “enriching or enrichment” as used in this regard means that said PS-positive dead or dying cells, cell debris, activated thrombocytes and/or extracellular vesicles are concentrated in a sample comprising living cells, wherein living cells are removed from said sample. In other words, after contacting said sample with the PS binding agent of the present invention, the number of living cells is significantly reduced from said sample while dead or dying cells, cell debris, activated thrombocytes and/or extracellular vesicles remain and are enriched therein, and can subsequently be used for further applications. “Significantly reduced” as used in this respect means that at least about 50% of the living cells comprised in the sample are removed after contacting said sample with the PS binding agent of the present invention when compared to the untreated sample, i.e. a sample not yet contacted with the PS binding agent of the present invention. More preferred about 60%, even more preferred about 70%, even more preferred about 80%, even more preferred about 85%, even more preferred about 90%, even more preferred about 95%, even more preferred about 96%, even more preferred about 97%, even more preferred about 98%, even more preferred about 99%, most preferred 100% of the living cells comprised in the sample are removed after contacting said sample with the PS binding agent of the present invention when compared to the untreated sample. Hence, the PS binding agents of the present invention can also be used to enrich the amount of PS-positive dead or dying cells, cell debris, activated thrombocytes and/or extracellular vesicles in a samples or suspensions comprising living cells for research and development.

It is also envisaged in this respect that the sample to be depleted from PS-positive dead or dying cells, cell debris, activated thrombocytes and/or extracellular vesicles comprising living cells is contacted several times with said PS binding agents described herein until the desired reduction of dead or dying cells, cell debris, activated thrombocytes and/or extracellular vesicles has been reached. Equally, it is envisaged that the sample to be enriched with PS-positive dead or dying cells, cell debris, activated thrombocytes and/or extracellular vesicles comprising living cells is contacted several times with said PS binding agents described herein until the desired enrichment of said dead or dying cells, cell debris, activated thrombocytes and/or extracellular vesicles has been reached. In this context, the compositions or the kits as defined elsewhere herein comprising the PS binding agent of the present invention are applicable for said depletion method.

The term “contacting” as used in this context of the methods described herein means that the sample is brought together with the PS binding agent as defined herein under condition allowing a binding of said PS binding agent to said PS-positive dead or dying cells, cell debris, activated thrombocytes and/or extracellular vesicles. The terms “contacting”, “bringing into contact”, or “bringing together” as used herein, are not particularly limited and include all means of contacting cells/tissues/samples with the PS binding agent of the present invention. For example, for in vitro applications, the PS binding agent of the invention can be added to suspensions or samples in which cultured cells/tissue are kept.

As set forth above, samples or suspensions having been contacted with the PS binding agent of the present invention can be additionally contacted with a label capable of binding to the PS binding agent bound to PS-positive dead or dying cells, cell debris, activated thrombocytes and/or extracellular vesicles, as described elsewhere herein, e.g. beads or fluorophores coupled to an antibody targeting the PS binding agent, thereby allowing the binding of the antibody to the PS binding agent. When a label binds to the PS binding agent according to the present invention, said label is capable of targeting and interacting with said binding agent and thereby marks said PS binding agent, which is bound to PS-positive dead or dying cells, cell debris, activated thrombocytes and/or extracellular vesicles in said sample or suspension. The term “incubate” can be used as a synonym for “contact”. That means that the sample is first contacted with a PS binding agent as defined elsewhere herein for binding of said PS binding agent to said PS-positive dead or dying cells, cell debris, activated thrombocytes and/or extracellular vesicles, and afterwards said marked cells are further contacted with a label capable of binding to the PS binding agent bounds to PS-positive dead or dying cells, cell debris, activated thrombocytes and/or extracellular vesicles, such as beads or fluorophores coupled to an antibody targeting the PS binding agent already bound by said PS-positive dead or dying cells, cell debris, activated thrombocytes and/or extracellular vesicles or microbubbles. Alternatively, said samples or suspensions can be incubated with a PS binding agent already comprising a label bound to said additional molecule comprised in said PS binding agent, such as a bead, a fluorophore or a microbubble as described elsewhere herein. In this case, the PS binding agent has been previously linked or coupled to said label and can directly be used for contacting said PS-positive dead or dying cells, cell debris, activated thrombocytes and/or extracellular vesicles in said sample or suspension. The respective “incubation time” or “contacting time” refers to the time required for the binding of two types of molecules to achieve equilibrium, i.e. for (i) the binding of a PS binding agent as defined elsewhere herein to PS-positive dead or dying cells, cell debris, activated thrombocytes and/or extracellular vesicles, and (ii) for the binding of beads coupled to an antibody to the PS binding agent already bound on PS-positive dead or dying cells, cell debris, activated thrombocytes and/or extracellular vesicles in said sample, wherein said incubation times can be different.

The final removal or enrichment of said dead or dying cells, cell debris, activated thrombocytes and/or extracellular vesicles bound to the PS binding agent of the present invention can be performed using methods known to those skilled in the art, such as magnetic activated cell sorting (MACS), Fluorescent activated cell sorting (FACS), gravity flow or centrifugation.

In accordance with the present invention, the terms “determining”, “measuring”, “evaluating”, “assessing” and “assaying” are used interchangeably and include determining if an element is present or absent. Any suitable form of analysis can be employed in this regard. These terms further include quantitative determinations. Assessing may be relative or absolute. “Determining the presence of” includes determining the amount of something present, as well as determining whether it is present or absent.

As demonstrated in FIGS. 74 and 7B, the PS binding agent of the present invention can also be used to detect cell-associated PS positive vesicles in animals such as mice to whom the PS binding agent has been administered/pre-delivered, wherein the analysis of a sample obtained from said animal is performed in vitro. Hence, in another aspect the present invention also provides for an in vitro method of detecting PS-positive dead or dying cells, cell debris, activated thrombocytes and/or extracellular vesicles, preferably PS-positive cell-associated extracellular vesicles in an animal to whom a PS binding agent as defined elsewhere herein comprising a fluorophore has been pre-delivered, comprising a) analyzing a sample obtained from said animal, b) detecting said pre-delivered PS binding agent using an imaging method, thereby collecting imaging data, c) comparing the imaging data received in step b) to reference imaging data. Said animal is preferably a mammal, such as a non-human primate, a rat, a guinea pig, a rabbit, a cat, a dog, a monkey, a horse or a mouse. Most preferred said animal is a mouse. The administration of the PS binding agents in vivo to animals and the subsequent in vitro analysis of a sample obtained from said animal may be particularly envisaged for the analysis or detection of PS-positive dead or dying cells, cell debris, activated thrombocytes and/or extracellular vesicles in various animal models for research and development purposes. Hence, also provided herein is a method of detecting PS-positive dead or dying cells, cell debris and/or extracellular vesicles, preferably PS-positive cell-associated extracellular vesicles in an animal, preferably a mouse, comprising a) administering a PS binding agent as defined elsewhere herein comprising a fluorophore to said animal, b) obtaining a sample from said animal, c) detecting said pre-delivered PS binding agent in said sample using an imaging method, thereby collecting imaging data, and (d) comparing the imaging data received in step c) to reference imaging data.

In this respect, an increased signal in the imaging data from the animal as compared to a reference imaging data indicates the presence of PS-positive dead or dying cells, cell debris, activated thrombocytes and/or extracellular vesicles, preferably PS-positive cell-associated extracellular vesicles in said animal. Alternatively, no difference in the imaging signal in the imaging data from the animal as compared to a reference imaging data indicates no presence of PS-positive dead or dying cells, cell debris, activated thrombocytes and/or extracellular vesicles, preferably PS-positive cell-associated extracellular vesicles in said animal.

Accordingly, this method of detecting or imaging PS-positive dead or dying cells, cell debris, activated thrombocytes and/or extracellular vesicles, preferably PS-positive cell-associated extracellular vesicles in the body of said animal as defined herein does not comprise an active administration of any of the PS binding agent according to the present invention to said animal, but refers to a situation, where any of the PS binding agents according to the present invention has been pre-delivered to said animal. “Pre-delivered” includes in this regard, that the PS binding agent of the present invention comprising a fluorophore has been delivered to the animal prior to the methods and uses of the present invention (and all associated embodiments), i.e. before the methods of the invention are to be carried out. “Analyzing” means that said sample from said animal is examined for the intended purpose, i.e. the detection of PS-positive dead or dying cells, cell debris, activated thrombocytes and/or extracellular vesicles. Imaging methods applicable in this respect have been described elsewhere herein. Said imaging method is preferably an optical imaging method applicable in this respect as described elsewhere herein for non-invasive imaging methods.

The terms “detecting or detection” when used throughout various aspects of the present invention refers to the visualization and the qualitative analysis of the presence or absence of PS-positive dead or dying cells, cell debris, activated thrombocytes and/or extracellular vesicles, preferably PS-positive cell-associated extracellular vesicles in vitro using the compounds of the present invention and any of the imaging techniques described elsewhere herein. In the context of an in vitro method of detecting PS-positive dead or dying cells, cell debris, activated thrombocytes and/or extracellular vesicles, preferably PS-positive cell-associated extracellular vesicles in a subject to whom a PS binding agent as defined elsewhere herein has been pre-delivered, the sample may be any biological sample taken from said subject and being appropriate to detect PS-positive dead or dying cells, cell debris, activated thrombocytes and/or extracellular vesicles, preferably PS-positive cell-associated extracellular vesicles. Non-limiting examples of useful samples may include blood samples, tissues samples, body fluid samples, skin samples or any other samples known to those skilled in the art for use in in vitro diagnosis.

The present invention also refers to a method of producing the PS binding agent comprising at least three C-domains of MFG-E8 protein as defined elsewhere herein, the method comprising a) contacting at least three C-domains of a MFG-E8 protein with an additional molecule capable of linking said C-domains to each other under conditions allowing the formation of a PS binding agent comprising at least three C-domains of MFG-E8 protein, and optionally b) recovering the produced PS binding agent.

The C-domains of a MFG-E8 protein and the additional molecule capable of linking said C-domains to each other under conditions allowing the formation of a PS binding agent comprising at least three C-domains of MFG-E8 protein which are applicable in said method have been defined elsewhere herein and are applicable for said method. Kits which enable the skilled person to produce yourself the multimeric PS binding agent as defined herein comprising at least three C-domains of a MFG-E8 protein and an additional molecules capable of linking said C-domains to each other under conditions allowing the formation of a PS binding agent have been defined elsewhere herein can equally be applied in said method. In this respect reaction buffers as described elsewhere herein which allow the formation of said PS binding agent comprising at least three C-domains of MFG-E8 protein are applied.

The at least three C-domains of a MFG-E8 protein refer to monomeric C-domains defined elsewhere herein that are linked (either directly or indirectly) to each other when contacted with said additional molecule. In this context, the terms “contacting”, “bringing into contact”, or “bringing together” as used herein, are not particularly limited and include all means of contacting said C-domains as used in said method of producing with said additional molecule of the present invention, preferably in a suitable reaction buffer/solution as it is defined elsewhere herein for said composition/kits. Thus, the end product of the provided production method is the multimeric PS binding agent of the present invention which comprises said at least three C-domains of a MFG-E8 protein. As described elsewhere herein, said additional molecule may not be able to directly link said C-domains to another. When the molecule is capable of linking said C-domains indirectly to each other, said C-domains are multimerized using said additional molecule. Preferably, said additional molecule is streptavidin or avidin as described elsewhere herein.

Said method of producing the PS binding agent comprising at least three C-domains of MFG-E8 protein may further comprise a step of enzymatic modification of said C-domains as described elsewhere herein. Preferably, said method makes use of C-domains comprising a recognition sequence for said enzyme as defined elsewhere herein, so that said enzyme can modify said C-domains before being contacted with said molecule capable of linking said C-domains. The disclosure for said recognition sequences as defined elsewhere herein may be applicable in this regard. Preferably, the method comprises the use of a biotin ligase or a sortase A for said enzymatic modification of said C-domains. Said biotin ligase is preferably BirA. In a preferred embodiment, the method comprises the use of sortase A for said enzymatic modification of said C-domains. Again, the disclosure for said particular enzymes as defined elsewhere herein may be applicable in this respect. Said enzymes catalyzes the biotinylation of said at least three C-domains which are subsequently contacted with said additional molecule capable of linking said C-domains to each other allowing the formation of a PS binding agent comprising at least three C-domains of MFG-E8 protein.

After formation of said PS binding agent comprising at least three C-domains of MFG-E8 protein, the method may further comprise the recovering of the produced PS binding agent. The term “recover” or “recovering” when used in this respect can be understood in that the produced PS binding agent comprising at least three C-domains of MFG-E8 protein is extracted from the used reaction buffer after formation of and potentially purified before said PS binding agent can be used for further modifications, such as linking said PS binding agent to a label or a drug as described elsewhere herein.

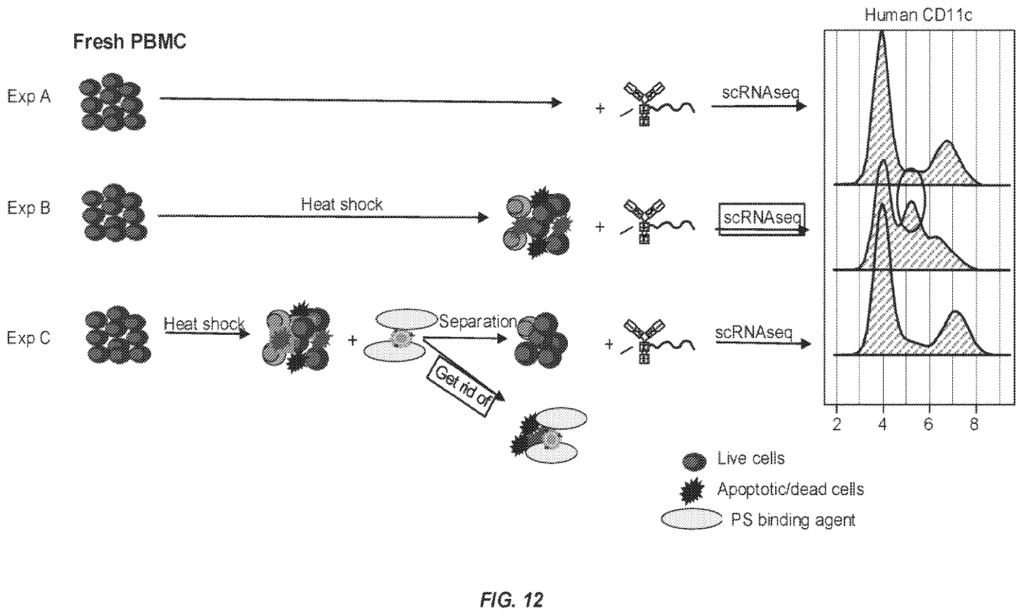

In some of any embodiments, any of the PS binding agents provided herein can be used in the characterization of single cells by measurement of gene-expression levels and cellular proteins. In some embodiments, the PS binding agents described herein can be used to remove dead cells or cell debris prior to the characterization of single cells by measurement of gene-expression levels and cellular proteins, in order to improve data quality (i.e., FIG. 12). Examples of single cell sequencing platforms include CITE-seq and the Drop-seq method, including, but not limited to, microfluidic, plate-based, or microwell, Seq-Well™ method and adaptations of the basic protocol, and InDrop™ method.

In another embodiment, a single cell sequencing platform is 10× genomics single cell 3′ solution or single cell V(D)J solution, either run on Chromium controller, or dedicated Chromium single cell controller. Other suitable sequencing methods include Wafergen iCell8™ method, Microwell-seq method, Fluidigm CI™ method and equivalent single cell products. Still other known sequencing protocols include BD Resolve™ single cell analysis platform and ddSeq (from Illumina® Bio-Rad® SureCell™ WTA 3′ Library Prep Kit for the ddSEQ™ System, 2017, Pub. No. 1070-2016-014-B, Illumina Inc., Bio-Rad Laboratories, Inc.). In still other embodiment, the sequencing platforms include combinatorial indexing based approaches (sci-RNA-seq™ method or SPLiT-seq™ method) and Spatial Transcriptomics, or comparable spatially resolved sequencing approaches. The methods and compositions described herein can also be used as an added layer of information on standard index sorting (FACS) and mRNA-sequencing-based approaches.

### EXAMPLES

The following Examples illustrate the invention, but are not to be construed as limiting the scope of the invention.

### Example 1—Experimental Protocols and Assays

Material and Methods

Mice

C57BL/6 mice were analyzed in sex and age-matched groups of 8-10 weeks of age. The SPF status of the facility was tested according to the Federation for Laboratory Animal Science Associations (FELASA) recommendations. Animal experiment permissions were granted by the animal ethics committee of the Regierung von Oberbayern, Munich, Germany. All mice were bred and maintained at the animal facility of the Institute for Immunology, Ludwig-Maximilians-Universitat Munchen.

Human Studies

All procedures and the selection of volunteers were approved by the Ethics Committee of the Ludwig-Maximillians-Universität München. Informed consent was obtained from all donors. PBMCs were purified from 7 mL of EDTA anti-coagulated blood samples by mixing with an equal amount of PBS, layering onto 14 mL of Pancoll solution (PAN Biotech) and centrifugation. Then PBMCs were collected at the interface, washed and stained with indicated antibodies.

Cloning

C1- and C2-proteins were cloned as GST-SUMO3-fusions to contain at the 3′-end a sequence coding for GSLPETGGSLE (sortase recognition site, bold, SEQ ID NO: 13) (SEQ ID NO: 12) and a His₆ tag. The expression plasmids for GST-SUMO-C1 and -C2 were generated by cutting pGEX-4T-1 with EcoRI and inserting the SUMO3 and C1 sequence (SEQ ID NO.: 1) or C2 sequence (SEQ ID NO.: 2) by Gibson Assembly using overlapping ends which were generated by PCR amplification from the pETM11-SUMO3-GFP plasmid (EMBL) or mMFG-E8 sequence (synthesized by Eurofins). The fragments coding for C1 and C2 were extended at the 3′-end with a sequence coding for GSLPETGGSLE (sortase recognition site, bold, SEQ ID NO: 13) (SEQ ID NO: 12) and His₆ tag.

Expression and Purification of C1- and C2- Domains

A culture of Origami B, transformed with the plasmid coding for GST-SUMO3-(C1/C2)-LPETG (SEQ ID NO: 13)-His₆ was grown in LB medium with the respective antibiotics (100 μg/ml Ampicillin, 50 μg/ml Kanamycin, 10 μg/ml Tetracyclin, (Carl Roth)) overnight shaking at 37° C. The next day, bacteria were pelleted, transferred to 500 ml fresh medium and grown for 8 h. Cells from the culture were transferred to 11 fresh medium, protein expression induced by addition of 1 mM IPTG (Thermo Fisher Scientific) and cultures grown shaking at 25° C. for 15 h. Pellets from expression cultures were resuspended in 10 ml/g wet cell weight lysis buffer (20 mM HEPES, 300 mM NaCl, 20 mM imidazole, pH 7.4, supplemented for cell lysis with 1× protease inhibitor cocktail (Roche complete)) and lysed using a Branson sonicator B15. Lysate was spun 30,000×g, 30 min, 4° C., soluble supernatant added to 4 ml Protino Ni-NTA affinity chromatography beads (Macherey-Nagel) and rotated at 4° C. for at least 1 h. Beads were washed with wash buffer (lysis buffer+0.1% Triton X-100) and lysis buffer w/o protease inhibitors in gravity flow chromatography column. On-bead cleavage of GST-SUMO3 tag with 2×250 μg GST-Senp2 was performed for 1.5 h each rotating at room temperature in lysis buffer. GST-Senp2 was previously produced in BL21 (DE3), purified under standard conditions using Glutathione Sepharose 4B (GE Healthcare) and eluted with 10 mM reduced L-glutathione (Carl Roth). After cleavage with GST-Senp2, beads were washed and protein eluted with in five fractions. Protein-containing fractions were pooled, concentrated and loaded on a HiLoad 26/600 Superdex 200 pg column for size exclusion chromatography.

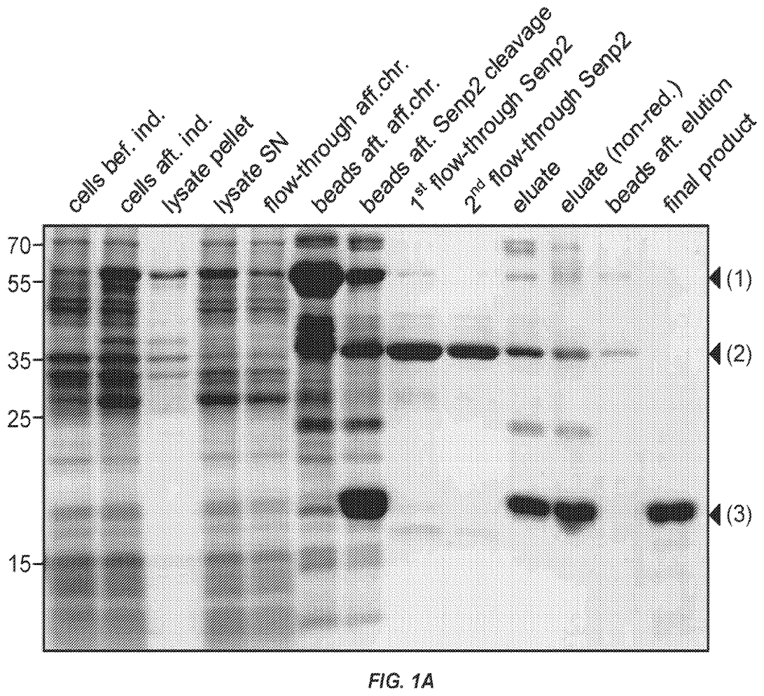

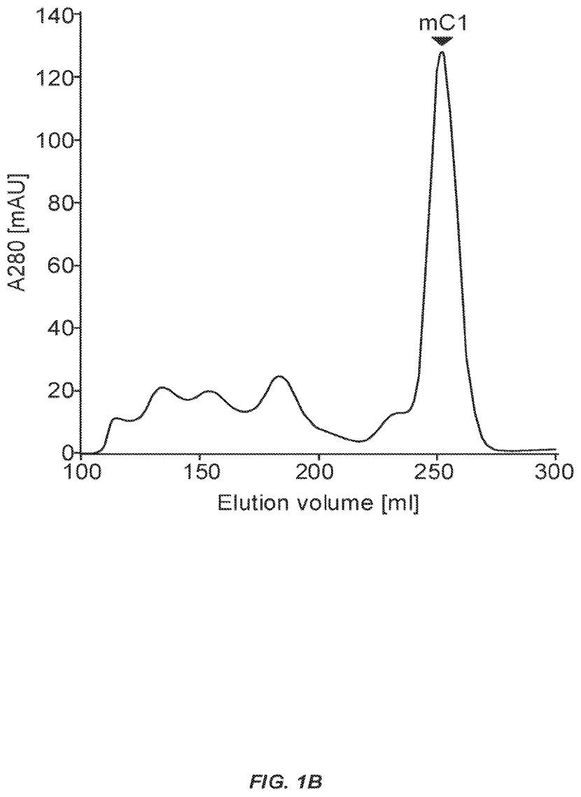

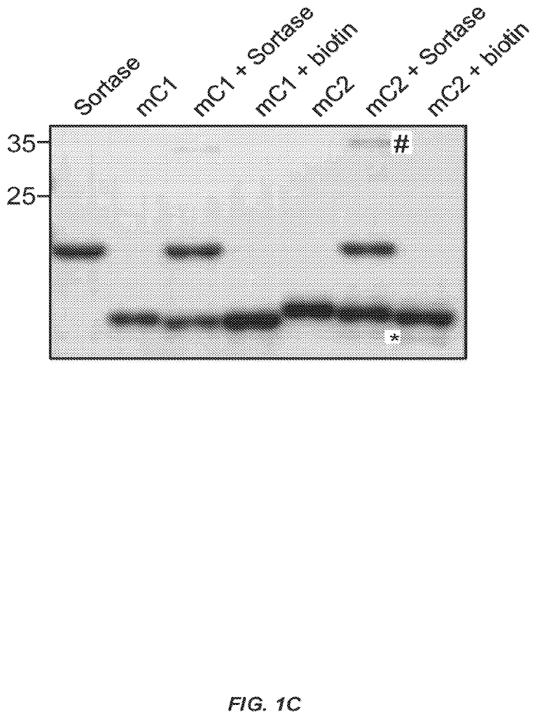

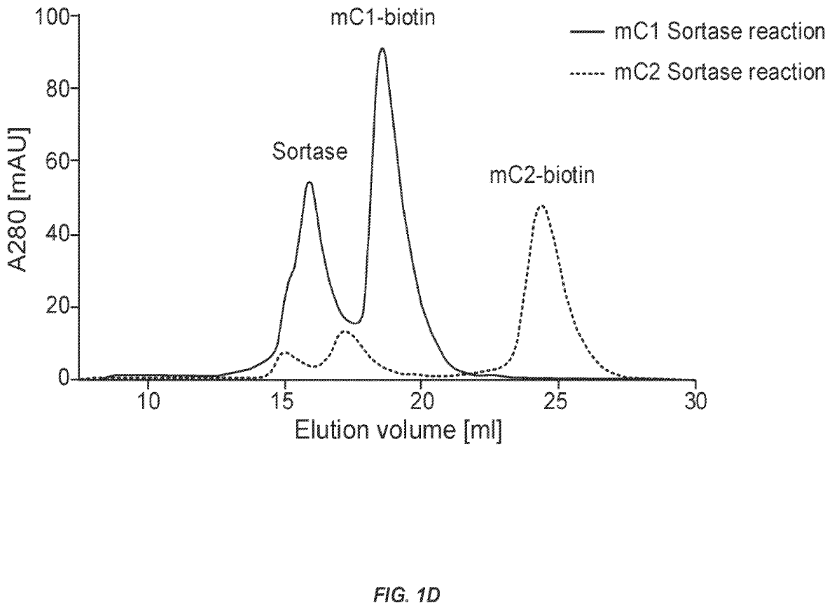

FIGS. 1A-1D show that mMFG-E8 single domains can be expressed in E. coli and biotinylated in a sortase-reaction.

### Lysis of Bacteria

Lysis of Origami B (DE3) cells expressing the mC1-LPETG (SEQ ID NO: 13)- His₆ and mC2-LPETG (SEQ ID NO: 13)-His₆ construct was performed in 50 mM HEPES, 400 mM NaCl, 10% glycerol, pH 7.6. For lysate supernatant after centrifugation, fluorescence was determined with NanoDrop ND- 1000 Spectrophotometer (peqlab; Erlangen) at 488 nm.

Protein Purification

Protein solution was concentrated to 5 ml and loaded on a HiLoad 26/600 Superdex 200 pg column (GE Lifesciences; Chicago, Illinois, USA), preequilibrated with stabilization buffer (25 mM HEPES, 500 mM NaCl, 8% glycerol, 18 mM L-arginine, 3.5 mM L-leucine, 5.7 mM L-glutamic acid, pH 7.4). Protein-containing fractions were pooled and protein concentrated up to 5 mg/ml. Prepared aliquots and stored at −80° C.

Biotinylation

Biotin-containing peptide GGG-K(biotin) was purchased from Genscript Biotech (Piscataway Township, New Jersey, USA). 50 μM target protein was combined with 50 μM sortase A, 1 mM peptide and 1× stabilization buffer. Incubation of reaction for at least 1 h at 25° C. Reaction was stopped by addition of 3 parts stop buffer (stabilization buffer with 7 mM EDTA, 3× complete protease inhibitor (Roche), pH 7.4 at 4° C.) to 1 part sortase reaction, incubation for 20 min on ice. Excess peptide was removed by repeated concentration in VIVASPIN 20, PES membrane, 10 kDa MWCO (Sartorius) and dilution with sortase stop buffer w/o protease inhibitor until the calculated peptide concentration was <0.004 μM. Concentrated sortase reaction mix was loaded on HiLoad 26/600 Superdex 200 pg column for analytical or small-scale size exclusion chromatograpy with Superdex 200 Increase 10/300 GL. Concentration of fractions with target protein was performed using VIVASPIN concentrators, protein concentration was determined with NanoDrop ND-1000 (peqlab) and protein stored at −80° C.

Multimer Assembly

The stoichiometric ratio of C1-biotin/C2-biotin and SA was calculated based on protein concentrations obtained by spectrophotometry (NanoDrop ND-1000; peqlab; Erlangen) using the respective molar extinction coefficient for C1-biotin/C2-biotin or concentrations as provided by the manufacturer for Avidin. Four accessible biotin binding sites per molecule were assumed. For complete assembly, biotinylated monomers were used in at least 4.5× molar excess to SA. The mix was incubated for at least 10 min on ice for assembly. Long-term storage after centrifugation 30000×g, 30 min, 4° C. and snap-freezing in aliquots at −80° C. Avidin variants for multimer assembly were Strepavidin(FITC) and Streptavidin(AlexaFluor647) (both BioLegend; San Diego, California, USA), Streptavidin (NEB; Ipswich, Massachusetts, USA).

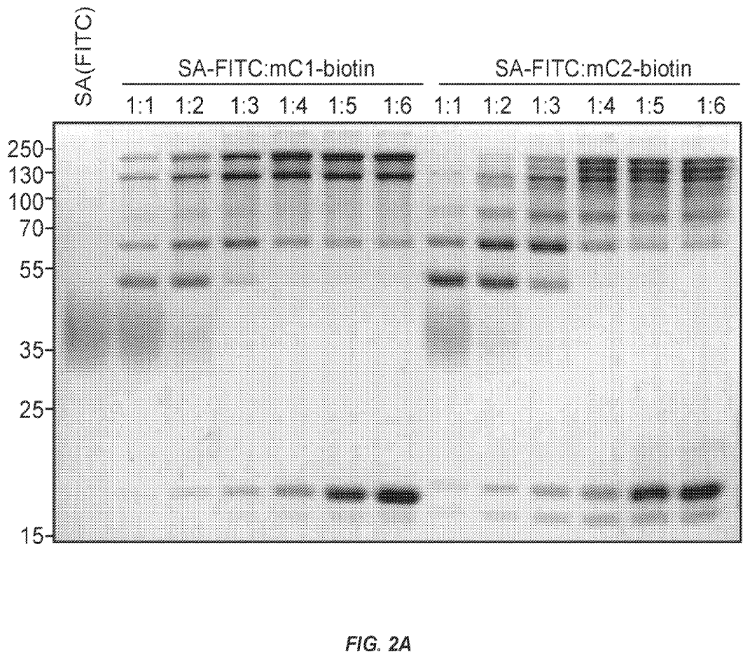

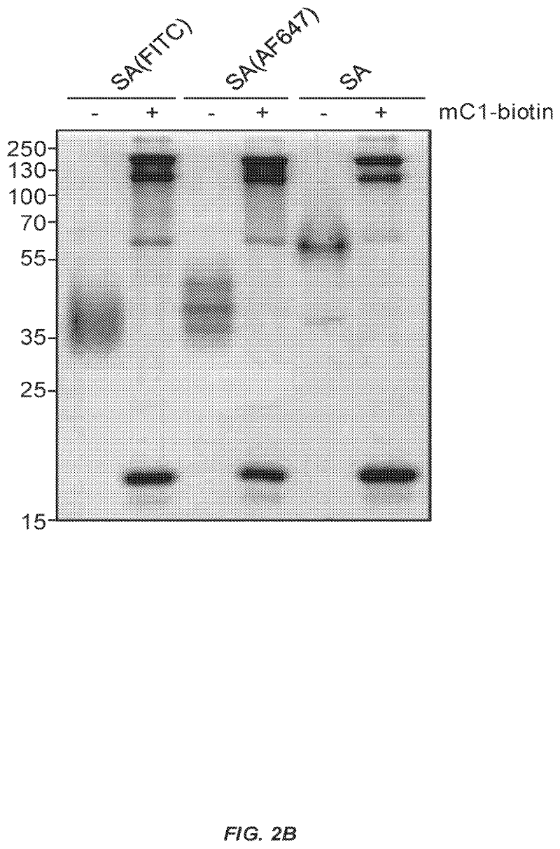

FIGS. 2A and 2B show fully assembled multimers with distinct band patterns independent of the used SA variant.

In Vivo Application of C1-Multimers

Due to the high NaCl₂ concentration in the stabilization buffer, the buffer system had to be changed prior to injection of C1-multimers into mice. For this, C1-multimers were diluted in a large volume of injection buffer (25 mM HEPES, 2% Glycerol, 200 mM L-Arginine, 200 mM L-Glutamic Acid, 150 mM NaCl) and reconcentrated to the initial volume using VIVASPIN 20, PES membrane, 10 kDa MWCO (Sartorius) spin columns.

Induction of Cell Death in Thymocytes

Thymocytes were prepared from fresh thymi and erythrocytes lysed for 5 min at room temperature using ACK lysis buffer. After washing with FACS buffer (PBS+2% FCS), cells were resuspended in DMEM GlutaMAX™ medium (Thermo Fisher Scientific) -FCS+1% Penicillin/Streptomycin (Life Technologies) and either left untreated on ice or 30×10 6 cells incubated for 2 h with 0.2 μg/ml staurosporine (Sigma) in a petri dish with 10 ml DMEM -FCS+1% Penicillin/Streptomycin in an atmosphere with 37° C. and 5% CO2. Subsequently, cells were washed twice with FACS buffer and stored overnight on ice at 4° C.

Dead Cell Depletion Assay

Anti-SA antibody (clone 3A20.2, #410501, BioLegend) was coupled to tosylactivated Dynabeads M-450 (#14013, Thermo Fisher Scientific) according to manufacturers' instructions. Prepared mouse splenocytes, removed erythrocytes via ACK lysis (5 min on room temperature with 1 ml of 150 mM NH₄Cl, 10 mM KHCO₃, 0.1 mM Na₂EDTA) and stained 5×10⁵ cells each with fixable viability dye eFluor780 (#65-0865-14, Invitrogen), followed by 10 min incubation in 20 μl Fc block 1:400 (TruStain fcX anti-mouse CD16/32, BioLegend). After staining with 100 μl of 100 ng/ml C1 multimer (FITC) or 100 ng/ml purified mMFG-E8-EGFP and washing with 2×100 μl FACS buffer, cells were added to 200 μl beads in 600 μl total volume and rotated slowly at 4° C. for 40 min. Cells were carefully spun (300×g, 4° C., 5 min) and beads separated from supernatant using a magnetic rack. Beads were washed with 2×200 μl and collected supernatant spun again, resuspended in a smaller volume and analyzed by flow cytometry.

FIGS. 6A-6D show that C1-multimers can be utilized to remove dead cells from cell suspensions.

Monomer/Multimer Comparison for C1-Biotin and C2-Biotin

Biotinylated C1 and C2 in five-fold molar excess were mixed with SA(FITC) (BioLegend) or the same amount left as monomer. Incubated for 1 h at room temperature for multimer assembly, took samples and mixed with 2× SDS sample buffer (reducing) for SDS gel analysis. Diluted the rest with FACS buffer, snap-froze and stored at −80° C. For FACS analysis, same amounts were serially diluted in 96 well plate and 1×10⁶ thymocytes incubated with monomer and multimer solution. After washing twice, 100 μl of 2.5 μg/ml SA(FITC) was added to monomer-associated cells and incubated again for 30 min on ice.

FIGS. 3A-3C show that C1- and C2-multimers are capable of staining dead cells.

Flow Cytometry

Cells were plated in wells of round-bottom 96 well plate, washed once with PBS and stained for 10 min on ice with 100 μl of a 1:2000 dilution fixable viability dye eFluor780 (#65-0865-14, Invitrogen) in PBS. After addition of 100 μl FACS buffer and removal of supernatant, cells were stained with the respective reagent in 100 μl volume for 30 min on ice. 100 μl FACS buffer was added and cells washed twice before they were finally resuspended for flow cytometry analysis with CANTO II (BD Biosciences).

LCMV Infections

LCMV Armstrong was propagated on L929 cells. Stocks were frozen at −80° C. For quantitation of virus titres focus-forming assays using Vero cells were per-formed as described previously (Pellegrini, Calzascia et al. 2011, Cell 144(4): 601-613). For injections, viral stocks were diluted in sterile PBS. 2×10⁵ p.f u. were injected intraperitonially per mouse.

Preparation of Single Cells Suspensions

Single cell suspensions of spleen and thymocytes were prepared by meshing organs through a 100 μm nylon mesh. Erythrocytes were removed by centrifugation through a Pancoll cushion (Pancoll, PAN Biotech). Number of live cells was determined using a CASY cell counter (OMNI Life Science).

Imaging Flow Cytometry and Data Analysis

5×10⁶ cells were stained with appropriate antibodies for 20minoniceinPBS+2% FCSandanalysedonan ImageStreamX MKII imaging flow cytometer (Merck). C1-multimer+ cells were gated using the IDEAS soft-ware. Then TIF-images of C1-multimer+ cells from each sample were exported (16-bit, raw) and analyzed by the CAE algorithm. The results were stored in two separated *.pop files containing the object numbers of apoptotic and EV⁺ cells. These object numbers were re-imported into IDEAS and two separate files containing only apoptotic or EV⁺ cells were generated. Next, from each sample, three files (containing either all cells, only apoptotic or only EV⁺ cells) were exported as fcs-files which were then further analysed using FlowJo. Deep learning analysis was performed as described previously (Kranich, Chlis et al. 2020, J Extracell Vesicles 9(1): 1792683).

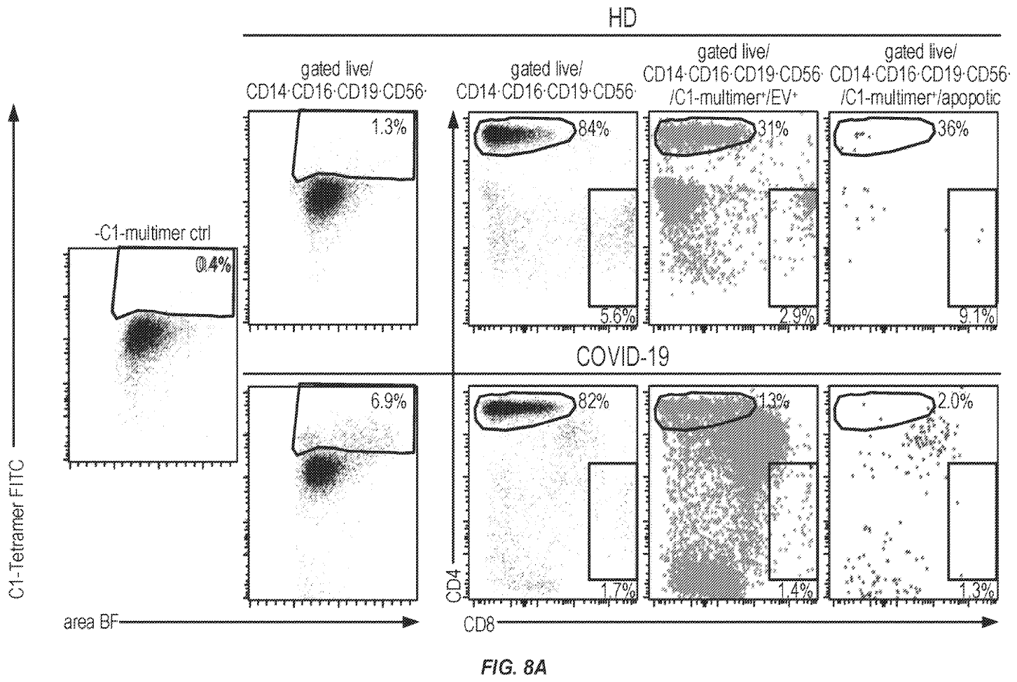

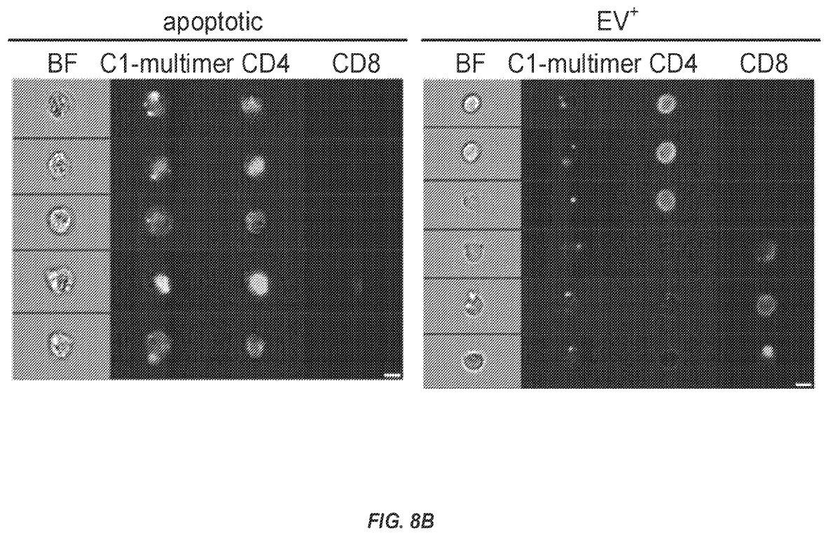

FIGS. 4A and 4B show that mC1 multimers displayed enhanced binding properties over mC2 multimers. FIGS. 5A and 5B show that C1-multimers, Annexin V and mMFG-E8 are capable of staining apoptotic cells. Further, FIGS. 7A and 7B show the identification of dying cells and EV⁺ cells with C1-multimers in vivo and FIGS. 8A and 8B show the identification of dying cells and EV⁺ cells in human blood samples

Generation of AviTagged Mouse MFG-E8 C1 Domain

Design of AviTagged Mouse MFG-E8 C1 Domain

The sequence of the C1 domain (Cys₁₄₈-Cys₃₀₃) of mouse MFG-E8 (UniProtKB-P21956 (MFGM_MOUSE)) was expressed in E. coli. For higher expression and purification purposes, a 6x-His tag and human SUMO (Ala₂-Gly₉₃) (UniProtKB-P61956 (SUMO2_HUMAN)) was fused at the N-terminus of the MFG-E8 C1 domain with additional serine residues at the C-terminus of SUMO, followed by a twelve amino acid linker (GSAGSAAGSGEF (SEQ ID NO: 6)) before the AviTag™.

Cloning

The constructed amino acid sequence was codon optimized and seamless priming sites were added with the following sequences, TGTTTAACTTTAAGAAGGAGATATACAT (SEQ ID NO: 10) at the 5′ end and TAACTCGAGCACCACCACCACCACCACTGAG (SEQ ID NO: 11) at 3′ end. The resulting sequence was synthesized as a gene fragment (Integrated DNA Technologies, Inc.). The gene fragment was cloned into an E. coli expression vector (BioLegend), called pBL-2, by means of seamless cloning using Gene Art and Assembly Enzyme Mix from (Invitrogen, Cat. #A14606). The resulting expression plasmid was designated as pBL-729.

Expression

For protein expression, chemically competent BL-21 cells (MCLAB, Cat. #BL-21-100) were transformed with pBL-729 by heat shock methods. The transformants were grown on agar supplemented with ampicillin. Ampicillin-resistant colonies were directly inoculated into Studier auto-induction media (F. William Studier, Protein Expression and Purification 41 (2005) 207-234) and grown for 24 hours at 37° C. after inoculation. For optimal growth, one agar plate was used to inoculate two liters of media. After 24 hours, cells were harvested by centrifugation for 30 minutes at 4500 rpm. Cells collected from two liters of media were resuspended in 100 mL of breakage buffer (pH 8.0) containing 50 mM Tris, 50 mM NaCl, and 5% glycerol, and stored at 20° C. freezer overnight before lysis by sonication.

Purification

Cells were thawed in a 37° C. water bath, and treated with 200 μg/mL lysozyme (Millipore Sigma, Cat. #L6876), 0.5 mM AEBSF (AGScientific, Cat. #A1018) and 0.2% Igepal AC-630 (Millipore Sigma, Cat. #I3021). Cells were sonicated for 30 minutes to 1 hour to harvest inclusion bodies. The inclusion bodies were washed with 20 mM Tris buffer at pH 8.0 containing 1% Igepal CA-630 solution followed by pH 8.0 Tris buffer containing 50 mM Tris and 100 mM NaCl, and collected by centrifugation after each washing step. The collected inclusion bodies were solubilized in 10-20 mL of solubilization buffer (pH 8.0) containing 50 mM Tris, 6.0 M Guanidine hydrochloride, 1 mM EDTA, and 20 mM DTT.

Proteins contained in the solubilized inclusion bodies were refolded by dropwise dilution at 4° C. in refolding buffer (pH 8.0) containing 50 mM Tris, 0.1 M NaCl, 0.5 M Arginine, 2 mM reduced Glutathione, and 0.2 mM oxidized Glutathione.

Briefly, the solution of inclusion bodes was diluted with solubilization buffer to 4 to 5 mg/mL, added to the refolding buffer dropwise by syringe pump in a 1:10 final dilution ratio, and incubated for at least 16 hours at 4° C. The solution was dialyzed overnight with at least 20-fold excess volume of Ni-A buffer (pH 7.5) containing 20 mM Tris and 300 mM NaCl. The dialyzed solution was then used for Ni-NTA purification.

Ni-NTA resin (MCLAB, Cat. #NINTA-100) packed with 20 to 40 mL of resin was used to purify the AviTagged-MFG-E8. Proteins were eluted with Ni-A buffer containing 200 mM imidazole buffer. Elution fractions containing AviTagged-MFG-E8, as confirmed by SDS-PAGE analysis, were pooled and the protein concentration measured by Bradford assay. SENP2 (BioLegend) was added into pooled proteins to digest the N-terminal His-Sumo tag at a w/w ratio of SENP2 to AviTagged-MFG-E8 of 1:400. The reaction mixture was dialyzed twice for overnight in 40-fold excess volume of Tris buffer (pH 7.5) containing 20 mM Tris, 150 mM NaCl, and 10% glycerol, and purified once more with the Ni-NTA column. The majority of AviTagged-MFG-E8 without the His-Sumo tag was eluted using 20 mM Imidazole in Ni-A buffer. The proteins were pooled, analyzed using SDS-PAGE, and dialyzed once with a 40-fold excess volume of Tris buffer (pH 7.5) containing 20 mM Tris, 150 mM NaCl, and 10% glycerol.

Prior to biotinylation, the concentration of AviTagged-MFG-E8 was measured at OD₂₈₀. Biotinylation was performed according to published procedures (Michael Fairhead and Mark Howarth, Methods Mol Biol. 2015; 1266: 171-184). Briefly, a mixture containing 10 μL, 1 M magnesium chloride, 11 mg of ATP, 40 μL of 1.0 M Tris buffer (pH 9.5), 4.0 μL 50 μM BirA (BioLegend), 40 μL 5 mM D-Biotin (Millipore Sigma, Cat. #B4501), 200 μL of 200 mM Tris buffer (pH 7.5), and 706 μL of 3X protease inhibitor cocktail in water was added to 1 mL of 80-100 μM AviTagged-MFG-E8 for 24 hours at room temperature. The reaction was quenched by addition of EDTA to 20 mM final concentration. The biotinylated proteins were centrifuged at 4000 rpm for 10 minutes to remove precipitates. The supernatant was filtered using a 0.22 μm filter unit. The biotinylated protein solution was further purified using Hi load 16/600 Superdex 75 pg (Cytiva) on AKTA pure instrument. Efficiency of the biotinylation reaction was assessed on a gel shifting assay with binding on streptavidin. Purity of the biotinylated-MFG-E8 C1 were analyzed by SDS-PAGE.

Dead Cell Depletion Assay

Streptavidin Nanobeads-Based Method

Biotinylated-MFG-E8 C1 was incubated with MojoSort™ Streptavidin Nanobeads (BioLegend, Cat. #480016) and MojoSort™ buffer (BioLegend, Cat. #480017) up to 50 μL in 5 mL polystyrene round bottom FACS tubes (Corning, Cat. #352052) at room temperature for 10 minutes without washing. The MFG-E8 C1-streptavidin particles were suspended with 100 μL of mouse splenocytes (1×10⁸ cells/mL) solution and incubated on ice for 15 minutes. The cell-particle mixtures were diluted with MojoSort™ buffer up to 4 mL, and centrifuged at 300 g for 5 minutes. The pellet was resuspended in 2.5 mL of MojoSort™ buffer and placed into a MojoSort™ magnet (BioLegend, Cat. #480019) for 5 minutes. With the tube still in the magnet, the supernatant was poured into a collection tube. The tube was removed from the magnet and labeled cells were resuspended with 2.5 mL of MojoSort™ buffer and placed back into the magnet for an additional 5 minutes. The supernatant was poured into the same collecting tube. Both supernatant and resuspended labeled cells were centrifuged and the resulting pellets were resuspended with 100 μL of Cell Staining Buffer (BioLegend, Cat. #420201). The cells were stained with cell viability dye Helix NP™ Blue (BioLegend, Cat. #425305) for 5 minutes without washing before collecting the data by FACSCanto II Cell Analyzer (BD Biosciences).

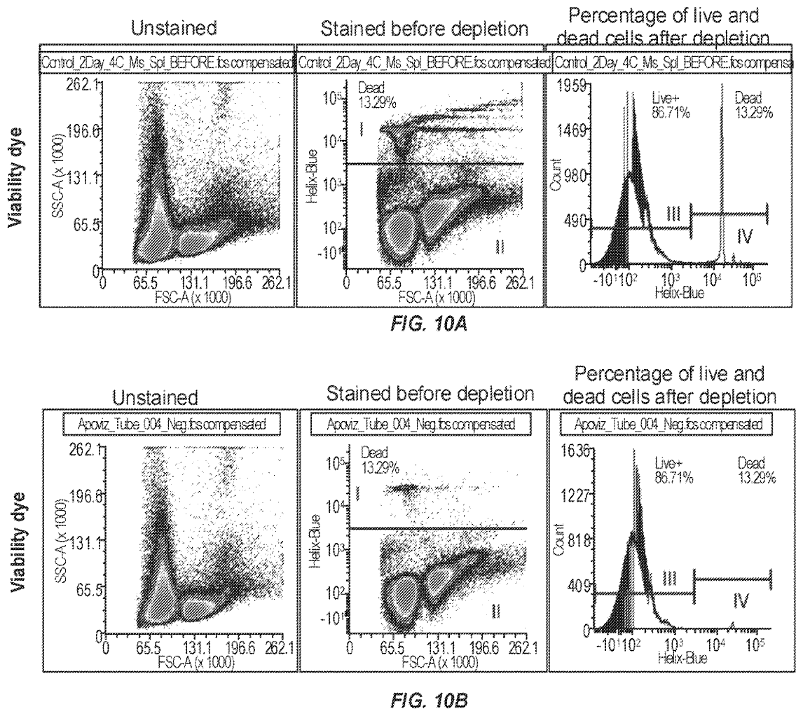

As shown in FIGS. 10A and 10B, magnetic particle—biotin MFG-E8 C1 multimer are capable of being utilized to remove dead cells from cell suspensions.

Streptavidin Microbubbles-Based Method

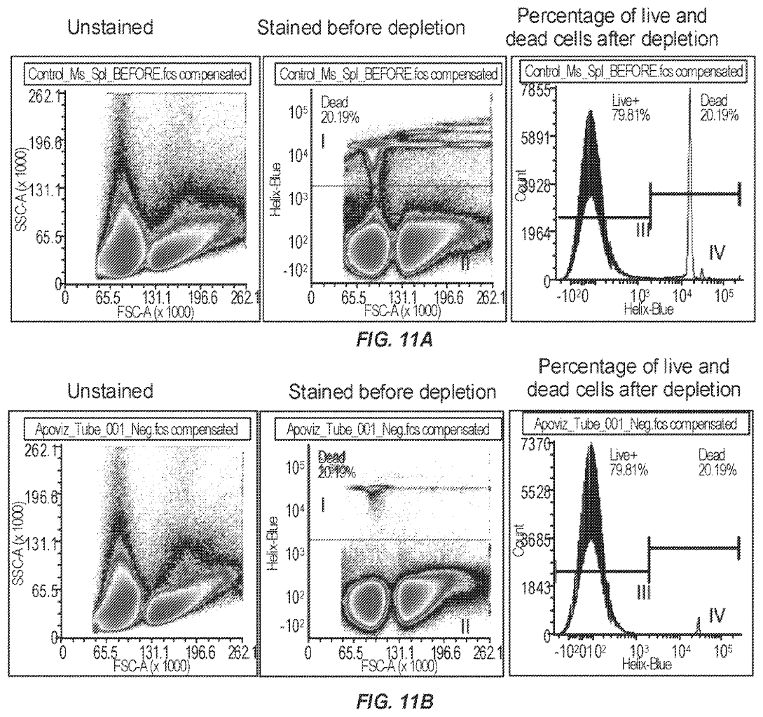

Biotinylated-MFG-E8 C1 was mixed with Streptavidin Microbubbles (BioLegend) in 5 mL Eppendorf tube for 10 minutes at room temperature, gently vortexing intermittently during incubation. The MFG-E8 C1-streptavidin microbubbles mixture was then resuspended with 100 μl of mouse splenocytes (1×10⁸ cells/mL) and pipetted 30 times. The cells-microbubbles were suspended with 3 mL of MojoSort™ buffer (BioLegend, Cat. #480017) and centrifuged at 300 g for 5 minutes at room temperature. The supernatant was removed by aspiration and the resulting pellet was transferred to a 5 mL polystyrene round bottom FACS tubes (Corning, Cat. #352052), diluted with up to 4 mL of MojoSort™ buffer, and centrifuged for 5 minutes. The resulting pellet was resuspended in 100 μL of Cell Staining Buffer (BioLegend, Cat. #420201). The cells were stained with cell viability dye Helix NP™ Blue (BioLegend, Cat. #425305) for 5 minutes before collecting the data by FACSCanto II Cell Analyzer (BD Biosciences). FIGS. 11A and 11B show that microbubble—biotin prepared MFGE-8 C1 multimers can be utilized to remove dead cells from cell suspensions. FIG. 12 further depicts the ability of exemplary prepared MFGE-8 C1 multimers to remove dead cells prior to single cell RNA sequencing. Fresh isolated PBMCs containing 98% live cells (sample A), or samples containing PBMCs spiked with 40% apoptosis induced heat shock treated human PBMCs (Sample B), were stained with TotalSeq™ TBNK lineage antibodies. One sample set containing PBMCs spiked with heat shock treated PBMCs was incubated with exemplary multimers (biotin-MFG-E8 C1 magnetic particle) for 15 minutes, followed by one wash, and separated using a magnetic force (Sample C). All samples were collected and pooled for scRNAseq sequencing. Sample B ridge plots show a middle peak depicting spiked in heat shocked/apoptotic cells, while Sample C does not depict a middle peak, similar to the fresh isolated PBMCs containing 98% live cells in Sample A. This assay suggests the ability of exemplary prepared MFGE-8 C1 multimers to effectively improve data quality in single cell sequencing.

### Example 2—C1-Tetramers Stained CD41⁺ Thrombocytes

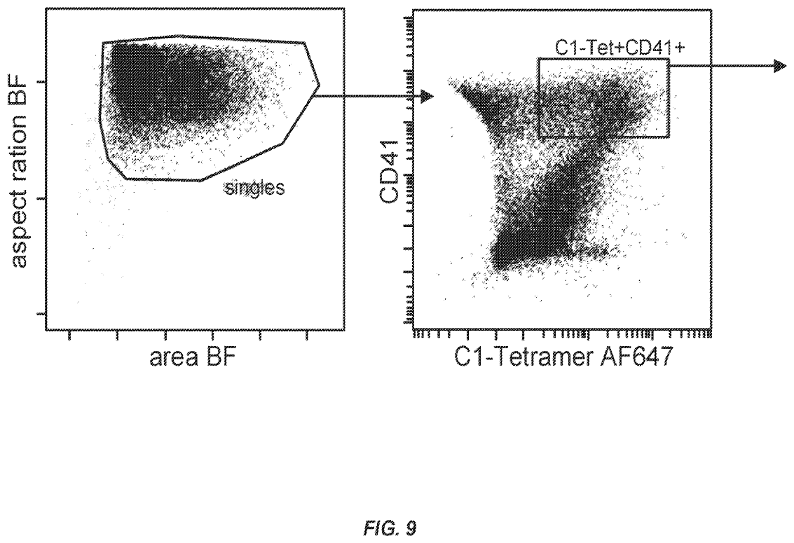

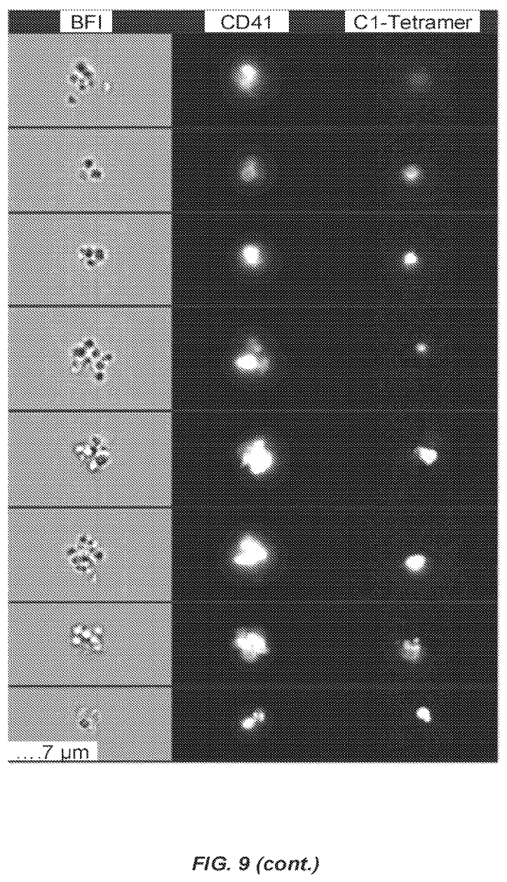

Whole EDTA-blood sample from a lymphoma patient was stained with C1-multimer (SA-AF647) and CD41, a marker for thrombocytes. In FIG. 9, left dot plots show gating of single cells based on the area and aspect ratio of the bright-field signal; right dot plot was gated on single cells and shows expression of CD41 and surface exposure of PS (by staining with C1-tetramer AF647). Images in FIG. 9 show CD41⁺ C1-Tetramer⁺ aggregated platelets.

SEQUENCE TABLE

| SEQ | | |
|---|---|---|
| ID | | |
| NO | Sequence | Notes |
| 1 | CSTQLGMEGGAIADSQISAS | murine C1- |
| | SVYMGFMGLQRWGPELARLY | domain |
| | RTGIVNAWTASNYDSKPWIQ | |
| | VNLLRKMRVSGVMTQGASRA | |
| | GRAEYLKTFKVAYSLDGRKF | |
| | EFIQDESGGDKEFLGNLDNN | |
| | SLKVNMFNPTLEAQYIKLYP | |
| | VSCHRGCTLRFELLGC | |
| 2 | CSEPLGLKNNTIPDSQMSAS | murine C2- |
| | SSYKTWNLRAFGWYPHLGRL | domain |
| | DNQGKINAWTAQSNSAKEWL | |
| | QVDLGTQRQVTGIITQGARD | |
| | FGHIQYVASYKVAHSDDGVQ | |
| | WTVYEEQGSSKVFQGNLDNN | |
| | SHKKNIFEKPFMARYVRVLP | |
| | VSWHNRITLRLELLGC | |
| 3 | MPRPRLLAALCGALLCAPSL | full-length human |
| | LVALDICSKNPCHNGGLCEE | MFG-E8 protein |
| | ISQEVRGDVFPSYTCTCLKG | (Uniprot |
| | YAGNHCETKCVEPLGLENGN | accession number |
| | IANSQIAASSVRVTFLGLQH | Q08431) |
| | WVPELARLNRAGMVNAWTPS | |
| | SNDDNPWIQVNLLRRMWVTG | |
| | VVTQGASRLASHEYLKAFKV | |
| | AYSLNGHEFDFIHDVNKKHK | |
| | EFVGNWNKNAVHVNLFETPV | |
| | EAQYVRLYPTSCHTACTLRF | |
| | ELLGCELNGCANPLGLKNNS | |
| | IPDKQITASSSYKTWGLHLF | |
| | SWNPSYARLDKQGNFNAWVA | |
| | GSYGNDQWLQVDLGSSKEVT | |
| | GIITQGARNFGSVQFVASYK | |
| | VAYSNDSANWTEYQDPRTGS | |
| | SKIFPGNWDNHSHKKNLFET | |
| | PILARYVRILPVAWHNRIAL | |
| | RLELLGC | |
| 4 | MQVSRVLAALCGMLLCASGL | full length mouse |
| | FAASGDFCDSSLCLNGGTCL | MFG-E8 protein |
| | TGQDNDIYCLCPEGFTGLVC | (Uniprot |
| | NETERGPCSPNPCYNDAKCL | accession number |
| | VTLDTQRGDIFTEYICQCPV | P21956) |
| | GYSGIHCETETNYYNLDGEY | |
| | MFTTAVPNTAVPTPAPTPDL | |
| | SNNLASRCSTQLGMEGGAIA | |
| | DSQISASSVYMGFMGLQRWG | |
| | PELARLYRTGIVNAWTASNY | |
| | DSKPWIQVNLLRKMRVSGVM | |
| | TQGASRAGRAEYLKTFKVAY | |
| | SLDGRKFEFIQDESGGDKEF | |
| | LGNLDNNSLKVNMFNPTLEA | |
| | QYIKLYPVSCHRGCTLRFEL | |
| | LGCELHGCSEPLGLKNNTIP | |
| | DSQMSASSSYKTWNLRAFGW | |
| | YPHLGRLDNQGKINAWTAQS | |
| | NSAKEWLQVDLGTQRQVTGI | |
| | ITQGARDFGHIQYVASYKVA | |
| | HSDDGVQWTVYEEQGSSKVF | |
| | QGNLDNNSHKKNIFEKPFMA | |
| | RYVRVLPVSWHNRITLRLEL | |
| | LGC | |
| 5 | CSTQLGMEGGAIADSQISAS | mouse PS-binding |
| | SVYMGFMGLQRWGPELARLY | domain (C1 + C2 |
| | RTGIVNAWTASNYDSKPWIQ | domain) |
| | VNLLRKMRVSGVMTQGASRA | |
| | GRAEYLKTFKVAYSLDGRKF | |
| | EFIQDESGGDKEFLGNLDNN | |
| | SLKVNMFNPTLEAQYIKLYP | |
| | VSCHRGCTLRFELLGCELHG | |
| | CSEPLGLKNNTIPDSQMSAS | |
| | SSYKTWNLRAFGWYPHLGRL | |
| | DNQGKINAWTAQSNSAKEWL | |
| | QVDLGTQRQVTGIITQGARD | |
| | FGHIQYVASYKVAHSDDGVQ | |
| | WTVYEEQGSSKVFQGNLDNN | |
| | SHKKNIFEKPFMARYVRVLP | |
| | VSWHNRITLRLELLGCGSAG | |
| 6 | SAAGSGEF | linker |
| 7 | LPXTG | Sortase A |
| | | recognition |
| | | sequence |
| 8 | LPXTA | Sortase A |
| | | recognition |
| | | sequence |
| 9 | DXPNTGDE | Sortase B |
| | | recognition |
| | | sequence |
| 10 | TGTTTAACTTTAAGAAGGAG | Priming sequence |
| | ATATACAT | at 5′ end |
| 11 | TAACTCGAGCACCACCACCA | Priming sequence |
| | CCACCACTGAG | at 3′ end |
| 12 | GSLPETGGSLE | Sequence at 3′- |
| | | end of C1 and C2 |
| | | fragments in |
| | | cloning |
| 13 | LPETG | Sortase |
| | | recognition |
| | | sequence |

### EXEMPLARY EMBODIMENTS

Exemplary embodiments provided in accordance with the presently disclosed subject matter include, but are not limited to, the claims and the following embodiments:

1. A phosphatidylserine (PS) binding agent comprising one or more isolated C-domains of a milk fat globule-EGF factor 8 (MFG-E8) protein.

2. The PS binding agent of embodiment 1, wherein the one or more isolated C-domains comprises two, three or four or more C-domains of a milk fat globule-EGF factor 8 (MFG-E8) protein.

3. The phosphatidylserine (PS) binding agent of embodiment 1 or embodiment 2, comprising at least two isolated C-domains of a milk fat globule-EGF factor 8 (MFG-E8) protein.

4. The PS binding agent of any one of embodiments 1-3, wherein the one or more isolated C-domains comprise a C1-domain of MFG-E8 protein or a functional fragment thereof, and a C2-domain of MFG-E8 protein or a functional fragment thereof.

5. The PS binding agent of any one of embodiments 1-4, wherein the one or more isolated C-domains comprise a C1-domain of MFG-E8 protein or a functional fragment thereof, or a C2-domain of MFG-E8 protein or a functional fragment thereof.

6. The PS binding agent of embodiment 4 or 5, wherein said C1-domain comprises an amino acid sequence having at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with an amino acid sequence set forth in SEQ ID NO: 1.

7. The PS binding agent of any one of embodiments 4-6, wherein said C1-domain comprises the amino acid sequence set forth in SEQ ID NO: 1.

8. The PS binding agent of any one of embodiments of 4-7, wherein said C2-domain comprises an amino acid sequence having at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with an amino acid sequence set forth in SEQ ID NO: 2.

9. The PS binding agent of any one of embodiments 4-8, wherein said C2-domain comprises the amino acid sequence set forth in SEQ ID NO: 2.

10. The PS binding agent of any one of embodiments 1-9, wherein the one or more C-domains are covalently linked.

11. The PS binding agent of any one of embodiments 1-10, further comprising a recognition sequence for enzymatic modification.

12. The PS binding agent of embodiment 11, wherein the recognition sequence is for a biotin ligase or for a sortase A.

13. The PS binding agent of embodiment 12, wherein the biotin ligase comprises BirA.

14. The PS binding agent of embodiment 12 or embodiment 13, wherein the recognition sequence for sortase A comprises the amino acid sequence set forth in SEQ ID NO: 7 or SEQ ID NO: 8.

15. The PS binding agent of any one of embodiments 1-14, wherein the one or more isolated C-domains are covalently attached to biotin.

16. The PS binding agent of any one of embodiments 1-15, wherein the one or more isolated C-domains are covalently attached to biotin by a sortase A or biotin ligase.

17. The PS binding agent of embodiment 16, wherein the biotin ligase comprises BirA.

18. The PS binding agent of any one of embodiments 1-14, wherein the one or more isolated C-domains are biotinylated.

19. The PS binding agent of embodiment 18, wherein the one or more isolated C-domains are biotinylated with sortase A or a biotin ligase.

20. The PS binding agent of embodiment 19, wherein the biotin ligase comprises BirA.

21. The PS binding agent of any one of embodiments 1-20, wherein the one or more isolated C-domains are linked directly.

22. The PS binding agent of any one of embodiments 1-20, further comprising an additional molecule capable of linking the one or more isolated C-domains.

23. The PS binding agent of embodiment 22, wherein the one or more isolated C-domains are linked indirectly by the additional molecule.

24. The PS binding agent of embodiment 22 or embodiment 23, wherein the additional molecule is selected from streptavidin, avidin or neutravidin.

25. The PS binding agent of any one of embodiments 22-24, wherein the additional molecule comprises streptavidin.

26. The PS binding agent of any one of embodiments 1-25, further comprising a label.

27. The PS binding agent of embodiment 26, wherein the label is selected from the group consisting of a tag, a fluorophore, a solid phase carrier, a microbubble, a linker, and an active site probe.

28. The PS binding agent of embodiment 27, wherein said solid phase carrier comprises a bead.

29. The PS binding agent of any one of embodiments 1-28, wherein the PS binding agent is capable of binding to PS on dead cells, dying cells, cell debris, activated thrombocytes and/or extracellular vesicles.

30. A PS binding agent comprising a backbone and one or more isolated C domains of a MFG-E8 protein.

31. The PS binding agent of embodiment 30, wherein the one or more isolated C-domains comprise a C1-domain of MFG-E8 protein or a functional fragment thereof, and a C2-domain of MFG-E8 protein or a functional fragment thereof.

32. The PS binding agent of embodiment 30 or embodiment 31, wherein the one or more isolated C-domains comprise a C1-domain of MFG-E8 protein or a functional fragment thereof, or a C2-domain of MFG-E8 protein or a functional fragment thereof.

33. The PS binding agent of embodiment 31 or 32, wherein the C1-domain comprises an amino acid sequence having at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with an amino acid sequence set forth in SEQ ID NO: 1.

34. The PS binding agent of any one of embodiments 31 to 33, wherein the C1-domain comprises the amino acid sequence set forth in SEQ ID NO: 1.

35. The PS binding agent of any one of embodiments of 31 to 34, wherein the C2-domain comprises an amino acid sequence having at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with an amino acid sequence set forth in SEQ ID NO: 2.

36. The PS binding agent of any one of embodiments 31 to 35, wherein the C2-domain comprises the amino acid sequence set forth in SEQ ID NO: 2.

37. The PS binding agent of any one of embodiments 31 to 36, wherein the one or more C-domains are covalently linked to the backbone.

38. The PS binding agent of any one of embodiments 30 to 37, wherein the one of more C-domains further comprise a recognition sequence for enzymatic modification.

39. The PS binding agent of embodiment 38, wherein the recognition sequence is for a biotin ligase or for a sortase A.

40. The PS binding agent of embodiment 39, wherein the biotin ligase comprises BirA.

41. The PS binding agent of embodiment 38 or embodiment 39, wherein the recognition sequence for sortase A comprises the amino acid sequence set forth in SEQ ID NO: 7 or SEQ ID NO: 8.

42. The PS binding agent of any one of embodiments 30 to 41, wherein the one or more isolated C-domains are covalently attached to the backbone by biotin.

43. The PS binding agent of any one of embodiments 30 to 42, wherein the one or more isolated C-domains are covalently attached to biotin by a sortase A or biotin ligase.

44. The PS binding agent of embodiment 43, wherein the biotin ligase comprises BirA.

45. The PS binding agent of any one of embodiments 30 to 44, wherein the one or more isolated C-domains are biotinylated.

46. The PS binding agent of embodiment 45, wherein the one or more isolated C-domains are biotinylated with sortase A or a biotin ligase.

47. The PS binding agent of embodiment 46, wherein the biotin ligase comprises BirA.

48. The PS binding agent of any one of embodiments 30 to 47, wherein the backbone comprises streptavidin, avidin or neutravidin.

49. The PS binding agent of any one of embodiments 30 to 48, wherein the backbone comprises streptavidin.

50. The PS binding agent of any one of embodiments 30 to 49, further comprising a label.

51. The PS binding agent of embodiment 50, wherein the label is selected from the group consisting of a tag, a fluorophore, a solid phase carrier, a microbubble, a linker, and an active site probe.

52. The PS binding agent of embodiment 51, wherein said solid phase carrier comprises a bead.

53. The PS binding agent of any one of embodiments 30 to 52, wherein the PS binding agent is capable of binding to PS on dead cells, dying cells, cell debris, activated thrombocytes and/or extracellular vesicles.

54. A composition comprising one or more PS binding agents of any one of embodiments 1 to 53.

55. A composition comprising at least one PS binding agent of any one of embodiments 1 to 53.

56. The composition of embodiment 54 or 55, further comprising a diagnostically acceptable excipient.

57. The composition of any one of embodiments 54 to 56, further comprising a calcium independent solution.

58. The composition of any one of embodiments 54 to 57, further comprising a calcium free solution.

59. A kit comprising the PS binding agent of any one of embodiments 1 to 53 or the composition of any one of embodiments 54 to 58.

60. A kit comprising one or more PS binding agents comprising one or more C-domains of a MFG-E8 protein and one or more additional molecules capable of linking said C-domains to each other.

61. The kit of embodiment 60, wherein the one or more additional molecules is streptavidin, avidin or neutravidin.

62. The kit of embodiment 60 or embodiment 61, wherein the one or more additional molecules further comprises a label.

63. The kit of embodiment 62, wherein said label is selected from the group consisting of a tag, a fluorophore, a solid phase carrier, a microbubble, a linker, and an active site probe.

64. The kit of embodiment 63, wherein said solid phase carrier is a bead.

65. The kit of any one of embodiments 59 to 64, wherein the one or more C-domains comprise 1, 2, 3, 4, 5, 6, 7, 8, 9 or 10 or more C-domains.

66. The kit of any one of embodiments 59 to 65, wherein the one or more C-domains comprise 10, 20, 30, 40, 50, 60, 70, 80, 90 or 100 or more C-domains.

67. The kit of any one of embodiments 59 to 66, wherein the one or more C-domains comprise 100 or more C-domains.

68. The kit of any one of embodiments 59 to 67, wherein the one or more PS binding agents comprise 1, 2, 3, 4, 5, 6, 7, 8, 9 or 10 or more PS binding agents.

69. The kit of any one of embodiments 59 to 68, wherein the one or more PS binding agents comprise 10, 20, 30, 40, 50, 60, 70, 80, 90 or 100 or more PS binding agents.

70. The kit of any one of embodiments 59 to 69, wherein the one or more PS binding agents comprise 100 or more PS binding agents.

71. A kit comprising at least two C-domains of a MFG-E8 protein and an additional molecule capable of linking said C-domains to each other.

72. The kit of any one of embodiments 59 to 71, wherein said C-domains comprise a C1-domain of MFG-E8 protein or fragment thereof and/or a C2-domain of MFG-E8 protein or a fragment thereof 73. The kit of any one of embodiments 59 to 72, wherein the C1-domain comprises an amino acid sequence having at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity with an amino acid sequence set forth in SEQ ID NO: 1.

74. The kit of any one of embodiments 59 to 73, wherein the C1-domain comprises the amino acid sequence set forth in SEQ ID NO: 1.

75. The kit of any one of embodiments 59 to 74, wherein the C2-domain comprises an amino acid sequence having at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or sequence identity with an amino acid sequence set forth in SEQ ID NO: 2.

76. The kit of any one of embodiments 59 to 75, wherein the C2-domain comprises the amino acid sequence set forth in SEQ ID NO: 2.

77. The kit of any one of embodiments 60 to 52, wherein said C-domains comprise a recognition sequence for said enzyme.

78. The kit of any one of embodiments 59 to 77, further comprising an enzyme for enzymatic modification of the C-domains.

79. The kit of embodiment 78, wherein the enzyme comprises a biotin ligase or a sortase A.

80. The kit of embodiment 79, wherein the biotin ligase comprises BirA. 81. The kit of any one of embodiments 59 to 80, further comprising biotin. 82. The kit of any one of embodiments 59 to 81, wherein said C-domains are biotinylated. 83. The kit of embodiment 82, wherein said C-domains are biotinylated with sortase A or a biotin ligase.

84. The kit of embodiment 83, wherein said biotin ligase is BirA.

85. The kit of any one of embodiments 59 to 84, further comprising a buffer that is substantially free of calcium.

86. Use of the PS binding agent of any one of embodiments 1 to 53, the composition of any one of embodiments 54 to 58, or the kit of any one of embodiments 59 to 85, for staining or detecting PS-positive dead or dying cells, cell debris, activated thrombocytes and/or extracellular vesicles, or for depleting or enriching PS-positive dead or dying cells, cell debris, activated thrombocytes and/or extracellular vesicles.

87. An in vitro method of staining PS-positive dead or dying cells, cell debris, activated thrombocytes and/or extracellular vesicles in a sample from a subject, the method comprising
a) contacting the sample with the PS binding agent of any one of embodiments 1 to 53 further comprising a fluorophore, and optionally
b) detecting said PS binding agent using an imaging method.

88. An in vitro method of staining PS-positive dead or dying cells, cell debris, activated thrombocytes and/or extracellular vesicles in a sample from a subject, the method comprising
a) contacting the sample with the PS binding agent of any one of embodiments 1 to 53 further comprising a fluorophore, under conditions to bind the PS binding agent to the sample,
b) detecting said PS binding agent using an imaging method.

89. An in vitro method of diagnosing a disease associated with the presence of PS positive dead or dying cells, cell debris, activated thrombocytes and/or extracellular, the method comprising
a) contacting a sample from a subject to be diagnosed with the PS binding agent of any one of embodiments 1 to 53 further comprising a fluorophore,
b) detecting said PS binding agent using an imaging method, thereby collecting imaging data, and
c) comparing the imaging data received in step b) to reference imaging data.

90. An in vitro method of diagnosing a disease associated with the presence of PS positive dead or dying cells, cell debris, activated thrombocytes and/or extracellular, the method comprising
a) contacting a sample from a subject to be diagnosed with the PS binding agent of any one of embodiments 1 to 53 further comprising a fluorophore, under conditions to bind the PS binding agent to the sample,
b) detecting said PS binding agent using an imaging method, thereby collecting imaging data, and
c) comparing the imaging data received in step b) to a reference imaging data.

91. The method of embodiment 88 or embodiment 90, wherein the conditions comprise a buffer that is substantially free of calcium.

92. The method of any one of embodiments 88 to 91, wherein an increased signal in the imaging data in the sample from the subject as compared to the reference imaging data indicates the presence of a disease associated with the presence of PS positive dead or dying cells, cell debris, activated thrombocytes and/or extracellular vesicles in said subject.

93. The method of any one of embodiments 88 to 92, wherein no difference in the signal in the imaging data in the sample from the subject as compared to the reference imaging data indicates the absence of a disease associated with the presence of PS positive dead or dying cells, cell debris, activated thrombocytes and/or extracellular vesicles in said subject.

94. An in vitro method of depleting or enriching PS-positive dead or dying cells, cell debris, activated thrombocytes and/or extracellular vesicles from a sample, comprising:
a) contacting said sample with the PS binding agent of any one of embodiments 1 to 53,
b) contacting said sample with a label capable of binding to the PS binding agent, and
c) depleting or enriching PS-positive dead or dying cells, cell debris and/or extracellular vesicles bound to said PS binding agent.

95. An in vitro method of depleting or enriching PS-positive dead or dying cells, cell debris, activated thrombocytes and/or extracellular vesicles from or in a sample, comprising:
a) contacting said sample with the PS binding agent of any one of embodiments 1 to 53 further comprising a label, and
b) depleting or enriching PS-positive dead or dying cells, cell debris and/or extracellular vesicles bound to said PS binding agent.

96. The method of embodiment 94 or embodiment 95, wherein the sample comprises living cells.

97. The method of any one of embodiments 94 to 96, wherein the label comprises a fluorophore, a solid phase carrier, or a microbubble.

98. The method of embodiment 97, wherein the solid phase carrier comprises a bead.

99. An in vitro method of depleting or enriching PS-positive cells from a sample, comprising:
a) contacting the sample with one or more PS binding agents of any one of embodiments 1 to 53, under conditions to bind the PS binding agent to the sample
b) contacting the sample with a label capable of binding to the PS binding agent, and
c) depleting or enriching PS-positive dead or dying cells, cell debris and/or extracellular vesicles bound to said PS binding agent.

100. An in vitro method of depleting or enriching PS-positive dead or dying cells, cell debris, activated thrombocytes and/or extracellular vesicles from or in a sample, comprising:
a) contacting said sample with one or more PS binding agents of any one of embodiments 1 to 53 further comprising a label, and
b) depleting or enriching PS-positive dead or dying cells, cell debris and/or extracellular vesicles bound to said PS binding agent.

101. The method of embodiment 99 or embodiment 100, wherein the sample comprises living cells.

102. The method of any one of embodiments 99 to 101, wherein the label comprises a fluorophore, a solid phase carrier, or a microbubble.

103. The method of embodiment 102, wherein the solid phase carrier comprises a bead.

104. The method of embodiment 103, wherein the bead comprises a magnetic bead.

105. The method of any one of embodiments 99 to 104, wherein the PS-positive cells comprise dead or dying cells, cell debris, activated thrombocytes and/or extracellular vesicles.

106. The method of any one of embodiments 102 to 105, wherein the conditions to bind the one or more PS bindings agent to the sample comprise an additional molecule selected from streptavidin, avidin or neutravidin.

107. The method of any one of embodiments 102 to 106, wherein the additional molecule links the one or more C-domains of the one or more PS binding agents.

108. The method of any one of embodiments 102 to 107, wherein depleting or enriching PS-positive cells comprises using a force selected from a magnetic field or buoyant/floatation properties 109. An in vitro method of depleting or enriching PS-positive cells from a sample, comprising:
a) contacting the sample with one or more PS binding agents of any one of embodiments 1 to 53, under conditions comprising a substantially calcium free buffer,
b) contacting the sample with an additional molecule selected from streptavidin, avidin or neutravidin, wherein the additional molecule links the one or more C-domains one or more PS binding agents.
c) contacting the sample with a label capable of binding to the PS binding agent, wherein the label comprises a fluorophore, a solid phase carrier, or a microbubble
d) depleting or enriching PS-positive cells using a force selected from a magnetic field or buoyant/floatation properties, wherein the PS-positive cells comprise dead or dying cells, cell debris and/or extracellular vesicles bound to said PS binding agent.

110. An in vitro method of depleting or enriching PS-positive cells from a sample, comprising:
a) contacting the sample with one or more PS binding agents of any one of embodiments 1 to 53, under conditions comprising a substantially calcium free buffer,
b) contacting the sample with an additional molecule selected from streptavidin, avidin or neutravidin, wherein the additional molecule links the one or more C-domains one or more PS binding agents,
c) contacting the sample with a label capable of binding to the PS binding agent, wherein the label comprises a solid phase carrier, and
d) depleting or enriching PS-positive cells using a magnetic field to separate the solid phase carrier bound PS binding agents, wherein the PS-positive cells comprise dead or dying cells, cell debris and/or extracellular vesicles bound to said PS binding agent.

111. An in vitro method of depleting or enriching PS-positive cells from a sample, comprising:
a) contacting one or more PS binding agents of any one of embodiments 1 to 53, with an additional molecule selected from streptavidin, avidin or neutravidin, wherein the additional molecule links the one or more C-domains one or more PS binding agents, under conditions comprising a substantially calcium free buffer,
b) contacting the one or more PS binding agents with a label capable of binding to the PS binding agent, wherein the label comprises a fluorophore, a solid phase carrier, or a microbubble,
c) contacting the one or more PS binding agents with the sample,
d) depleting or enriching PS-positive cells using a force selected from a magnetic field or buoyant/floatation properties, wherein the PS-positive cells comprise dead or dying cells, cell debris and/or extracellular vesicles bound to said PS binding agent.

112. An in vitro method of depleting or enriching PS-positive cells from a sample, comprising:
a) contacting one or more PS binding agents of any one of embodiments 1 to 53, with an additional molecule selected from streptavidin, avidin or neutravidin, wherein the additional molecule links the one or more C-domains one or more PS binding agents, under conditions comprising a substantially calcium free buffer,
b) contacting the one or more PS binding agents with a label capable of binding to the PS binding agent, wherein the label comprises a solid phase carrier
c) contacting the one or more PS binding agents with the sample,
d) depleting or enriching PS-positive cells using a magnetic field to separate the solid phase carrier bound PS binding agents, wherein the PS-positive cells comprise dead or dying cells, cell debris and/or extracellular vesicles bound to said PS binding agent.

113. The method of any one of embodiments 108 to 112, wherein the solid phase carrier comprises a magnetic bead.

114. A method of producing a PS binding agent comprising at least two C-domains of MFG-E8 protein, the method comprising
a) contacting at least two C-domains of a MFG-E8 protein with an additional molecule capable of linking said C-domains to each other under conditions allowing the formation of a PS binding agent comprising at least two C-domains of MFG-E8 protein, and optionally
b) recovering the produced PS binding agent.

115. The method of embodiment 114, wherein theC-domain is any one of a C1-domain of MFG-E8 protein, a C2-domain of MFG-E8 protein or a fragment thereof.

116. The method of embodiment 115, wherein the C1-domain comprises an amino acid sequence having at least about 60% sequence identity with an amino acid sequence of SEQ ID NO: 1.

117. The method of embodiment 115 or 116, wherein the C2-domain comprises an amino acid sequence having at least about 60% sequence identity with an amino acid sequence of SEQ ID NO: 2.

118. The method of any one of embodiments 114 to 117, wherein the C1-domain comprises the amino acid sequence of SEQ ID NO: 1.

119. The method of any one of embodiments 114 to 118, wherein the C2-domain comprises the amino acid sequence of SEQ ID NO: 2.

120. The method of any one of embodiments 114 to 119, wherein said C-domains are biotinylated.

121. The method of embodiment 120, wherein the C-domains are biotinylated with sortase A or a biotin ligase.

122. The method of embodiment 121, wherein the biotin ligase is BirA.

123. The method of any one of embodiments 114 to 122, wherein said C-domains are directly or indirectly linked to another.

124. The method of embodiment 123, wherein said C-domains are covalently linked.

125. The method of any one of embodiments 114 to 124, further comprising labelling said PS binding agent.

126. The method of any one of embodiments 114 to 125, wherein said additional molecule is streptavidin, avidin or neutravidin.

127. The method of any one of embodiments 114 to 126, wherein said additional molecule further comprises a label.

128. The method of embodiment 127, wherein said label is selected from the group consisting of a tag, a fluorophore, a solid phase carrier, a microbubble, a linker, and an active site probe.

129. The method of embodiment 128, wherein said solid phase carrier is a bead.

130. An in vitro method of detecting PS-positive dead or dying cells, cell debris and/or extracellular vesicles, preferably PS-positive cell-associated extracellular vesicles in an animal, preferably a mouse, to whom a PS binding agent of any one of embodiments 1 to 53 further comprising a fluorophore has been pre-delivered, comprising:
a) analyzing a sample obtained from said animal,
b) detecting said pre-delivered PS binding agent using an imaging method, thereby collecting imaging data, and
c) comparing the imaging data received in step b) to reference imaging data.

131. The method of embodiment 130, wherein an increased signal in the imaging data from said animal as compared to reference imaging data indicates the presence of PS-positive dead or dying cells, cell debris, activated thrombocytes and/or extracellular vesicles in said animal.

132. The method of embodiment 131, wherein no difference in the imaging signal in the imaging data from said animal as compared to reference imaging data indicates no presence of PS-positive dead or dying cells, cell debris, activated thrombocytes and/or extracellular vesicles in said animal.

The above examples are provided to illustrate the disclosure but not to limit its scope. Other variants of the disclosure will be readily apparent to one of ordinary skill in the art and are encompassed by the appended claims. All publications, databases, internet sources, patents, patent applications, and accession numbers cited herein are hereby incorporated by reference in their entireties for all purposes.

What is claimed is:

1. A phosphatidylserine (PS) binding agent comprising one or more isolated C-domains of a milk fat globule-EGF factor 8 (MFG-E8) protein.

2. The PS binding agent of claim 1, wherein the PS binding agent comprises two, three, four, or more isolated C-domains of the MFG-E8 protein.

3. The PS binding agent of claim 1, wherein the C-domains comprise a C1-domain of the MFG-E8 protein or a functional fragment thereof, and a C2-domain of the MFG-E8 protein or a functional fragment thereof.

4. The PS binding agent of claim 1, wherein the C-domains comprise a C1-domain of the MFG-E8 protein or a functional fragment thereof, or a C2-domain of the MFG-E8 protein or a functional fragment thereof.

5. The PS binding agent of claim 3, wherein the C1-domain comprises an amino acid sequence having at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of SEQ ID NO: 1.

6. The PS binding agent of claim 3, wherein the C1-domain comprises the amino acid of SEQ ID NO: 1.

7. The PS binding agent of claim of 3, wherein the C2-domain comprises an amino acid sequence having at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of SEQ ID NO: 2.

8. The PS binding agent of claim 3, wherein the C2-domain comprises the amino acid sequence of SEQ ID NO: 2.

9. The PS binding agent of claim 1, wherein the PS binding agent comprises two or more C-domains that are covalently linked.

10. The PS binding agent of claim 1, further comprising a recognition sequence for enzymatic modification.

11. The PS binding agent of claim 10, wherein the recognition sequence is for a biotin ligase or for a sortase A.

12. The PS binding agent of claim 10, wherein the recognition sequence for the sortase A comprises the amino acid sequence of SEQ ID NO: 7 or SEQ ID NO: 8.

13. The PS binding agent of claim 1, wherein the one or more C-domains are covalently attached to a biotin.

14. The PS binding agent of claim 13, wherein the one or more C-domains are covalently attached to the biotin by a sortase A or a biotin ligase.

15. The PS binding agent of claim 1, wherein the PS binding agent comprises two or more C-domains that are linked directly to each other.

16. The PS binding agent of claim 1, wherein the PS binding agent comprises two or more C-domains that are linked to each other via an additional molecule.

17. The PS binding agent of claim 16, wherein the additional molecule comprises streptavidin, avidin, or neutravidin.

18. The PS binding agent of claim 1, further comprising a label.

19. A kit comprising one or more PS agents comprising two or more C-domains of a milk fat globule-EGF factor 8 (MFG-E8) protein and one or more additional molecules capable of linking the C-domains to each other.

20. An in vitro method of staining PS-positive dead or dying cells, cell debris, activated thrombocytes, and/or extracellular vesicles in a sample from a subject, the method comprising:
a) contacting the sample with the PS binding agent of claim 1 and a fluorophore, under conditions to bind the PS binding agent to the sample; and
b) detecting said PS binding agent using an imaging method.
